(19) United States
(12) Patent Application Publication
(10) Pub. No.: US 2026/0273767 A1
(43) Pub. Date: Sep. 17, 2026

(54) SYSTEM

(71) Applicant: SoftBank Group Corp., Tokyo (JP)

(72) Inventor: Fumi NAKAMOTO, Tokyo (JP)

(73) Assignee: SoftBank Group Corp., Tokyo (JP)

(21) Appl. No.: 19/547,710

(22) Filed: Feb. 24, 2026

Related U.S. Application Data
(60) Provisional application No. 63/772,982, filed on Mar. 17, 2025.

(51) Int. Cl. B25J 11/00; B25J 9/16; G06Q 10/083; G06V 10/82; G06V 40/16; G10L 15/16; G10L 25/63; G16H 20/60

(52) U.S. Cl. CPC B25J 11/0045; B25J 9/1674; G06Q 10/0843; G06V 10/82; G06V 40/174; G10L 15/16; G10L 25/63; G16H 20/60

(57) ABSTRACT

A system includes circuitry configured to receive, via a communication interface coupled to a packet-switched network, attribute data records associated with a plurality of user profiles, each record including user-specific parameters. The circuitry computes, based on the attribute data records and reference data stored in memory, per-user requirement values for the user profiles. The circuitry generates a composite output plan by inputting the per-user requirement values into a data generation model, the composite output plan satisfying constraint conditions derived from the per-user requirement values. Based on the composite output plan, the circuitry generates instruction data including a sequence of operational parameters and transmits the instruction data to a controlled apparatus over the packet-switched network to cause execution of operations in accordance with the operational parameters.

### CROSS-REFERENCE TO RELATED APPLICATION

This application is based on and claims priority under 35 USC 119 (e) from U.S. Provisional Application No. 63/772,982 filed on Mar. 17, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional meal planning and cooking support systems require a user to manually perform multiple complex tasks, such as collecting and managing detailed health information of each family member, calculating necessary nutrients according to individual health states and nutritional needs, constructing a nutritionally balanced menu that can be enjoyed by the entire family, creating a corresponding ingredient list, placing and scheduling online orders for the ingredients, generating appropriate recipes for available cooking devices, and actually performing and supervising the cooking process. As a result, a user, for example a working parent, faces a significant time burden and cognitive load when attempting to maintain a healthy and customized diet for all family members, including children and elderly persons having specific health conditions, allergies, or dietary restrictions. In addition, conventional systems often do not integrate dynamic order and delivery coordination with menu planning, and do not provide automatic, robot-optimized cooking control based on the planned menu and calculated nutrition. Furthermore, there is insufficient capability to automatically generate cooking procedures adapted to the functions and constraints of a particular cooking robot and to monitor and adjust the robot's operation in real time. Therefore, there is a need for a system that centrally manages health status data of each family member, automatically calculates required nutrients, generates a family-wide optimized menu, coordinates ingredient procurement, generates robot-optimized recipes, and controls a cooking robot so that a nutritionally balanced meal can be prepared with reduced burden on the user.

### SUMMARY

In order to solve the above-described problems, the present invention provides a system comprising a processor configured to execute a series of integrated functions as recited in the claims. Specifically, the processor is configured to receive, via a health status data input interface, health status data for each member of a family including at least age, gender, health condition, allergy information, food preferences, and special nutritional needs, and to calculate, by a nutrition balance calculation function, required nutrients for each family member based on the received health status data. The processor is further configured to generate, by a menu generation function, a nutritionally balanced menu that is enjoyable for the entire family based on an output of the nutrition balance calculation function, and to create, by an ingredient list creation function, an ingredient list of ingredients required for the generated menu. The processor is configured to transmit, by an order and delivery coordination function, the ingredient list to an affiliated online ordering and delivery service to automatically procure the ingredients, and to automatically adjust a delivery schedule such that the ingredients are delivered at a date and time designated by a user. In addition, the processor is configured to generate, by a recipe generation function, cooking procedures based on the generated menu and optimized for functions of a cooking robot, to output, by a cooking instruction function, detailed instructions to the cooking robot based on the generated cooking procedures so that the cooking robot can cook efficiently, and to control, by a cooking robot control function, an operation of the cooking robot so that the cooking robot automatically cooks in accordance with the instructions while the operation is monitored in real time and adjusted as needed. By these means, the system enables automatic and integrated execution of health data handling, nutrition calculation, menu generation, ingredient procurement, recipe generation, and cooking robot control, thereby significantly reducing the burden on the user while providing meals tailored to the health conditions and preferences of all family members.

The term “processor” refers to one or more hardware processing units, such as a central processing unit (CPU), a graphics processing unit (GPU), a microcontroller, or a combination thereof, capable of executing instructions and implementing the functions recited in the claims.

The term “health status data” refers to information related to physical or medical conditions of a person, including at least age, gender, current or past health conditions, allergy information, food preferences, and special nutritional needs.

The term “health status data input interface” refers to a hardware and/or software interface, such as a graphical user interface, web interface, or application interface, through which health status data for one or more family members is received.

The term “nutrition balance calculation function” refers to a function implemented by the processor that processes received health status data and calculates required nutrients for each person, including at least caloric requirements and amounts of macronutrients and/or micronutrients.

The term “required nutrients” refers to target amounts or ranges of nutrients, including at least calories and at least one of proteins, fats, carbohydrates, vitamins, or minerals, that are determined to be appropriate for an individual based on health status data.

The term “menu generation function” refers to a function implemented by the processor that generates one or more proposed meals or menus based on outputs of the nutrition balance calculation function, such that the generated menu is nutritionally balanced and suitable for consumption by a family.

The term “nutritionally balanced menu” refers to a set of one or more dishes whose combined nutrients are configured to satisfy, within predetermined tolerances, the required nutrients calculated for the relevant persons or family members.

The term “ingredient list creation function” refers to a function implemented by the processor that derives and outputs a list of ingredients and corresponding quantities required to prepare the dishes included in a generated menu.

The term “ingredient list” refers to structured data identifying one or more ingredients and associated quantities and optionally units, which collectively represent the materials needed to prepare the generated menu.

The term “order and delivery coordination function” refers to a function implemented by the processor that generates and transmits ordering information based on an ingredient list to an external online ordering and delivery service, and coordinates or adjusts a delivery schedule for the ordered ingredients.

The term “online ordering and delivery service” refers to an external system or service, typically provided by a retailer or logistics provider, that receives electronic orders for goods and arranges delivery of those goods to a specified address.

The term “automatic procurement” refers to the process in which the system, without manual entry of individual item orders by the user, prepares and transmits order information based on the ingredient list to the online ordering and delivery service and receives confirmation of the order.

The term “delivery schedule” refers to information specifying at least a date and optionally a time or time window at which ordered ingredients are to be delivered to a designated location.

The term “recipe generation function” refers to a function implemented by the processor that generates cooking procedures, including steps, conditions, and parameters, based on a generated menu and optionally tailored to capabilities of a specific cooking robot.

The term “cooking procedures” refers to ordered instructions for preparing one or more dishes, including at least a sequence of steps, and optionally associated times, temperatures, actions, or equipment settings.

The term “cooking instruction function” refers to a function implemented by the processor that outputs, transmits, or otherwise provides instructions, based on generated cooking procedures, to a cooking robot so that the cooking robot can perform at least part of the cooking process.

The term “cooking robot control function” refers to a function implemented by the processor that controls operation of a cooking robot, including starting, stopping, adjusting, and monitoring cooking actions, so that the cooking robot automatically cooks according to provided instructions.

The term “cooking robot” refers to a cooking apparatus, equipped with actuators and controllers, that can automatically execute cooking-related operations such as heating, stirring, mixing, cutting, or dispensing under control of the system.

The term “family” refers to a plurality of persons sharing living arrangements or meals, including but not limited to children, adults, and elderly persons, for whom meals are to be jointly planned or prepared.

The term “user” refers to a person who operates the system or terminal, enters or confirms data, and receives outputs, and who may be a member of the family or a caregiver.

The term “user interface” refers to hardware and/or software components, such as display screens, input devices, and graphical user interface elements, that enable interaction between the user and the system, including input of health status data and display of menus, ingredient lists, and cooking procedures.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

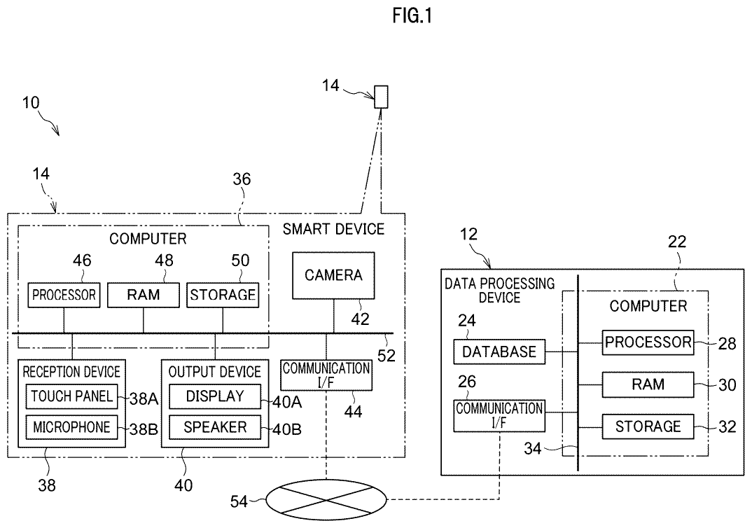

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

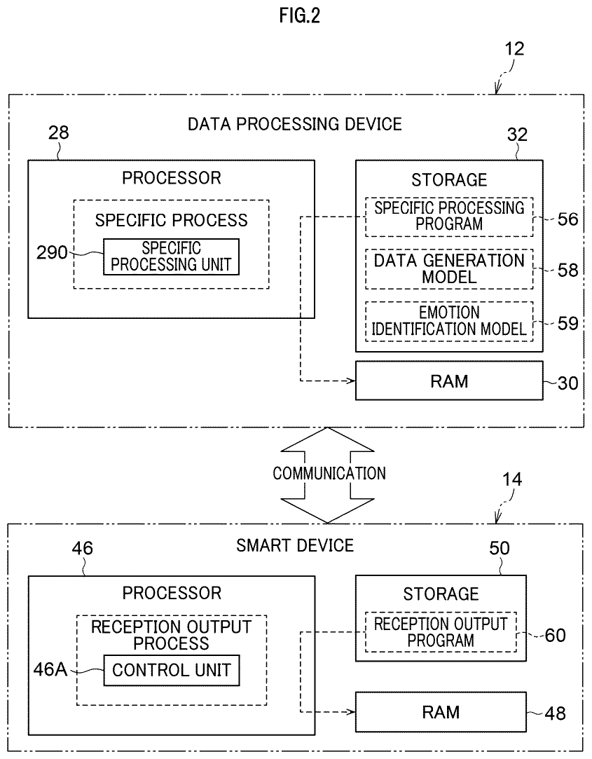

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented meal planning and cooking-assistance systems typically execute a set of isolated application functions on a server, such as storing user profiles, running fixed nutrition calculators, or providing static recipe databases. In such systems, when a user's household composition, health status, or device capability changes, the system generally relies on preprogrammed rule sets and manually curated content. As a result, the system often fails to adapt to complex, multi-constraint scenarios, such as simultaneously satisfying individual nutritional requirements, allergy constraints, delivery constraints, and cooking-device constraints. Furthermore, conventional systems treat interaction with machine-learning components, where present, as a one-way text generation step, without maintaining structured associations between prompts, model responses, and underlying user data in a way that can be re-used to refine subsequent computations or control flows.

From the viewpoint of computer technology, these conventional approaches exhibit several technical problems. First, nutrition planning logic and cooking-device control logic are separated from the generative processing performed by machine-learning models, so that the computing resources of the server cannot effectively coordinate heterogeneous data processing pipelines (nutrition computation, menu generation, shopping optimization, and device control). This separation results in redundant computations and frequent failure cases in which the server must repeatedly regenerate menus or recipes due to constraint violations, thereby increasing processor load and network traffic. Second, conventional systems do not integrate generative AI model outputs into closed-loop feedback control for cooking devices. They typically convert static recipes into commands once, and do not adapt the control commands based on real-time sensor feedback in a structured, automated way, which leads to unreliable cooking results and inefficient use of processing and communication resources. Third, existing server architectures do not maintain prompt sentences and generative AI responses as structured learning data linked to household composition and health-related information, so the server cannot programmatically improve the generation of subsequent prompts or the selection of constraints, and must repeatedly perform similar validation and correction operations. Accordingly, there is a need for improved computer-implemented techniques that tightly integrate generative AI model processing with deterministic numerical computations, external commerce and logistics interfaces, and real-time control of cooking apparatuses. There is a further need for a server architecture and processing method that: (i) generates and refines prompt sentences based on structured household and health-related information; (ii) performs automatic validation and regeneration of menus and recipes by combining generative AI outputs with constraint-checking logic; (iii) generates structured control-command sequences from AI-generated recipes; and (iv) dynamically updates such sequences based on sensor feedback from a cooking apparatus. Addressing these needs would improve the operation of the server as a technical system, by reducing unnecessary regeneration cycles, lowering the amount of data transmitted over networks, and increasing the reliability and efficiency of automated cooking control.

The specific processing by the specific processing unit 290 of the data processing device 12 in Example 1 is realized by the following means.

The present invention provides a server comprising a processor configured to acquire household composition information and health-related information for each member of a household via an input interface, store the household composition information and the health-related information in a storage device, and perform numerical computation based on the household composition information, the health-related information, and nutrition reference information stored in the storage device, to calculate nutrition requirement information for each member; a prompt-generation and menu-generation component configured to generate structured information including the nutrition requirement information, the household composition information, and the health-related information, convert at least part of the structured information into a character string as a prompt sentence, transmit the prompt sentence to a generative AI model, receive response data from the generative AI model, and generate menu information for a predetermined period based on the response data; a shopping-list generation component configured to extract ingredient information for a plurality of dishes included in the menu information, perform aggregation processing and unit conversion processing for each ingredient to calculate a required ingredient amount, acquire ingredient candidate information corresponding to the required ingredient amount from an external product information providing device, and generate ingredient list information; an order-scheduling component configured to acquire, based on the ingredient list information and delivery-preference time-band information of a user stored in the storage device, delivery-slot information from an external goods-delivery management device, automatically determine a delivery schedule consistent with the delivery-preference time-band information, and transmit order information to the goods-delivery management device; a recipe-generation component configured to generate cooking procedure data indicating cooking steps for each dish based on the menu information and the nutrition requirement information, and generate structured data including operation parameters for a cooking apparatus from the cooking procedure data by using, in at least part, a prompt sentence and a response of the generative AI model; a control-command generation and transmission component configured to generate a sequence of control commands including temperature, time, stirring speed, and heating state based on the structured data, and transmit the sequence of control commands to the cooking apparatus; a feedback-control component configured to receive temperature information, time information, and operation-state information from the cooking apparatus, compare a target value in the sequence of control commands with the temperature information, the time information, and the operation-state information to determine a cooking state, and generate, according to a determination result, additional control commands including extension of cooking time or change of temperature and transmit the additional control commands to the cooking apparatus; and a learning-data management component configured to store, in association, at least one of the prompt sentence and the response data of the generative AI model, and the household composition information and the health-related information, in the storage device as learning data, and update a generation condition of a subsequent prompt sentence based on the learning data. This enables the server to technically improve coordination between deterministic numerical processing and generative AI processing, to automatically regenerate menus and recipes only when constraint violations are detected, to generate and adapt structured control-command sequences for a cooking apparatus based on real-time sensor feedback, and to refine prompt-sentence generation over time using stored associations between prompts, model responses, and user data, thereby enhancing overall computational efficiency, reducing unnecessary network and processing overhead, and increasing reliability and accuracy of automated meal planning and cooking control.

The term “household composition information” refers to information indicating a configuration of a household, including at least a number of members, an age of each member, a sex of each member, and optionally additional identification or role information for each member. The term “health-related information” refers to information associated with a physiological or medical state of a household member, including at least disease information, allergy information, intake-restriction information, and optionally lifestyle information and dietary preference information.

The term “nutrition reference information” refers to information stored in a storage device that defines recommended intake values of nutrients, including at least energy, macronutrients, and micronutrients, in association with attributes such as age, sex, and health condition.

The term “nutrition requirement information” refers to information indicating a calculated amount of nutrients required for each household member, the information being obtained by numerical computation based on the household composition information, the health-related information, and the nutrition reference information.

The term “structured information” refers to information organized according to a predetermined data structure, such as a record, table, or hierarchical format, in which individual fields, including the nutrition requirement information, the household composition information, and the health-related information, are explicitly represented and machine-readable.

The term “prompt sentence” refers to a character-string representation of at least part of the structured information, formatted according to a predetermined style, and transmitted to a generative AI model as an input instruction for causing the generative AI model to generate response data.

The term “generative AI model” refers to a machine-learned model capable of generating data, including character-string data and structured data, in response to an input such as a prompt sentence, the machine-learned model being trained using a dataset and implemented on one or more computing devices.

The term “response data” refers to data output from the generative AI model in response to a prompt sentence, the data including at least one of natural-language text and structured data representing menu information, ingredient information, or recipe information.

The term “menu information” refers to information indicating one or more dishes planned for consumption during a predetermined period, including at least identifiers of the dishes and, optionally, associated ingredient information and scheduling information.

The term “ingredient information” refers to information indicating a type and an amount of a material used for preparing a dish, the information including at least an ingredient name, a quantity, and a unit. The term “required ingredient amount” refers to an amount of an ingredient that is calculated by aggregating ingredient information across a plurality of dishes included in the menu information and by performing unit conversion processing.

The term “ingredient candidate information” refers to information obtained from an external product information providing device that indicates one or more purchasable items corresponding to a required ingredient amount, including at least a product identifier, a package size, and optionally price or availability information.

The term “ingredient list information” refers to information representing a list of ingredients or corresponding purchasable items required for preparing dishes included in the menu information, the information including at least an association between each required ingredient amount and corresponding ingredient candidate information.

The term “delivery-preference time-band information” refers to information indicating one or more time ranges preferred by a user for receiving delivery of goods, including at least a day or date and a time interval.

The term “delivery-slot information” refers to information obtained from an external goods-delivery management device that indicates available delivery time periods for delivering goods to a destination.

The term “delivery schedule” refers to information indicating a selected delivery time period and associated order information for goods to be delivered, the schedule being determined based on the delivery-preference time-band information and the delivery-slot information.

The term “order information” refers to information transmitted to a goods-delivery management device to request delivery of goods, including at least product identifiers, quantities, a delivery address, and a delivery schedule.

The term “cooking procedure data” refers to information indicating an ordered sequence of cooking steps for preparing a dish, including at least a description of each step and one or more associated operation parameters.

The term “structured data including operation parameters for a cooking apparatus” refers to data derived from the cooking procedure data and formatted according to a predetermined schema such that operation parameters, including at least temperature, time, and stirring speed, are explicitly represented for control of a cooking apparatus.

The term “sequence of control commands” refers to an ordered set of machine-readable instructions that specify operational states of a cooking apparatus, including at least commands for setting temperature, setting time, setting stirring speed, and controlling heating.

The term “cooking apparatus” refers to an apparatus configured to perform food preparation operations, the apparatus including at least one heating element, at least one motion element, and one or more sensors, and being controllable by receiving the sequence of control commands from a server.

The term “temperature information” refers to information indicating a measured temperature value acquired from at least one temperature sensor of the cooking apparatus.

The term “time information” refers to information indicating an elapsed time or a current time associated with an execution of at least one control command in the sequence of control commands. The term “operation-state information” refers to information indicating an operational state of the cooking apparatus, including at least an execution status of control commands and optionally a stirring-speed state, a heating state, and an error state.

The term “cooking state” refers to a state representing a progress or condition of a cooking process, determined by comparing a target value defined in the sequence of control commands with the temperature information, the time information, and the operation-state information.

The term “additional control commands” refers to one or more control commands generated after initial generation of the sequence of control commands, the additional control commands including at least a command for extending a cooking time or changing a temperature based on a determination result regarding the cooking state.

The term “learning data” refers to information stored in a storage device that associates at least one of prompt sentences and response data of the generative AI model with the household composition information and the health-related information, the information being used to update a generation condition of subsequent prompt sentences.

The term “generation condition of a subsequent prompt sentence” refers to one or more parameters or rules used by the processor for constructing a future prompt sentence, the parameters or rules being updated based on the learning data so as to modify content or structure of the future prompt sentence.

In one embodiment, a server implements the claimed system as a network-connected computing device comprising at least one central processing unit, a main memory, a non-volatile storage device, a network interface, and an interface to at least one cooking apparatus. The server executes an operating system such as a general-purpose server operating system and runs application software implemented, for example, in a programming language with numerical computation libraries. The server stores program modules corresponding to the input interface, nutrition computation, prompt-generation, menu-generation, shopping-list generation, order scheduling, recipe generation, control-command generation, feedback-control, and learning-data management. A terminal implements a client device such as a smartphone, a tablet computer, or a personal computer, executing a client application or a web browser. The terminal includes a display unit, an input unit, a network interface, and a local memory. The terminal executes a graphical user interface that allows a user to input household composition information and health-related information, and to review menu information, ingredient list information, and cooking status. The user operates the terminal by touching a touch panel, typing on a keyboard, or using other input devices. A user uses the terminal to provide household composition information and health-related information. The terminal converts user inputs into structured data objects, for example records including fields such as member identifier, age, sex, disease code, allergy code, and intake restriction flags. The terminal transmits such structured data objects to the server over a network as formatted messages.

The server receives the structured data objects and stores them in a database management system such as a relational database. The server maintains tables for household members, health profiles, nutrition requirement profiles, menu entries, ingredient lists, and control sequences. The server indexes these tables by user identifier and timestamps so that subsequent retrieval and correlation operations can be performed by efficient key-based queries.

The server executes a nutrition computation module that performs numerical operations on the stored household composition information and health-related information by referencing nutrition reference information stored in tables. The server stores the nutrition reference information as records associating demographic attributes and health conditions with recommended intake values for energy, macronutrients, and micronutrients. The server multiplies per-kilogram or per-day base values by body mass or age factors and adjusts values by condition-specific modifiers. By implementing these calculations as vectorized numeric operations in the main memory, the server reduces repeated computations and achieves higher throughput relative to per-request calculations using conventional rule engines.

The server implements a generative AI model as a neural-network-based language model. In one embodiment, the generative AI model is a transformer-based neural network including multiple layers of self-attention and feed-forward sublayers. The generative AI model is trained using supervised learning and optionally fine-tuned with instruction-following data. During training, the model parameters are updated by back-propagation with a loss function such as cross-entropy between predicted tokens and target tokens. The server, or another computing system, performs the training by iteratively applying gradient descent or a variant such as Adam optimization, updating weight matrices and bias vectors in attention and feed-forward layers. The model learns to map sequences of tokens to probability distributions over next tokens.

The server uses the generative AI model at inference time by encoding prompt sentences into token sequences, providing the token sequences as input to the transformer model, and obtaining output token distributions from the final layer. The server generates output text by sampling or selecting tokens based on a decoding strategy such as greedy decoding or top-k sampling. The server executes this inference on an accelerator such as a graphics processing unit or a specialized matrix processor to reduce inference time.

The server converts structured information about nutrition requirement information, household composition information, and health-related information into prompt sentences. The server applies deterministic templates that arrange such information into natural-language text, thus creating prompts that encode multiple constraints. For example, the server may generate a prompt sentence of the form:

“Generate a 7-day dinner menu for a family. One member is a child with a nut allergy and a preference for mildly spicy food. Another member is an elderly person with type 2 diabetes who requires low-sugar and low-salt meals. Use seasonal winter ingredients such as root vegetables. Ensure each day's menu meets recommended nutritional guidelines, avoids nuts entirely, limits sugar and salt for the elderly member, and provides sufficient calcium and vitamin D for the child. Return the result as a list of days with dishes and ingredients.”

The server transmits such a prompt sentence to the generative AI model and receives response data. The server parses the response data into structured menu information. The server then validates the menu information against hard constraints stored in the database. The server compares ingredient names against allergy information and intake-restriction information and rejects or modifies entries that violate constraints. When violations are detected, the server generates additional prompt sentences that explicitly reference the violations, such as:

“Regenerate the menu for day 3. The previous menu included nuts. Please strictly avoid any nut ingredients while maintaining the same nutritional targets.”

By implementing this validation and regeneration loop at the server, the system reverses the conventional pattern where a human checks generated menus. The server programmatically enforces rules by combining deterministic constraint checking with generative pattern completion. This combination improves the technical performance of menu generation because the server avoids presenting invalid menus to the terminal and reduces the number of manual corrections. The server generates ingredient list information by aggregating ingredient information across all dishes in the menu information. The server maintains ingredient records with normalized units and performs summation and unit conversion operations using numeric routines. The server retrieves product information from an external product information providing device via an application programming interface. The server maps generic ingredients to specific product identifiers by matching ingredient attributes to product metadata, and then selects combinations of products that satisfy required ingredient amounts.

The server generates order information and delivery schedules. The server processes delivery-preference time-band information and delivery-slot information using interval comparison algorithms. The server searches for time slots that overlap preferred time bands and that can accommodate all required deliveries. By automating this matching and optimizing the selection of delivery windows, the server reduces the number of API calls to external goods-delivery management devices, which decreases network load and improves response latency relative to naïve sequential scheduling.

The server implements a recipe generation module that uses both deterministic rule-based transformation and generative AI model output. The server generates prompt sentences including device-capability information and dish definitions. For example, the server may generate a prompt sentence as follows:

“Generate a detailed, step-by-step recipe for ‘Chicken and root vegetable soup’ optimized for a multifunction cooking apparatus that supports heating, stirring at variable speeds, and timed operations. Use metric units. Specify exact times, temperatures, stirring speeds, and alerts for adding ingredients. The recipe must avoid dairy and nuts and must remain low in salt.”

The server transmits this prompt sentence to the generative AI model and receives structured text describing steps, including timing and temperature. The server parses the text into structured data representing operation parameters such as target temperature in degrees, stirring speed levels, and step durations. The server ensures that the parsed parameters fall within safe ranges for the cooking apparatus specified in device-capability information.

The server generates a sequence of control commands from the structured operation parameters. The server encodes commands as records specifying operation type, parameter values, and execution order. The server transmits the sequence of control commands to the cooking apparatus using a communication protocol such as a message-oriented protocol over a wireless or wired network. The server expects acknowledgments and status messages from the cooking apparatus and maintains a state machine representing the current command index and apparatus state.

The cooking apparatus includes at least one sensor for temperature and may include additional sensors such as current sensors, lid position sensors, or weight sensors. The cooking apparatus measures sensor values and transmits them to the server. The server receives temperature information, time information, and operation-state information and compares these values with target values in the control-command sequence. The server determines a cooking state by computing differences between measured and target values and by applying threshold criteria, such as whether temperature has stabilized within a tolerance range or whether expected time has elapsed. The server generates additional control commands when deviations are detected. For example, if temperature remains below a target threshold for longer than a timeout duration, the server increases heating duration or adjusts heating power by generating commands that extend cooking time or adjust temperature setpoints. The server thereby implements a closed-loop feedback control scheme at the application level, rather than simply executing a static script. This feedback control reduces undercooking or overcooking and enables the server to compensate for variations in ingredient mass or ambient conditions.

The server stores prompt sentences, generative AI responses, associated household composition information, and health-related information as learning data. The server maintains a data structure that links prompt identifiers, model outputs, validation results, and user feedback such as ratings. The server periodically analyzes this learning data using statistical methods to adjust prompt-generation parameters. For example, the server may increase the emphasis on certain constraint phrases in prompts when past outputs have shown frequent violation of those constraints, or may modify temperature or time ranges included in recipe prompts when user feedback indicates systematic overcooking.

The server improves computational efficiency through this learning-based adaptation. Because the server refines prompts based on previous errors and validation failures, the generative AI model is more likely to produce valid menus and recipes in fewer iterations. This reduces the number of calls to the generative AI model, decreases central processing unit and accelerator usage, and reduces network payloads. The server further optimizes database access by caching frequently used nutrition reference information and computed nutrition requirement information in memory, thereby reducing disk access operations.

The system provides technical improvements over conventional implementations in several respects. First, the server integrates deterministic numerical computation, structured prompt generation, generative output validation, and feedback control of physical apparatuses in a single coordinated architecture. This integration reduces redundant recalculation and re-transmission of data and improves throughput when serving multiple households. Second, by transforming generative AI model outputs into structured control commands for a cooking apparatus and by updating such commands based on sensor feedback, the server extends generative processing beyond abstract text generation to real-time control of a physical machine. This results in more precise cooking temperature control and more consistent dish outcomes than manual interpretation of text recipes. Third, the server manages learning data in a way that directly affects computational behavior. Rather than relying on static templates, the server dynamically modifies prompt construction rules based on past constraint violations, thereby increasing the precision of prompts with respect to system constraints. This systematic management of prompts and responses constitutes an improvement in data management and model-interaction techniques, yielding lower error rates and reduced need for human intervention.

In alternative embodiments, the server may use different model architectures, such as recurrent neural networks or convolutional sequence models, provided that these models are capable of generating text from prompt sentences. The server may also adjust the granularity of structured data, for example by representing recipes at a higher-level abstraction of phases or at a lower level of individual actuator commands. The terminal may be implemented as a dedicated appliance or as software integrated into a household hub device. The cooking apparatus may vary in capabilities, and the server can adapt its command generation by referencing device-capability profiles.

The user interacts with the system primarily through the terminal but does not directly manage low-level control of the cooking apparatus. Instead, the server performs non-conventional processing by combining nutrition mathematics, generative text modeling, constraint validation, and real-time feedback control. This combination goes beyond simple automation of human planning because the server employs internal data structures and control logic that are specifically designed for efficient parallel processing, constraint propagation, and feedback-driven command adjustment. As a result, the system achieves improved processing speed, improved accuracy of menu and recipe generation under complex constraints, reduced error rates in cooking outcomes, and reduced overall communication load between server, external services, and cooking apparatuses.

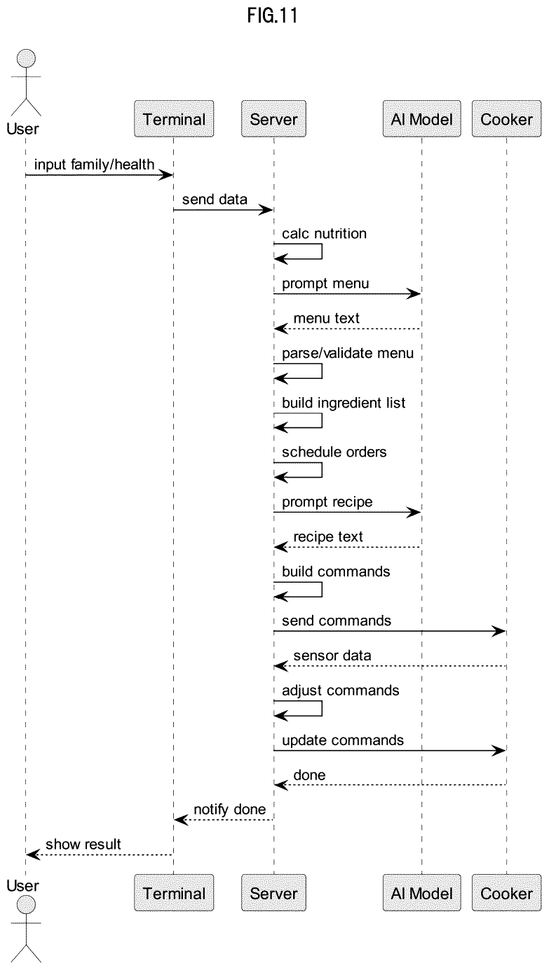

The following describes the processing flow using FIG. 11.

### Step 1

User operates the terminal to start an application or open a web page for family meal management.

Terminal displays input screens for household composition information and health-related information, including fields for age, sex, disease, allergy, intake restriction, and preference.

Input: Raw user actions (touches, keystrokes) and initial empty form fields.

Processing: Terminal converts user actions into structured records, validates formats (e.g., numeric age, required fields), and creates an internal data object with key-value pairs for each household member.

Output: Structured household data object ready to be transmitted to the server.

### Step 2

Terminal sends the structured household data object to the server over a network using a request message.

Server receives the request message and parses the household data object from the message body.

Input: Structured household data object in the request message.

Processing: Server validates the data (e.g., allowed ranges for age, supported disease and allergy codes), normalizes codes (e.g., maps text labels to internal identifiers), and checks for missing or inconsistent entries.

Output: Normalized and validated household composition information and health-related information stored temporarily in server memory.

### Step 3

Server stores the normalized household composition information and health-related information into a database.

Input: Normalized household data in memory.

Processing: Server executes database insert and update operations, creating or updating records in tables for members, health profiles, and preferences, and generates indexes or keys for later retrieval.

Output: Persistent records representing household composition information and health-related information, stored with identifiers in the database.

### Step 4

Server retrieves nutrition reference information from the database or a cached memory area.

Input: Household composition information and health-related information (member age, sex, disease codes, etc.) and nutrition reference records.

Processing: Server performs numeric calculations to compute nutrition requirement information for each member by applying base nutrient values from reference tables, scaling by age, weight, or sex factors, and adjusting by disease-specific modifiers (e.g., reductions for sodium, sugar).

Output: Nutrition requirement information for each household member, represented as a structured data object and stored in memory and optionally persisted in the database.

### Step 5

Server assembles structured information that combines nutrition requirement information, household composition information, and health-related information.

Input: Nutrition requirement information and stored household composition and health-related records.

Processing: Server constructs a multi-field data structure representing all constraints and targets, then applies formatting templates to convert selected fields into a natural-language prompt sentence suitable for the generative AI model.

For example, server may create a prompt sentence:

“Generate a 7-day dinner menu for a family. One member is a child with a nut allergy and a preference for mildly spicy food. Another member is an elderly person with type 2 diabetes who requires low-sugar and low-salt meals. Use seasonal winter ingredients such as root vegetables.

Ensure each day's menu meets recommended nutritional guidelines, avoids nuts entirely, limits sugar and salt for the elderly member, and provides sufficient calcium and vitamin D for the child. Return the result as a list of days with dishes and ingredients.”

Output: Prompt sentence text and associated structured context, ready to be transmitted to the generative AI model.

### Step 6

Server sends the prompt sentence to the generative AI model using an inference API call.

Input: Prompt sentence and optional decoding parameters (e.g., maximum length, temperature, top-k).

Processing: Server encodes the prompt into tokens, invokes the generative AI model, and receives generated text as response data. The generative AI model internally performs neural network forward propagation through transformer layers to produce token probabilities and a sequence of output tokens.

Output: Response data representing a candidate menu description, in natural language or semi-structured text.

### Step 7

Server parses the response data to extract menu information.

Input: Natural-language or semi-structured response text from the generative AI model.

Processing: Server applies parsing rules, such as regular expressions or a lightweight parser, to identify days, dish names, and ingredient lists; server then maps these elements into a structured menu data object (e.g., list of days, each containing one or more dishes, each dish containing ingredient entries).

Output: Structured menu information stored in memory and optionally in the database.

### Step 8

Server validates the structured menu information against health-related constraints.

Input: Structured menu information, household health-related information (allergies, intake restrictions), and nutrition requirement information.

Processing: Server compares each ingredient in the menu with allergy and restriction lists to detect prohibited items, and estimates nutrient totals for each day by aggregating ingredient-level nutrient values obtained from nutrition reference tables. Server flags days or dishes that violate constraints (e.g., dishes with nuts, days exceeding sodium limits).

Output: Validated menu information and a list of violations, if any.

### Step 9

Server regenerates or refines menu parts when violations are detected.

Input: List of violations, original prompt sentence, and menu information.

Processing: Server constructs an additional prompt sentence that describes the violations and specifies corrections, such as:

“Regenerate the menu for day 3. The previous menu included nuts. Please strictly avoid any nut ingredients while maintaining the same nutritional targets.”

Server sends this additional prompt sentence to the generative AI model, receives a corrected segment, and merges the corrected segment into the existing menu.

Output: Corrected and constraint-compliant menu information stored in structured form.

### Step 10

Server aggregates ingredient information across the corrected menu to generate ingredient list information.

Input: Corrected menu information with dish-level ingredient entries.

Processing: Server groups ingredients by normalized name, sums quantities, and performs unit conversion (e.g., grams to kilograms). Server produces a list of required ingredient amounts per ingredient.

Output: Ingredient list information specifying total required amount for each ingredient.

### Step 11

Server queries external product information providing devices to map required ingredients to purchasable products.

Input: Ingredient list information and external product catalog data accessible via an API.

Processing: Server sends product lookup requests containing ingredient names and properties, receives product candidates with identifiers, sizes, and prices, and selects one or more products per ingredient based on matching criteria and quantity requirements.

Output: Ingredient candidate information associated with each required ingredient amount, and an updated ingredient list information including specific product identifiers.

### Step 12

Server constructs order information and determines a delivery schedule.

Input: Ingredient list information with product identifiers, user delivery-preference time-band information, and delivery-slot information retrieved from an external goods-delivery management device.

Processing: Server compares preferred time bands with available delivery slots, selects compatible slots, and assigns ordered products to slots. Server packages product identifiers, quantities, address, and selected slots into order records.

Output: Order information messages and finalized delivery schedule data stored in the database and ready to be transmitted to the delivery management device.

### Step 13

Server transmits order information to the external goods-delivery management device.

Input: Order information and delivery schedule data.

Processing: Server formats the order as a request message, sends it via a network protocol, and receives confirmation or error responses; server updates local status fields with the received confirmation identifiers.

Output: Confirmed orders and status records indicating that required ingredients will be delivered according to the schedule.

### Step 14

Server generates cooking procedure data based on the finalized menu and nutrition requirement information.

Input: Corrected menu information (dishes and ingredients) and nutrition requirement information.

Processing: Server assigns cooking styles, preparation times, and default temperatures to dishes by consulting a recipe template database; server produces step descriptions outlining sequence of operations (e.g., chopping, heating, stirring).

Output: Initial cooking procedure data objects for each dish.

### Step 15

Server refines cooking procedure data by using the generative AI model with device-capability constraints.

Input: Cooking procedure data, device-capability information (maximum temperature, supported operations), and health-related constraints.

Processing: Server constructs a prompt sentence describing the dish, device capabilities, and constraints, such as:

“Generate a detailed, step-by-step recipe for ‘Chicken and root vegetable soup’ optimized for a multifunction cooking apparatus that supports heating, stirring at variable speeds, and timed operations. Use metric units. Specify exact times, temperatures, stirring speeds, and alerts for adding ingredients. The recipe must avoid dairy and nuts and must remain low in salt.”

Server sends the prompt sentence to the generative AI model, receives stepwise instructions, and merges them with existing procedure data.

Output: Enhanced cooking procedure data including precise times, temperatures, and stirring speeds.

### Step 16

Server transforms the enhanced cooking procedure data into structured data including operation parameters for a cooking apparatus.

Input: Enhanced cooking procedure data (natural-language steps with parameters).

Processing: Server parses the steps to extract numeric parameters (e.g., 95° C., 10 minutes, stir speed level 2), maps natural-language actions to predefined operation types (heat, stir, pause), and creates a structured representation for each step.

Output: Structured operation-parameter data associated with each step of the recipe.

### Step 17

Server generates a sequence of control commands from the structured operation-parameter data.

Input: Structured operation-parameter data for a recipe.

Processing: Server creates command records specifying operation type, target temperature, time duration, stirring speed, and sequencing constraints; server orders these commands according to recipe step order and dependencies.

Output: Ordered control-command sequence ready for transmission to the cooking apparatus.

### Step 18

Server transmits the control-command sequence to the cooking apparatus and initiates execution.

Input: Control-command sequence and apparatus communication parameters.

Processing: Server opens a communication channel (e.g., via a network socket), sends batch or incremental commands, and waits for acknowledgments (e.g., “command accepted”, “step started”).

Output: Commands stored in the cooking apparatus's control queue and an initial apparatus state indicating that cooking has started.

### Step 19

Cooking apparatus executes received commands and sends sensor data to the server; server receives sensor data and operation-state information.

Input: Sensor readings such as temperature values, elapsed time counters, and execution statuses, transmitted from the cooking apparatus.

Processing: Server logs incoming data with timestamps, aligns sensor readings with corresponding commands, and updates an internal representation of current cooking state.

Output: Updated cooking state, including comparisons between current sensor values and target parameters for each command.

### Step 20

Server evaluates the cooking state and generates additional control commands when needed.

Input: Current cooking state (sensor data vs. target parameters) and original control-command sequence.

Processing: Server computes error values (e.g., difference between actual and target temperature), checks thresholds (e.g., tolerance range, timeout), and determines whether adjustments are required. If required, server creates additional control commands such as extended heating time or modified temperature settings and appends or replaces commands in the execution queue.

Output: Adjusted control-command sequence and additional control-command messages transmitted to the cooking apparatus.

### Step 21

Server detects completion of cooking and informs the terminal.

Input: Operation-state information indicating the final step executed successfully, or an explicit completion notification from the cooking apparatus.

Processing: Server marks the recipe execution as complete in the database, stops sending commands, and generates a completion message containing dish name, completion time, and any deviations from original plan.

Output: Completion message sent to the terminal and stored completion status for the recipe.

### Step 22

Terminal displays cooking progress and completion to the user and allows feedback input.

Input: Progress updates and completion message from the server.

Processing: Terminal updates visual indicators of current step, remaining time, and final completion status; terminal provides controls for the user to rate the dish or comment on saltiness, doneness, or preference.

Output: User feedback data entered on the terminal and prepared for transmission to the server.

### Step 23

Terminal sends user feedback to the server; server stores feedback as learning data.

Input: User feedback, including ratings and textual comments, associated with a particular menu, recipe, and household profile.

Processing: Server links feedback to corresponding prompt sentences, response data, and execution logs and stores these associations as learning data; server updates internal prompt-generation parameters (e.g., adjusts emphasis on low-salt constraints) based on aggregated feedback statistics.

Output: Updated learning-data records and modified prompt-generation conditions that will influence subsequent prompt sentences for the generative AI model.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented meal planning and cooking support systems typically perform rule-based menu generation, static shopping list creation, and simple device control without deeply integrating real-time health constraints, dynamic supply conditions, and adaptive cooking control. In many such systems, nutritional calculation modules and menu generators operate on fixed templates or pre-defined rules, and any use of a machine learning model is limited to isolated recommendation logic. As a result, these systems suffer from several technical shortcomings. First, existing systems generally lack a unified architecture for representing health information, nutritional requirements, menus, and ingredient lists in a structured format that can be consistently consumed both by deterministic computation modules and by generative artificial intelligence models. Without such a representation and control flow, the system must duplicate data transformations at multiple layers, leading to increased processing overhead, inconsistent constraint enforcement, and difficulty in verifying that generated content satisfies medical and allergen constraints. This causes inefficiencies at the processor level, including redundant computation, fragmented memory usage, and increased latency when generating or updating meal plans. Second, conventional systems that attempt to employ large-scale generative models often send loosely defined natural language prompts to the models and then manually or heuristically post-process free-text outputs. This ad hoc prompting and parsing approach is error-prone and computationally inefficient. The processor must execute additional parsing routines, extra validation passes, and repeated model calls to correct constraint violations. Moreover, because prompt content is not systematically tied to internal data structures and device specifications, the generated outputs can be misaligned with nutritional constraints, allergen restrictions, and the capabilities of cooking apparatuses. This misalignment leads to frequent re-generation, unnecessary network calls to external models, and suboptimal use of processing and communication resources.

Third, in many systems, control of cooking apparatuses is decoupled from high-level meal planning and from any generative content. Cooking devices are often operated using static scripts or simple timers that do not leverage detailed, structured recipes derived from generative models and do not adapt in real time to sensor feedback. As a consequence, the processor controlling the apparatus cannot perform fine-grained feedback control based on coherent, machine-readable procedure data originating from upstream planning logic. This separation results in underutilization of sensor data, difficulty in automatically adjusting cooking parameters for individual health needs, and additional manual intervention by the user, all of which undermine the efficiency and reliability of the overall computer system.

Fourth, existing architectures do not provide a cohesive mechanism by which the processor can use generative artificial intelligence not only for high-level menu proposals but also for generating structured, device-specific cooking procedures and user-facing explanations in a coordinated manner. When different components separately invoke generative models with unrelated prompts and output formats, the system must perform additional adaptation, mapping, and consistency checking, which increases complexity and computational cost. The lack of a centralized prompt control strategy tied to internal structured data also makes it difficult to systematically reduce the number of model calls and to cache or reuse results.

Therefore, there is a need for an improved computer-implemented system that: (i) centrally manages structured representations of health information, nutritional requirements, menu plans, ingredient data, and cooking procedures; (ii) programmatically generates and controls prompt sentences to generative artificial intelligence models based on these structured data and device specifications; (iii) automatically verifies and corrects model outputs against explicit allergen, disease, and nutrition constraints; and (iv) integrates such verified outputs with a real-time feedback control loop for a cooking apparatus. Such a system can reduce redundant computation, improve consistency of constraint enforcement, lower latency in end-to-end meal planning and cooking workflows, and enhance the reliability and safety of automated cooking operations by more efficiently utilizing processing, memory, and communication resources.

The specific processing by the specific processing unit 290 of the data processing device 12 in Application Example 1 is realized by the following means.

The present invention provides a server comprising a processor configured to acquire, via a health information acquisition unit, structured health information of a care recipient including at least age, sex, health condition, allergy information, dietary preference, and special nutritional need; to calculate, by a nutrition information calculation unit, a nutritional requirement for the care recipient based on the acquired health information using a predetermined nutrition algorithm; to generate, by a menu information generation unit, structured meal plan information for the care recipient based on the calculated nutritional requirement and the dietary preference; to generate, by an ingredient information generation unit, ingredient procurement information by aggregating ingredient information included in the meal plan information over a planning period; to coordinate, by a procurement cooperation unit, ordering information and delivery schedule information with an external procurement service based on the ingredient procurement information; to generate, by a cooking procedure generation unit, structured cooking procedure information based on the meal plan information, the nutritional requirement, and an operating specification of a cooking apparatus, and to convert the structured cooking procedure information into control procedure information conforming to the operating specification; to control, by a cooking apparatus control unit, at least a heating mechanism, a stirring mechanism, and a sensor of the cooking apparatus in real time based on the control procedure information and on sensed cooking state information so as to perform feedback control of temperature, time, and operation state; to cooperate, by a generative artificial intelligence cooperation unit, with a generative artificial intelligence model by programmatically generating, based on internal structured data including the health information, the nutritional requirement, the meal plan information, and ingredient information, a prompt sentence expressed in natural language, inputting the prompt sentence into the generative artificial intelligence model, obtaining candidate menu information, candidate ingredient information, or candidate cooking procedure information from the generative artificial intelligence model as structured output, and verifying and correcting the structured output based on at least one of an allergen constraint, a disease constraint, and a nutrition constraint; and to control, by a prompt control unit, content and format of the prompt sentence supplied to the generative artificial intelligence model such that the prompt sentence includes at least one of a health state of the care recipient, a restricted ingredient, a required nutrient, period information, and the operating specification of the cooking apparatus, and such that structured output from the generative artificial intelligence model is directly convertible into internal data formats used by the menu information generation unit, the ingredient information generation unit, and the cooking procedure generation unit. This enables a technically improved computer-implemented meal planning and cooking control system in which the processor reduces redundant data transformations, decreases the number of generative model invocations, maintains consistency between internal structured data and natural-language prompts, enforces medical and allergen constraints at the system level, and integrates verified generative outputs into a real-time feedback control loop for the cooking apparatus, thereby improving processing efficiency, reliability, and responsiveness of the overall system.

The term “health information acquisition unit” refers to a functional component implemented by hardware and/or software that obtains, via an input interface, structured health-related information of a person, including at least age, sex, health condition, allergy information, dietary preference, and special nutritional need.

The term “health information” refers to structured data representing personal medical and physiological attributes, including at least age, sex, health condition, allergy information, dietary preference, and special nutritional need of a care recipient.

The term “nutrition information calculation unit” refers to a functional component implemented by hardware and/or software that calculates a nutritional requirement for a care recipient based on health information, by executing a predetermined nutrition algorithm using processing resources of a processor.

The term “nutritional requirement” refers to target values or ranges for intake of energy and nutrients, including at least calories and macronutrients and optionally micronutrients, that are computed for a care recipient based on health information and dietary guidelines.

The term “menu information generation unit” refers to a functional component implemented by hardware and/or software that generates structured meal plan information for a care recipient based on a nutritional requirement and at least one preference, such as a dietary preference or health constraint.

The term “meal plan information” refers to structured data describing a plurality of meals over a specified period, including at least identification of dishes, associated ingredients, and an association with a care recipient's nutritional requirement.

The term “ingredient information generation unit” refers to a functional component implemented by hardware and/or software that aggregates ingredient information associated with meal plan information and generates ingredient procurement information for ordering or purchasing food ingredients.

The term “ingredient information” refers to structured data identifying food items required to prepare dishes in a meal plan, including at least ingredient names, quantities, and optionally unit types and categories.

The term “ingredient procurement information” refers to structured data representing a consolidated list of food ingredients to be procured, including at least ingredient identifiers, required quantities, and optionally supplier-related information, for use in automated ordering and delivery scheduling.

The term “procurement cooperation unit” refers to a functional component implemented by hardware and/or software that interacts with at least one external procurement service to transmit ingredient procurement information and to coordinate ordering information and delivery schedule information of food ingredients.

The term “external procurement service” refers to a system or service, accessible via a communication network, that receives ordering information for food ingredients and manages purchase processing and delivery scheduling.

The term “ordering information” refers to structured data representing a request to purchase specified food ingredients from an external procurement service, including at least item identifiers, quantities, and destination information.

The term “delivery schedule information” refers to structured data indicating planned or adjusted delivery times or time windows for delivering ordered food ingredients to a designated location.

The term “cooking procedure generation unit” refers to a functional component implemented by hardware and/or software that generates structured cooking procedure information based on meal plan information, a nutritional requirement, and an operating specification of a cooking apparatus, and converts the cooking procedure information into control procedure information.

The term “cooking procedure information” refers to structured data describing a sequence of cooking steps, including at least step order, actions to be performed, and associated parameters such as target temperature and time.

The term “control procedure information” refers to structured data derived from cooking procedure information that is adapted to conform to an operating specification of a cooking apparatus and is usable as a basis for generating specific control commands for the cooking apparatus.

The term “operating specification of a cooking apparatus” refers to structured data describing capabilities, parameter ranges, modes of operation, and interface requirements of a cooking apparatus, including at least allowable temperature ranges, available operation modes, and controllable parameters.

The term “cooking instruction generation unit” refers to a functional component implemented by hardware and/or software that generates at least a start instruction, an operation instruction, and a parameter change instruction for a cooking apparatus based on control procedure information.

The term “start instruction” refers to control information that causes a cooking apparatus to initiate execution of a cooking operation or a cooking program.

The term “operation instruction” refers to control information that directs a cooking apparatus to perform a specific action, such as heating, stirring, or holding at a given temperature, during a cooking process.

The term “parameter change instruction” refers to control information that modifies at least one operating parameter of a cooking apparatus, such as a target temperature, a stirring speed, or a remaining cooking time, during execution of a cooking process.

The term “cooking apparatus control unit” refers to a functional component implemented by hardware and/or software that controls, in real time, at least a heating mechanism, a stirring mechanism, and a sensor of a cooking apparatus based on instructions and sensed cooking state information, and that performs feedback control of temperature, time, and operation state.

The term “heating mechanism” refers to a physical component of a cooking apparatus, such as a heater or heating plate, that increases or maintains the temperature of food or a cooking container under control of the cooking apparatus control unit.

The term “stirring mechanism” refers to a physical component of a cooking apparatus, such as a motor-driven agitator or mixer, that agitates or mixes food during cooking under control of the cooking apparatus control unit.

The term “sensor” refers to a physical sensing element or device associated with a cooking apparatus that detects at least one parameter related to cooking, such as temperature, time, weight, or movement, and outputs sensor information to the cooking apparatus control unit.

The term “cooking state information” refers to sensor data and status data acquired from a cooking apparatus during cooking, including at least temperature information, time information, and operation state information.

The term “generative artificial intelligence cooperation unit” refers to a functional component implemented by hardware and/or software that cooperates with a generative artificial intelligence model by generating prompt sentences expressed in natural language based on internal structured data, inputting the prompt sentences into the generative artificial intelligence model, obtaining structured candidate information from the generative artificial intelligence model, and verifying and correcting the candidate information based on at least one constraint.

The term “generative artificial intelligence model” refers to a machine-learned model configured to generate content in response to an input, where the content may include text, structured data, or other machine-readable information, and which is capable of responding to natural language prompt sentences.

The term “prompt sentence” refers to a natural language expression, optionally combined with embedded structured data, that is supplied as an input query or instruction to a generative artificial intelligence model to request generation of specific information.

The term “candidate menu information” refers to meal plan-related structured data output from a generative artificial intelligence model, including at least proposed dishes and associated information that may be subject to verification and correction.

The term “candidate ingredient information” refers to ingredient-related structured data output from a generative artificial intelligence model, including at least proposed ingredients and associated quantities or categories that may be subject to verification and correction.

The term “candidate cooking procedure information” refers to cooking-step-related structured data output from a generative artificial intelligence model, including at least proposed step sequences and parameters that may be subject to verification and correction.

The term “allergen constraint” refers to a condition that prohibits the inclusion of specified allergenic food items or ingredients in generated menu information, ingredient information, or cooking procedure information.

The term “disease constraint” refers to a condition that restricts nutritional values, ingredients, or cooking methods based on a health condition of a care recipient.

The term “nutrition constraint” refers to a condition that enforces compliance with nutritional requirement values or ranges when generating or selecting menu information, ingredient information, or cooking procedure information.

The term “prompt control unit” refers to a functional component implemented by hardware and/or software that controls content and format of prompt sentences supplied to a generative artificial intelligence model, such that the prompt sentences include designated information and that structured outputs are directly convertible into internal data formats used by other functional units.

The term “internal data format” refers to a predefined structured representation, such as a schema or data model, used by internal modules of the system to store and process information including health information, nutritional requirements, meal plan information, ingredient information, and cooking procedure information.

The term “structured output” refers to data generated by a generative artificial intelligence model in a format that includes explicit fields or structure, such as key-value pairs, records, or arrays, suitable for direct parsing and conversion into internal data formats.

In one embodiment, a server, a terminal, and a user cooperate to implement the claimed system. The server is realized by a general-purpose computing platform, such as a rack-mounted computer or a virtual machine instance in a cloud environment. The server includes at least one central processing unit (CPU), an optional graphics processing unit (GPU), a main memory, a non-volatile storage device, and a network interface. The CPU executes an operating system, such as a UNIX-based operating system, and middleware, such as a web server and an application framework.

The GPU, when present, executes a generative AI model by accelerating parallel linear algebra operations. The terminal is realized by a portable information processing device such as a smartphone, a tablet computer, or a personal computer including a display, an input device, and a communication interface. The user operates the terminal.

The server stores, in the non-volatile storage device, a plurality of software modules corresponding to the health information acquisition unit, the nutrition information calculation unit, the menu information generation unit, the ingredient information generation unit, the procurement cooperation unit, the cooking procedure generation unit, the cooking instruction generation unit, the cooking apparatus control unit, the generative artificial intelligence cooperation unit, and the prompt control unit. The server also stores, in a database management system such as a relational database, structured data tables for health information, nutritional requirements, menu plan information, ingredient information, cooking procedure information, control procedure information, device capability information, and logs of cooking apparatus operation.

The user uses the terminal to access a user interface provided by the server. The terminal executes a browser or a native application implemented, for example, using a graphical user interface toolkit, and displays input fields for age, sex, health condition, allergy information, dietary preference, and special nutritional need of a care recipient. The terminal transmits, via a network, the input data as structured records, such as key-value pairs, to the server using a communication protocol such as HTTPS. The server receives the data, validates formats and ranges, and normalizes the content into a predefined internal data format. For example, the server maps textual disease names to internal disease codes and converts free-text allergy descriptions into normalized allergen identifiers using a dictionary table.

The server stores the normalized health information in a health information table. The server then activates the nutrition information calculation unit implemented as a software module using a programming language such as a high-level scripting language combined with numeric computation libraries such as vector and matrix processing libraries. The server reads the health information records and accesses a nutrition reference table stored in the database. The nutrition reference table includes, for various combinations of age, sex, and health condition, base values and coefficients for daily energy, macronutrients, and micronutrients.

The server performs arithmetic operations to compute a nutritional requirement record for each care recipient. For example, the server calculates a base energy requirement using a basal metabolic rate formula, applies adjustment coefficients based on activity level and disease conditions, and then derives target ranges for carbohydrates, fats, proteins, and selected vitamins and minerals. The server uses an algorithm that solves a constrained optimization problem, such as a linear or convex optimization, to ensure that nutritional requirement values satisfy multiple inequalities corresponding to disease constraints, allergen constraints, and diet style constraints. The nutrition information calculation unit stores the resulting nutritional requirement in a nutritional requirement table in the database.

The server executes the menu information generation unit to produce meal plan information. The server retrieves candidate dish records from a dish library table. Each dish record includes a dish identifier, a list of ingredients, per-serving nutrient values, possible texture levels, and preparation constraints. The menu information generation unit constructs a machine-readable representation of the current nutritional requirement, user preferences, and planning period (for example, number of days and meals per day). The server then invokes the generative artificial intelligence cooperation unit.

The server implements the generative artificial intelligence cooperation unit by calling a generative AI model hosted on a dedicated inference engine. In one embodiment, the generative AI model is a transformer-based neural network having an encoder-decoder or decoder-only architecture, with multiple self-attention layers, feed-forward layers, layer normalization, and residual connections. The model is trained on large-scale text and structured data and fine-tuned on domain-specific corpora, including nutrition guidelines, recipes, and device-oriented cooking instructions.

The server constructs a prompt sentence in natural language by concatenating template text with serialized structured data. For menu generation, the server creates, for example, the following prompt sentence:

“Act as a medical dietitian and menu planner. Based on the following profile: age 78, female, diabetes, nut allergy, swallowing difficulty requiring soft food, and the following daily nutrient targets: energy 1600 kcal, carbohydrate upper limit 180 g, protein target 60 g, increased calcium and vitamin D. Generate a 7-day meal plan with breakfast, lunch, and dinner. Avoid all nuts, keep sugar low, ensure each meal is easy to swallow, and include at least one high-calcium dish per day. Return the plan as a list of days and, for each meal, the dish name, short description, and approximate nutrients.”

The server, using the prompt control unit, ensures that the prompt sentence includes explicit fields for health state, restricted ingredients, required nutrients, and period information, and that it requests structured output. The server forwards this prompt sentence to the generative AI model via an application programming interface. The generative AI model receives the tokenized prompt, processes it through its layers, and outputs a sequence of tokens representing the response. The server decodes the tokens into text and then parses the text into a structured representation conforming to the internal data format for meal plan information.

The server verifies the generated meal plan using deterministic algorithms. The server inspects each dish, checks ingredients against an allergen table, and computes approximate daily nutrient totals by summing the nutrient values of included dishes and comparing them to the nutritional requirement ranges. If a constraint violation is detected, the server either modifies the plan using substitution rules stored in a rule table or constructs a second prompt sentence that explicitly requests replacements for problematic dishes, such as:

“Replace the following dishes that contain nuts or exceed the sugar limit, while keeping total energy and protein similar and maintaining soft texture: [list of dish names and reasons]. Propose alternative dishes that satisfy the constraints.”

By generating constraint-informed prompt sentences and requesting structured output, the server reduces the need for multiple unconstrained model calls and deep text parsing, thereby improving computational efficiency and reducing communication load. The server stores the final verified meal plan information in a meal plan table.

The server activates the ingredient information generation unit to compute ingredient procurement information from the meal plan. The server traverses each dish entry, extracts ingredient records, and aggregates quantities across the defined period. The server converts units to a standard unit system and maps generic ingredient identifiers to purchasable items by querying a product catalog table and, optionally, external procurement services via their application programming interfaces. The server can also construct a prompt sentence to request grouping and simplification of the list, for example: “Given the following 7-day meal plan and raw ingredient list, consolidate the ingredients into a shopping list grouped by category (vegetables, meat, fish, dairy, grains, condiments). Merge duplicate items, sum quantities, and output the result as a human-readable list with categories and items.”

The server parses and structures the model response and uses it to populate an ingredient procurement table. By delegating grouping and summarization to the generative AI model under strict structured-output requirements, the server reduces local algorithmic complexity while still performing deterministic constraint checks on quantities and categories, thereby improving the balance between model computation and conventional computation.

The server operates the procurement cooperation unit to synchronize the ingredient procurement information with external procurement services. The server constructs API requests including item identifiers, quantities, destination address, and desired delivery windows. The server receives proposed delivery options and selects an optimal schedule based on stored user preferences and cost metrics. The server stores the selected delivery schedule in a delivery schedule table and sends notifications to the terminal.

The server uses the cooking procedure generation unit to produce cooking procedure information tailored to the specific cooking apparatus available to the user. The server stores, in a device capability table, the operating specification of each cooking apparatus, including supported operation modes, permissible temperature ranges, stirring speed levels, maximum durations, and communication protocol details. The server reads dish entries from the meal plan and their associated ingredients, then creates a prompt sentence such as:

“You are a recipe generator for an automatic cooking appliance with the following capabilities: temperature range 40° C.-200° C., stirring speed levels 0-5, maximum continuous cooking time 120 minutes. Generate a step-by-step recipe for ‘soft simmered fish with vegetables’ using the following ingredients and quantities. The dish must have a soft texture suitable for swallowing difficulty and must not contain nuts. For each step, specify: step number, concise description, target temperature in ° C., duration in seconds, stirring speed level, and any safety note.”

The server sends this prompt to the generative AI model, obtains the stepwise procedure, and converts the textual output into a structured cooking procedure record. The server checks that all parameters fall within the device capability ranges and that all ingredients are used. The server then transforms the cooking procedure into control procedure information. For example, the server maps each step into low-level commands such as “PREHEAT”, “ADD_INGREDIENT”, “STIR”, and “HOLD”, and associates numeric parameters for temperature, duration, and stirring speed.

The server stores the control procedure information in a control procedure table. The cooking instruction generation unit, implemented as a software component, translates these entries into device-specific control messages. In one embodiment, the server uses a message queue protocol or a lightweight publish-subscribe protocol to send commands to the cooking apparatus via the terminal acting as a gateway. The terminal receives the control messages and transmits them to the cooking apparatus over a local communication interface, such as wireless communication or short-range wireless communication.

The cooking apparatus control unit, residing on the server or on an embedded controller in the cooking apparatus, uses the control procedure information to perform real-time feedback control. The cooking apparatus includes a heating mechanism, a stirring mechanism, and sensors such as temperature sensors and load sensors. The apparatus periodically transmits sensor readings and status flags to the server or terminal. The server compares the sensor readings with target values in the control procedure information. For example, the server executes a control loop that, at fixed intervals, computes an error between measured and target temperature and adjusts heater power according to a control law such as proportional-integral-derivative (PID) control. If the server detects that the measured temperature deviates beyond a predefined range, the server modifies subsequent control messages to increase or decrease heating duration or stirring intensity.

The server logs cooking state information and control decisions for later analysis. The server can also construct prompt sentences for generating user-facing explanations or summaries, for example: “Explain in simple terms what the cooking appliance is doing in the following sequence of steps, so that a caregiver can understand: [step list and parameters].”

The server uses the generative AI model to produce natural language explanations, which the terminal displays to the user. This integration of structured control data and explanatory text reduces the cognitive load on the user while keeping the control logic deterministic and device-specific. To implement the generative AI model, the server uses a trained neural network with a defined architecture. In one example, the model includes multiple transformer layers, each having multi-head self-attention with a specified number of heads, feed-forward layers with a specified hidden dimension, and learned positional embeddings. The model is trained using a supervised learning method in which the server or an offline training system minimizes a loss function such as cross-entropy between predicted tokens and target tokens, using stochastic gradient descent or a variant such as Adam. Training data includes paired examples of prompts and desired outputs for meal plans, ingredient lists, and cooking procedures. Fine-tuning is performed using domain-specific corpora and structured representations converted into textual forms. The server may also apply data augmentation, such as paraphrasing prompts or varying numeric targets within medical ranges, to improve robustness.

The server, by controlling the content and structure of prompt sentences and requiring structured outputs, uses the model in a way that is tightly coupled to internal data schemas. This non-conventional usage differs from mere natural-language chat and allows the server to reduce parsing errors, to directly map model outputs to database records, and to enforce validation rules systematically. As a result, the server decreases the number of model invocations and the amount of post-processing, thus reducing latency and computational load. The design of the prompt control unit as a mediator between internal data formats and natural language prompts constitutes an improvement to computer operation by optimizing the interaction pattern with an external or internal generative AI engine.

The terminal primarily handles user interaction and local communication with the cooking apparatus. The terminal displays views of health information, nutritional requirements, menus, ingredient lists, delivery schedules, and cooking statuses. The terminal renders graphical components based on structured data received from the server. When the terminal operates as a communication gateway to the cooking apparatus, the terminal maintains a local connection, forwards control messages from the server to the apparatus, and sends sensor readings from the apparatus back to the server. The terminal may also perform preliminary validation or buffering to smooth network interruptions.

The user, in realistic operation, interacts with the system by providing health information, confirming generated menus and ingredient lists, authorizing orders, and starting or stopping cooking operations. For example, the user can review a generated 7-day menu that includes soft meals for a person with a swallowing disorder and can approve the recommended ingredient order and delivery schedule. The user can then initiate a cooking sequence for a selected dish via the terminal, and the system will operate the cooking apparatus according to the generated control procedure.

In further embodiments, the server may employ different types of generative AI models, such as encoder-decoder models fine-tuned to produce JSON-like structures for control procedures, or models with specialized heads for predicting numeric parameters. The server may vary the optimization algorithm used in the nutrition information calculation unit or may modify the structure of the meal plan information to accommodate additional constraints, such as budget or cultural preferences. The device capability table may be adapted to other types of cooking apparatus, including ovens, steamers, or multi-chamber devices, and the cooking apparatus control unit may incorporate additional control algorithms, such as model predictive control, when higher precision is required.

By combining deterministic computation modules, structured data representations, a controlled interaction with a generative AI model through carefully designed prompt sentences, and a feedback control loop for physical cooking apparatuses, the server achieves technical effects beyond automation of human tasks. The server reduces redundant data transformation, lowers communication overhead with the generative AI model, improves accuracy of constraint enforcement, and enhances responsiveness and stability of cooking control. This integrated architecture provides a concrete technological improvement in the field of computer-implemented meal planning and automated cooking control.

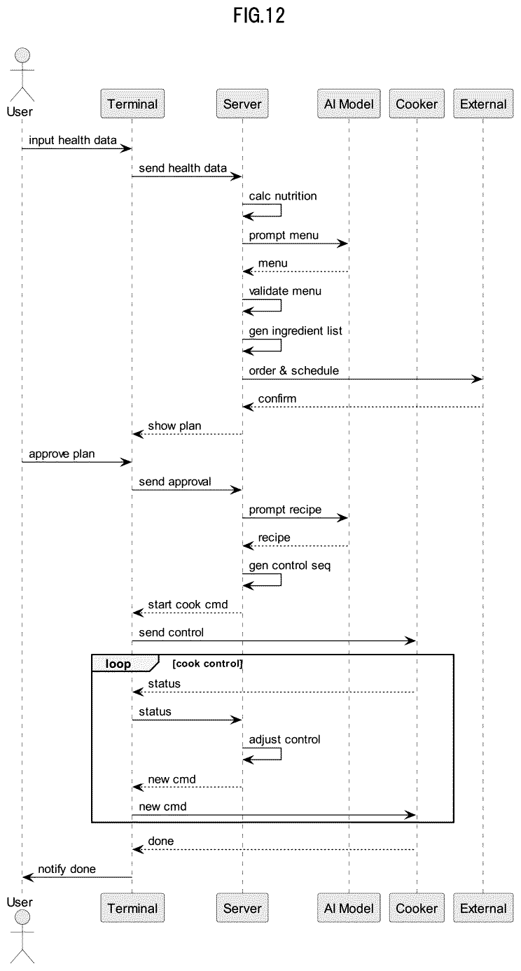

The following describes the processing flow using FIG. 12.

### Step 1

User uses the terminal to open the caregiving meal-support application and selects a target care recipient.

Terminal displays input screens for age, sex, health condition, allergy information, dietary preference, and special nutritional need.

Input: Raw user-entered values (text, selections, numbers).

Terminal performs client-side checks (for example, confirming that age is numeric and required fields are not empty) and normalizes formats (for example, trimming spaces, converting full-width characters to half-width).

Terminal transmits the normalized input to the server as structured key-value data over HTTPS.

Output: Structured health information request message sent to the server.

### Step 2

Server receives the health information request and parses the structured data.

Input: Structured key-value pairs representing age, sex, health condition, allergies, preferences, and special nutritional needs.

Server validates data types and ranges (for example, checking that age is within a plausible range and that disease names match known codes in a disease master table).

Server converts textual entries into internal codes (for example, mapping “diabetes” to a disease code and “nut allergy” to a specific allergen code) using lookup tables in a database.

Server stores the converted record into a health information table with a unique care recipient identifier.

Output: Normalized health information record stored in the database and an internal identifier returned to the application logic.

### Step 3

Server activates the nutrition information calculation unit to compute nutritional requirements.

Input: Normalized health information record obtained from the health information table.

Server loads nutrition reference data, such as standard recommended nutrient values per age/sex group and disease-specific constraints, from a nutrition reference table.

Server performs arithmetic operations to compute base energy needs (for example, applying a basal metabolic rate formula) and adjusts these values using coefficients for activity level and disease conditions.

Server uses vector and matrix operations to derive per-day target values for energy and macro-/micro-nutrients, then applies constraint checks to ensure that all values lie within medically acceptable bounds.

Server stores the resulting nutritional requirement record in a nutritional requirement table indexed by the care recipient identifier.

Output: Structured nutritional requirement record containing target ranges for calories and nutrients.

### Step 4

Server invokes the menu information generation unit to prepare inputs for the generative AI model.

Input: Nutritional requirement record, care recipient preferences, and planning period information (for example, 7 days, 3 meals per day).

Server retrieves candidate dish templates from a dish library table, including dish names, ingredient lists, per-serving nutrient values, and texture levels.

Server aggregates these internal data into a structured context and generates a prompt sentence in natural language.

Server, via the prompt control unit, constructs a detailed prompt such as:

“Act as a medical dietitian and menu planner. Based on the following profile: age 78, female, diabetes, nut allergy, swallowing difficulty requiring soft food, and the following daily nutrient targets: energy 1600 kcal, carbohydrate upper limit 180 g, protein target 60 g, increased calcium and vitamin D. Generate a 7-day meal plan with breakfast, lunch, and dinner. Avoid all nuts, keep sugar low, ensure each meal is easy to swallow, and include at least one high-calcium dish per day. Return the plan as a list of days and, for each meal, the dish name, short description, and approximate nutrients.”

Server tokenizes this prompt for the generative AI model.

Output: Tokenized and structured prompt sentence ready for submission to the generative AI model.

### Step 5

Server sends the tokenized prompt to the generative AI model and obtains a first candidate menu plan.

Input: Tokenized prompt sentence representing health constraints, nutrient targets, and planning period.

Server calls an inference API or local inference engine that executes a transformer-based generative AI model.

Server receives a sequence of output tokens from the model and decodes them into text.

Server parses the textual output into structured menu plan data, mapping days, meals, and dishes into database-compatible records with dish names, descriptions, and approximate nutrient values.

Server stores the candidate menu plan in temporary tables for validation.

Output: Structured candidate menu plan consisting of multiple dishes per day with associated metadata.

### Step 6

Server validates and corrects the candidate menu plan against constraints.

Input: Structured candidate menu plan, nutritional requirement record, allergen table, and disease constraint rules.

Server iterates over each dish and checks ingredient lists against the allergen table to detect forbidden ingredients.

Server sums nutrients per day by aggregating per-dish nutrient values and compares these sums with the nutritional requirement ranges for energy, carbohydrates, fats, proteins, and key micronutrients.

Server flags dishes or days that violate allergen or nutritional constraints.

If violations are detected, server either replaces specific dishes using a rule-based substitution table or forms a new corrective prompt sentence, such as:

“Replace the following dishes that contain nuts or exceed the sugar limit while keeping total energy and protein similar and maintaining soft texture: [dish list and reasons]. Propose alternative dishes that satisfy the constraints.”

Server resubmits this corrective prompt to the generative AI model and updates the menu plan with replacement dishes.

Output: Verified and constraint-compliant meal plan information stored in a meal plan table.

### Step 7

Server generates ingredient procurement information from the verified meal plan.

Input: Verified meal plan listing dishes, ingredients, quantities, and the planning period.

Server traverses all menu entries, extracts ingredient identifiers and quantities, and aggregates quantities per ingredient across the planning period.

Server converts units into standard units (for example, grams or milliliters) using a unit conversion table.

Server optionally constructs a consolidation prompt sentence, such as:

“Given the following 7-day meal plan and raw ingredient list, consolidate the ingredients into a shopping list grouped by category (vegetables, meat, fish, dairy, grains, condiments). Merge duplicate items, sum quantities, and output the result as a human-readable list with categories and items.”

Server sends this prompt to the generative AI model, parses the response into a structured, category-grouped ingredient list, and merges it with internally computed quantities.

Server stores the consolidated ingredient procurement information in an ingredient procurement table.

Output: Structured ingredient procurement list with ingredient identifiers, quantities, and categories.

### Step 8

Server coordinates ordering and delivery with external procurement services using the procurement cooperation unit.

Input: Ingredient procurement list, user delivery preferences, and external service API credentials.

Server maps internal ingredient identifiers to procurable product identifiers using a product catalog table.

Server constructs order requests including product identifiers, quantities, destination address, and preferred time windows, and sends them to external procurement services via their APIs.

Server receives proposed delivery options (time slots, availability, and costs), evaluates them according to predefined rules, and selects optimal options.

Server saves confirmed ordering information and delivery schedule information in a delivery schedule table and generates notification messages.

Output: Confirmed orders and delivery schedules associated with each ingredient batch, and notifications ready to be sent to the terminal.

### Step 9

Terminal retrieves meal plans, ingredient procurement information, and delivery schedules from the server for user review.

Input: Structured meal plan records, ingredient lists, and delivery schedule data obtained from the server over HTTPS.

Terminal renders views for each day's menu, the consolidated ingredient list, and the scheduled deliveries.

Terminal allows the user to approve, modify, or cancel specific items or delivery windows using touch or pointer input.

Terminal sends any user modifications back to the server as updated structured records.

Output: User-approved meal plan and procurement configuration, and update requests transmitted to the server.

### Step 10

Server updates internal records based on user modifications and finalizes cooking procedure generation inputs.

Input: User-approved or edited meal plan information and delivery schedule adjustments from the terminal.

Server applies deltas to the meal plan table and delivery schedule table, revalidates constraints if necessary, and ensures consistency across modules.

Server then collects finalized dish and ingredient data along with the cooking apparatus specification from the device capability table.

Server prepares structured input for the cooking procedure generation unit, including dish compositions and device parameters such as temperature ranges and available modes.

Output: Clean, validated dish and device data bundles serving as inputs to the cooking procedure generator.

### Step 11

Server generates device-specific cooking procedures using a generative AI model.

Input: Finalized dish information, ingredient details, and cooking apparatus specification.

Server constructs a prompt sentence tailored to cooking control, for example:

“You are a recipe generator for an automatic cooking appliance with the following capabilities: temperature range 40° C.-200° C., stirring speed levels 0-5, maximum continuous cooking time 120 minutes. Generate a step-by-step recipe for ‘soft simmered fish with vegetables’ using the following ingredients and quantities. The dish must have a soft texture suitable for swallowing difficulty and must not contain nuts. For each step, specify: step number, concise description, target temperature in ° C., duration in seconds, stirring speed level, and any safety note.”

Server sends the prompt to the generative AI model and decodes the response into structured stepwise instructions.

Server validates the instructions by confirming that all numeric parameters stay within the device capability limits and that all required ingredients are used.

Server stores the validated cooking procedure information in a cooking procedure table.

Output: Structured cooking procedure records with step numbers, actions, temperatures, durations, and stirring speeds.

### Step 12

Server converts cooking procedures into control procedure information and low-level control sequences.

Input: Cooking procedure records and device communication specification.

Server maps each high-level step into low-level control commands such as “PREHEAT”, “SET_TEMPERATURE”, “ADD_INGREDIENT”, “SET_STIR_SPEED”, and “WAIT_DURATION”, each with specific parameter values.

Server sequences these commands according to step order and encodes them in a format suitable for transmission (for example, JSON messages or binary frames).

Server stores the resulting control procedure information in a control procedure table, indexed by dish and schedule.

Output: Encoded control sequences representing fully specified cooking programs for the apparatus.

### Step 13

User initiates cooking for a selected dish through the terminal.

Input: Displayed menu options and associated dish identifiers retrieved from the server.

User selects a dish and possibly specifies a start time or immediate execution.

Terminal sends a cooking start request to the server, including the dish identifier and timing preference.

Terminal transitions to a status view ready to display cooking progress.

Output: Cooking start request message sent to the server, awaiting control commands.

### Step 14

Server orchestrates the transmission of control commands to the cooking apparatus via the terminal.

Input: Cooking start request, control procedure information, and the network address of the cooking apparatus or terminal gateway.

Server loads the relevant control sequence from the control procedure table and segments it into logical packets.

Server initiates a control session with the terminal designated as the gateway and sends the first set of commands (for example, preheat instructions and initial timing).

Terminal receives the control packets, establishes or confirms a communication link with the cooking apparatus, and forwards the control commands using the appropriate local protocol.

Output: Control commands delivered to the cooking apparatus to start and guide the cooking process.

### Step 15

Cooking apparatus executes commands and reports cooking state information.

Input: Low-level commands such as “SET_TEMPERATURE 180° C.”, “SET_STIR_SPEED 3”, and “WAIT 120 seconds”.

Cooking apparatus activates its heating mechanism, sets stirring speed, and tracks remaining time according to the received commands.

Cooking apparatus measures temperature and other sensor values at specified intervals and encapsulates these readings in status messages.

Terminal receives the status messages and relays them to the server.

Output: Periodic sensor readings and status updates (cooking state information) sent to the server.

### Step 16

Server performs real-time feedback control based on cooking state information.

Input: Cooking state information including current temperature, elapsed time, and motor state, and expected values from the control procedure information.

Server computes the difference between target and measured temperature and evaluates whether the system is within acceptable tolerance.

Server applies a control algorithm, such as PID control, to determine adjustments to heater power or stirring speed and generates new parameter change instructions if necessary.

Server also checks for abnormal conditions, such as excessive temperature deviations or stalled stirring, and, if detected, generates emergency stop or safe-mode commands.

Server transmits updated control commands to the cooking apparatus through the terminal.

Output: Adjusted control commands and, if needed, stop or safety commands sent to the cooking apparatus.

### Step 17

Terminal presents real-time cooking status and notifications to the user.

Input: Status updates and event notifications from the server, including current step, remaining time, temperature, and anomalies.

Terminal updates the user interface to show current cooking step, progress bars, and warnings or completion messages.

Terminal may display natural-language explanations generated previously by the server using a prompt such as:

“Explain in simple terms what the cooking appliance is doing in the following sequence of steps, so that a caregiver can understand: [step list and parameters].”

User monitors the status and may optionally send manual overrides (pause, extend time, adjust temperature) through the interface.

Output: Visual feedback to the user and, if overrides are used, override commands sent back to the server.

### Step 18

Server finalizes the cooking session and logs data for future optimization.

Input: End-of-cooking state from the apparatus, any user override commands, and final sensor traces.

Server confirms that the final cooking steps are completed, sends a completion notification to the terminal, and stops sending new control commands.

Server writes a session log including used cooking procedure, control sequence, sensor history, anomalies, and overrides into a log table or time-series database.

Server may later use these logs to adjust internal rules, refine nutritional algorithms, or improve future prompts to the generative AI model.

Output: Completed cooking session record and notifications of completion to the terminal, enabling continuous refinement of the system's performance.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented meal planning and cooking assistance systems have primarily focused on static conditions such as demographic information, fixed health constraints, and inventory or price data. In such systems, a processor typically computes nutritional targets from stored health data and generates menus or shopping lists using fixed rule sets or simple optimization logic. Even where machine learning or generative AI models are employed, these systems generally accept only static, pre-defined prompt inputs (for example, age, allergies, and calories) and do not dynamically adapt the prompts or downstream control logic based on a user's real-time emotional state or longitudinal feedback. As a result, the generated outputs may be nutritionally adequate but often fail to reduce practical user burden in situations of high fatigue or stress, or to reliably increase user satisfaction over time.

From the viewpoint of computer technology, existing systems also exhibit architectural and algorithmic limitations. First, emotion information, when used at all, is typically processed in a siloed manner, without a unified, multimodal pipeline in which a processor fuses audio, image, and text signals into a normalized emotional parameter space that is consistently reused across menu generation, ingredient selection, delivery scheduling, and appliance control. This fragmented handling of input data leads to brittle behavior, limited robustness, and inefficient use of computational resources, because each downstream module either ignores emotion information or re-implements its own narrow inference logic.

Second, existing systems do not exploit the control capabilities of modern generative AI models in a deeply integrated way. While a processor may call a large language model once with a static prompt to obtain a menu or recipe, the processor does not treat the prompt itself as an adaptive control parameter that can be programmatically regenerated, iteratively refined, and constrained using structured state (such as computed nutrient targets and emotional parameters). Consequently, the system cannot enforce hard nutritional constraints, cannot systematically bias the menu toward reduced user workload based on real-time stress or fatigue, and cannot guarantee stable convergence of generated outputs toward user-specific objectives.

Third, conventional systems typically separate high-level decision making (menu and recipe) from low-level device control (cooking apparatus commands) in a way that is manually bridged. A processor may convert a fixed recipe to simple commands, but it does not parse a generative AI model's output into a structured, machine-interpretable control sequence with explicit, numeric temperature and time parameters and clear separation between “user operations” and “automatic device operations.” Nor does the processor implement closed-loop feedback control where sensor data (temperature, time, image signals) from the cooking apparatus is used to dynamically adjust generated commands. This lack of end-to-end integration results in rigid workflows, susceptibility to cooking failures, and inefficient resource usage at the computing and device-control layers.

Fourth, conventional architectures are not designed to treat user feedback as training data for the entire computational pipeline. Satisfaction ratings, when collected, are rarely linked back to the emotional parameters, prompt templates, and selection heuristics used by the system, and are not systematically exploited to update prompt-generation logic or weighting functions for ingredient-form selection. As a result, the system cannot computationally “learn” from failures or successes in a structured fashion, and cannot improve its generative AI prompts or device-control strategies in a data-driven, automated way.

Accordingly, there is a need for an improved computer-implemented system and server technology in which a processor: (i) unifies multimodal emotion estimation into a machine-usable emotional parameter space; (ii) programmatically generates, evaluates, and iteratively refines prompt sentences for generative AI models based on nutritional targets and emotional parameters; (iii) maps generative AI outputs into structured control commands for a cooking apparatus with closed-loop sensor-based adjustments; and (iv) performs continual learning by updating calculation logic, prompt templates, and ingredient-selection weights using user feedback. Such a system should provide an improved technical effect in the operation of the computer itself, including more efficient and reliable use of generative AI models, more robust pipeline orchestration, and enhanced resource utilization in automatic cooking control.

The specific processing by the specific processing unit 290 of the data processing device 12 in Example 2 is realized by the following means.

The present invention provides a server comprising a processor and a memory storing instructions that, when executed by the processor, cause the processor to acquire and store structured health state data for household members, compute required nutrient amounts by referencing nutrition science information in the memory, estimate emotional parameters by processing multimodal user input data through a machine learning model or a generative AI model, programmatically generate and iteratively refine prompt sentences for a generative AI model based on the required nutrient amounts, the emotional parameters, and context information, parse outputs from the generative AI model into structured menu candidates and ingredient lists, select ingredient forms and delivery schedules using optimization logic that is explicitly controlled by the emotional parameters, generate recipe-generation prompt sentences that include explicit numeric control constraints and role separation between user operations and automatic device operations, convert generative AI-generated recipes into machine-interpretable control command sequences for a cooking apparatus, perform closed-loop adjustment of those command sequences using sensor data from the cooking apparatus, and record and utilize user satisfaction feedback to update at least one of nutrition calculation logic, prompt templates, and selection weights for ingredient forms. This enables an integrated, computer-implemented control architecture in which the processor dynamically coordinates emotion estimation, prompt sentence generation for the generative AI model, nutritional computation, ingredient selection, delivery scheduling, and appliance control in a closed feedback loop, thereby improving the technical performance and reliability of the generative AI-driven meal planning and automatic cooking pipeline, reducing computational waste from unsuitable generative outputs, enhancing robustness of device control through sensor-based adjustments, and allowing the server to adapt its internal algorithms over time based on accumulated feedback to more efficiently satisfy user-specific constraints and preferences.

The term “health state data” refers to digital data representing physical or medical attributes of one or more individuals, including at least age, sex, disease information, allergy information, food preference information, and special nutritional needs, which is used as an input for nutritional computation and menu generation.

The term “nutrition science information” refers to data stored in an information storage device that describes recommended nutrient intakes and food composition values, including recommended daily or per-meal amounts of energy and nutrients such as protein, fat, carbohydrate, vitamins, and minerals, for one or more age, sex, or health-condition groups.

The term “required nutrient amounts” refers to computed numerical target values for energy and one or more nutrients that a household member should consume during a specified period, such as per day or per meal, based at least on the member's health state data and the nutrition science information.

The term “nutrition balance calculation function” refers to program logic executed by a processor to reference nutrition science information and calculate, from health state data, the required nutrient amounts for each individual, including the application of rules or algorithms that modify nutrient targets according to disease information or special conditions.

The term “audio data” refers to a digital representation of acoustic signals corresponding to speech or sounds produced by a user, which can be processed by a computing device to perform speech recognition and acoustic feature extraction.

The term “image data” refers to a digital representation of at least one still image or video frame depicting at least a part of a user, such as a face or upper body, which can be processed by a computing device to perform detection or classification of facial expressions or other visual features.

The term “text data” refers to digital character data representing natural language text, including text directly input by a user via an input interface and text obtained by converting audio data into text using speech recognition processing.

The term “machine learning model” refers to a computational model generated by a learning algorithm based on training data, such as a classification model, regression model, or sequence model, that is configured to infer at least one target variable, such as an emotional state, from input features.

The term “generative AI model” refers to an artificial intelligence model, including a large language model, configured to generate natural language text or structured data in response to an input prompt sentence, and usable via an inference environment or application programming interface.

The term “emotional state” refers to a psychological condition of a user at a point in time, including one or more affective dimensions such as stress, fatigue, joy, sadness, tension, relaxation, or celebration mood.

The term “emotional parameters” refers to normalized data values, such as numerical scores or categorical labels, that represent an estimated emotional state of a user and include at least a stress level and a fatigue level and optionally further include a positivity score or event mood.

The term “emotion estimation function” refers to program logic executed by a processor that processes at least one of audio data, image data, and text data using a machine learning model or a generative AI model to infer an emotional state and to output emotional parameters.

The term “family composition information” refers to data describing at least the number and roles of household members, such as parent, child, or elder, and optionally including identifiers and demographic attributes, which are used to determine menu scope and portion sizes.

The term “health state information” refers to structured data derived from health state data and stored in an information storage device, including disease, allergy, and special nutritional needs information associated with one or more household members.

The term “nutrition condition information” refers to data specifying one or more constraints or goals relating to nutrient intake, such as targeted ranges or upper limits for energy, carbohydrate, sodium, or other nutrients, derived from required nutrient amounts.

The term “preference information” refers to data describing likes and dislikes for foods or cooking styles of one or more users or household members, such as preferred flavors, disliked ingredients, or cuisine types.

The term “season information” refers to data representing a temporal or environmental context for a given date or time period, including at least season of the year and optionally including temperature, weather conditions, or related environmental attributes.

The term “event information” refers to data indicating whether a specific day corresponds to a special occasion, such as a holiday, birthday, anniversary, or celebration event, and optionally including event type and importance.

The term “prompt sentence” refers to a sequence of natural language text describing conditions, constraints, or instructions that is provided as input to a generative AI model to control or influence the content or style of generated output.

The term “menu candidate” refers to a data structure representing a proposed set of dishes for a meal or meals, including at least dish names and dish types such as main dish, side dish, soup, or dessert, generated based on a prompt sentence and optionally refined according to nutritional and emotional criteria.

The term “menu generation function” refers to program logic executed by a processor to construct prompt sentences using required nutrient amounts, emotional parameters, season information, event information, and other constraints, to input the prompt sentences to a generative AI model, to receive generated text, and to parse the generated text into structured menu candidates.

The term “ingredient list” refers to data representing a collection of ingredient entries required to realize at least one menu candidate, each entry including at least an ingredient identifier, a quantity, and optionally a form or product identifier.

The term “ingredient form” refers to a particular physical or processing state of an ingredient, including at least fresh ingredients, pre-cut ingredients, marinated ingredients, frozen ingredients, or partially prepared ingredients.

The term “partially prepared ingredients” refers to ingredients that have undergone at least one preprocessing step, such as cutting, seasoning, or partial cooking, allowing completion of a dish in a shorter time or with fewer user operations compared to using fresh ingredients.

The term “external product provision device” refers to an information processing apparatus operated by a product or grocery service provider that exposes data on product stock status, prices, and attributes via a communication interface.

The term “ingredient list generation function” refers to program logic executed by a processor to extract required ingredients from menu candidates, query ingredient information and external product data, apply weighting based on emotional parameters, and select optimal ingredient forms and purchase sources to construct an ingredient list.

The term “user schedule information” refers to time-related data representing availability or preferences of a user, including at least at-home periods, working hours, or preferred delivery time slots, which is used for scheduling deliveries or cooking operations.

The term “ordering and delivery service device” refers to a remote information processing apparatus associated with a delivery or logistics service, configured to receive order information, arrange product shipment, and return order status information to the server.

The term “ordering and delivery cooperation function” refers to program logic executed by a processor to determine delivery dates and times based on an ingredient list and user schedule information, to create order information, and to communicate with an ordering and delivery service device to place and manage orders.

The term “cooking apparatus specification information” refers to stored data describing operational characteristics of a cooking apparatus, such as capacity, supported heating modes, allowable temperature range, available stirring modes, and communication protocol requirements.

The term “recipe-generation prompt sentence” refers to a prompt sentence tailored for a generative AI model that describes at least a target dish, constraints on user workload, explicit numeric heating temperature and heating time requirements, and a separation between user actions and automatic operations of a cooking apparatus.

The term “recipe generation function” refers to program logic executed by a processor to construct a recipe-generation prompt sentence using menu information, cooking apparatus specification information, and emotional parameters, to send the recipe-generation prompt sentence to a generative AI model, and to receive and store a generated cooking recipe.

The term “cooking recipe” refers to structured or semi-structured text describing, at a minimum, a sequence of cooking steps, ingredient input order, temperatures, times, and stirring or other control conditions required to prepare one or more dishes.

The term “cooking instruction function” refers to program logic executed by a processor to parse a cooking recipe, extract parameters such as step order, ingredient order, heating temperature, heating time, and stirring conditions, and convert the extracted parameters into a control command sequence corresponding to a cooking apparatus control protocol.

The term “control command sequence” refers to an ordered set of control messages or data structures specifying operations that a cooking apparatus is to perform, including at least start and stop times, target temperatures, durations, stirring operations, and optional mode settings.

The term “cooking robot control function” refers to program logic executed by a processor to transmit a control command sequence to a cooking apparatus, receive sensor and status data from the cooking apparatus, compute adjustments based on the received data, and transmit additional control commands to perform closed-loop control of automatic cooking.

The term “temperature sensor information” refers to digital data representing measured temperature values acquired from one or more temperature sensors embedded in or associated with a cooking apparatus.

The term “timer information” refers to digital data indicating time-related status of a cooking apparatus, including at least remaining time for current steps or elapsed time since the start of a cooking process.

The term “image information” refers to digital image data acquired from a camera associated with a cooking apparatus, representing at least a visual state of ingredients or food during cooking.

The term “terminal device” refers to a user-operated information processing apparatus, such as a mobile device, tablet, or personal computer, configured to display information received from a server and to transmit user inputs such as commands or feedback to the server.

The term “terminal cooperation function” refers to program logic executed by a processor to exchange data with a terminal device, including transmitting menu candidates, ingredient lists, order status information, and cooking recipes, and receiving cooking start instructions and satisfaction evaluation information.

The term “cooking start instruction” refers to digital data transmitted from a terminal device to a server indicating that a user has selected a specific menu and authorizes initiation of an associated cooking process by a cooking apparatus.

The term “satisfaction evaluation information” refers to user-provided feedback data regarding at least one of taste, convenience, or overall experience associated with a served meal or cooking process, including discrete ratings or textual comments.

The term “learning data” refers to data stored in an information storage device and used for training, updating, or fine-tuning models or decision logic, including at least associations between emotional parameters, menu candidates, selected ingredient forms, and satisfaction evaluation information.

The term “calculation logic of the nutrition balance calculation function” refers to one or more rules, algorithms, or parameter sets used by the nutrition balance calculation function to map health state data and nutrition science information to required nutrient amounts.

The term “template and constraint conditions of the prompt sentence” refers to structural patterns and parameterized fields used to generate a prompt sentence, together with explicit rules that restrict or guide the content of the prompt sentence, such as upper limits on cooking time or number of steps.

The term “weighting parameters used for selection between fresh ingredients and partially prepared ingredients” refers to numerical values or functions used by the ingredient list generation function to adjust relative preference scores for different ingredient forms such that selections can be biased toward or away from partially prepared ingredients based on emotional parameters or other conditions.

The term “operating state information of the cooking apparatus” refers to digital status data obtained from the cooking apparatus, including sensor readings, execution state of control commands, error codes, and progress indicators that describe current operation of each cooking step.

The term “warming period” refers to a time interval after primary cooking steps are completed during which the cooking apparatus maintains a dish at a safe serving temperature without performing substantial further cooking.

In one embodiment, a server, a terminal, and a cooking apparatus cooperate to implement the claimed system. The server is implemented as an information processing apparatus deployed on a general-purpose computing platform, such as a cloud computing environment that includes at least one central processing unit (CPU), a main memory, a secondary storage device, and a network interface. The server executes an operating system such as a server-type Linux OS, and runs on top of it an application execution environment such as a Python runtime, a Java runtime, or a script language runtime. The server further executes web server software such as a generic HTTP server, an application server such as a generic web application framework, and a relational database management system such as a general-purpose relational database.

The terminal is implemented as a user-operated information processing apparatus, such as a smartphone, a tablet computer, or a personal computer. The terminal executes a mobile operating system or a desktop operating system and runs either a dedicated native application or a web browser such as a general-purpose browser. The terminal includes at least a touch panel or pointing device, a keyboard or soft keyboard, a microphone, a camera, and a display unit. The cooking apparatus is implemented as an automatic cooking device that includes at least a heating mechanism, a stirring mechanism, one or more temperature sensors, an internal timer, and optionally an internal camera.

The cooking apparatus includes a network communication interface, such as a wireless network interface compliant with a generic wireless standard, or a wired local area network interface.

The server stores, in the secondary storage device, a set of software modules corresponding to functional units including a health state data acquisition module, a nutrition balance calculation module, an emotion estimation module, a menu generation module, an ingredient list generation module, an ordering and delivery cooperation module, a recipe generation module, a cooking instruction module, and a cooking apparatus control module, as well as a terminal cooperation module. The memory of the server holds data structures including a family information table, a health status table, a nutrition reference table, a food composition table, an emotion information table, a menu candidate table, an ingredient catalog table, an order status table, an appliance specification table, a cooking command table, a calendar information table, a user schedule table, and a feedback table.

The server uses the health state data acquisition module to receive health state data transmitted from the terminal. The terminal presents, on a graphical user interface, input fields for each family member's age, sex, body size, disease information, allergy information, food preferences, and special nutritional needs. The terminal validates these inputs by executing client-side validation logic that performs null checks, numeric range checks, and list-membership checks for enumerated values. The terminal then structures the validated data into a hierarchical data structure and transmits the data to the server via a secure HTTP-based protocol.

The server parses the received data, maps the fields to internal columns of the family information table and the health status table, and stores the data using prepared statements or an object-relational mapping library. The server thereby defines concrete database schemas for the entities and enforces constraints such as uniqueness and referential integrity. This structured storage enables efficient indexing and querying of health state data, which improves subsequent computation efficiency in the nutrition balance calculation module.

The server uses the nutrition balance calculation module to compute required nutrient amounts. The server stores, in the nutrition reference table, normalized entries indexed by age range, sex, physical activity level, and disease category. Each entry contains recommended daily intakes of energy and nutrients such as protein, fat, carbohydrate, and specific minerals and vitamins. The server also stores a food composition table that holds nutrient content per unit weight for various food items. The nutrition balance calculation module applies deterministic algorithms: the server selects the closest reference rows based on age and sex, and then adjusts certain nutrients according to disease fields.

For example, when the health status table indicates a diabetes-related flag, the server reduces allowable carbohydrate ranges and tightens acceptable glycemic load. When a hypertension-related flag is present, the server reduces maximum sodium values.

The server executes these adjustments by performing conditional branches and arithmetic operations realized in the program logic. The server then divides daily values into per-meal targets using an integer or floating-point division, storing these per-meal targets as required nutrient amounts for each family member in a nutrition target table. Because these required nutrient amounts are stored in normalized numeric form, the server can perform efficient vectorized comparisons with nutrient totals that the server later calculates for menu candidates. This structured representation of targets permits faster constraint-checking compared to ad hoc string-based rules, thereby improving processing speed and enabling tighter nutritional control.

The server uses the emotion estimation module to estimate emotional parameters. The terminal acquires multimodal user inputs: the microphone records a time series of audio samples, the camera acquires an image or a sequence of images of the user's face, and the graphical user interface collects free-text input describing subjective feelings. The terminal converts these to digital formats (e.g., audio waveforms, encoded images, and character strings) and transmits them to the server.

The server performs several signal-processing and inference steps. For audio data, the server uses a digital signal processing library to compute time-frequency features such as Mel-frequency cepstral coefficients (MFCCs), pitch contours, and energy profiles. For image data, the server uses a general-purpose image processing library to detect facial regions and normalizes the images by cropping and resizing. For text data, the server tokenizes the text using a natural language processing library and produces sequences of token identifiers.

The server feeds these features into at least one neural network model. In one embodiment, the server uses a convolutional neural network (CNN) for facial expression classification, a recurrent neural network (RNN) or transformer-based encoder for audio emotion classification, and a transformer-based language model for text-based emotion classification. Each model has parameters (weights and biases) that have been trained offline using supervised learning on labeled emotional datasets. The server stores these parameters in the secondary storage device and loads them into memory at runtime.

During inference, the server feeds the feature vectors into the neural networks and obtains output probability distributions across emotion classes such as “high stress,” “low stress,” “high fatigue,” “relaxed,” “positive mood,” and “celebration mood.” The server then applies a multimodal fusion algorithm. In one embodiment, the server uses a weighted averaging scheme in which each modality's confidence score weights its contribution. In another embodiment, the server uses a small fully-connected neural network that takes as input the concatenated emotion scores from audio, image, and text models and produces normalized emotional parameters. The server stores the resulting stress level, fatigue level, and additional parameters such as positivity score and event mood as standardized numeric values in the emotion information table.

This multimodal architecture improves robustness and accuracy of emotion estimation compared to a single-modality or rule-based approach. Because the server normalizes emotion outputs into numeric parameter vectors, the downstream modules can treat emotional parameters as part of a deterministic state space, enabling efficient algorithmic decisions and reducing the complexity of handling diverse raw sensor data. In particular, the server avoids re-running heavy models for each downstream decision, thereby reducing computational load and latency.

The server uses the menu generation module to construct prompt sentences for a generative AI model. The server retrieves, for each planning instance, the required nutrient amounts from the nutrition target table and the emotional parameters from the emotion information table, as well as family composition information, preference information, season information, and event information from the corresponding tables. The server uses a template-based text generation function that composes a prompt sentence by inserting numeric values and textual descriptors into a predefined structure. For example, the server generates a prompt sentence of the form:

“Based on the following conditions, propose 3 dinner menu options for today.

### [Family Composition and Health Status]

Female, 40s (mother): mild hypertension
Male, 40s (father): no specific disease
Boy, 10 years old (child): in growth period, dairy allergy
Female, 70s (grandmother): diabetes, prefers low salt

### [Nutritional Conditions]

The child should have menus rich in calcium and vitamin D.
The grandmother should have menus that are low in carbohydrates and low in salt, and easy to digest.

### [Preferences]

All family members: prefer less spicy food, like sweet desserts.

### [Current User Emotional State]

The user (mother) is very tired from work and has a high stress level.
Total cooking time should be within 30 minutes, with as few steps as possible and minimal dishes to wash.

### [Other Conditions]

The current season is winter, and at least one dish should be warming for the body.

Please output 3 menu sets (Japanese or Western style) that satisfy all the above conditions, in Japanese.”

The server transmits this prompt sentence to a generative AI model via an HTTP-based API. The generative AI model is implemented as a large language model deployed on an external inference service. The server sends the prompt sentence along with generation parameters such as maximum token length, temperature, and target language. The server receives text output describing multiple menu options.

Instead of treating the generative AI output as final, the server parses the output into a structured menu candidate representation and then computes actual nutrient totals by mapping dish names to standard recipes stored in the recipe information table and summing relevant nutrients using the food composition table. The server compares the calculated nutrient totals with the required nutrient amounts. When discrepancies exceed predetermined thresholds, the server programmatically adjusts the constraints in the prompt sentence. For example, if sodium content exceeds a target, the server augments the prompt with an explicit instruction such as “Ensure that sodium content remains below [numeric value] per person” or “Avoid high-salt ingredients such as certain pickles and processed meats.” The server re-generates the prompt sentence and re-invokes the generative AI model. This iterative loop constitutes a non-conventional control strategy, where the server treats the prompt sentence as a tunable control parameter, and uses deterministic, numeric checks on nutritional constraints to guide multiple generative attempts. This approach improves the reliability of generative AI outputs while reducing manual trial-and-error and enabling stable convergence toward feasible menus.

The server uses the ingredient list generation module to convert menu candidates into concrete ingredient lists. The server aggregates ingredients across selected recipes and consults an ingredient catalog table that describes, for each base ingredient, available forms such as fresh, pre-cut, marinated, and frozen. The server associates each ingredient form with metadata including estimated preparation time savings, cost, and storage requirements. The server then applies an optimization algorithm that uses emotional parameters as explicit decision variables. For example, when the fatigue level parameter exceeds a threshold, the server increases the weight assigned to time-saving forms in a cost function such as:

total_score=α·normalized_cost+β·normalized_preparation_burden,
where β is increased as a function of fatigue level. The server calls external product provision devices via their APIs to obtain real-time price and stock information, and populates candidate product lists for each ingredient form. The server evaluates each candidate by computing total_score and selects the product with minimum score.

This optimization algorithm is non-trivial and distinct from manual or rule-of-thumb selection. The server uses numeric emotional parameters to drive the choice of ingredient forms, which directly affects both computational decisions (fewer steps for the user) and physical resource usage (more processed ingredients at appropriate times). Because the server systematically ties emotional parameters to weight adjustments in the cost function, the server achieves improved user workload reduction and stable cost control, and also reduces recomputation by caching intermediate scores for recurring menus under similar emotional conditions.

The server uses the ordering and delivery cooperation module to schedule deliveries. The server reads user schedule information and order constraints from the database and computes candidate delivery windows that intersect user availability intervals. The server also incorporates emotional parameters into scheduling logic. For example, when stress level is high on a given evening, the server can schedule only minimal or ready-to-eat ingredients for that evening and schedule more complex ingredient deliveries for days when the emotional parameters indicate lower stress. The server constructs order messages that include product identifiers, quantities, and delivery time slots, and transmits them to external ordering and delivery service devices using standardized web service interfaces. The server receives order confirmation messages containing order identifiers and status, and updates the order status table accordingly.

The server uses the recipe generation module to create detailed recipes suitable for execution by the cooking apparatus. The server obtains cooking apparatus specification information from the appliance specification table, including capacity limits, supported heating modes, permitted temperature ranges, and stirring capabilities. The server also reads the selected menu and the emotional parameters. The recipe generation module composes a recipe-generation prompt sentence that instructs the generative AI model to produce a recipe with explicit numeric parameters and clearly separated roles. For example, the server generates a prompt sentence such as: “For the dish ‘Chicken and Root Vegetable Soy Milk Style Soup (dairy-free),’ generate a recipe suitable for cooking with a home automatic cooking robot.

### Conditions

The user is very tired from work, so user operations must be minimized. User operations should be limited to washing ingredients, roughly cutting them, and putting them into the robot pot.
The robot must be able to automatically control heating temperature, heating time, and stirring timings, so specify numerical values for temperature (° C.) and time (minutes) for each step.
The child has a dairy allergy, so do not use any dairy products such as milk or cheese. Use alternatives such as soy milk.
The grandmother is elderly, so ingredients should be cooked until soft and easy to eat.

Write the procedure with numbered steps, and for each step clearly separate ‘User operation’ and ‘Robot automatic operation’ in the description.”

The server sends this recipe-generation prompt sentence to the generative AI model and receives a text recipe. The server then executes parsing logic that uses pattern matching and rule-based parsing to extract the structure of the recipe. The server identifies numbered steps, segments each step into “User operation” and “Robot automatic operation,” and extracts numeric values for temperatures, times, and stirring instructions. The server forms an intermediate representation where each step is encoded as a record with fields such as step identifier, user action description, robot mode, target temperature, duration, and stirring interval.

The server uses the cooking instruction module to translate this intermediate representation into a control command sequence compatible with the communication protocol of the cooking apparatus.

The server maps each robot-mode field to a command type, assigns numeric parameters to command fields, and orders the commands according to the step identifiers and dependency rules. The server stores the resulting control command sequence in the cooking command table.

The server uses the cooking apparatus control module to transmit the control command sequence to the cooking apparatus. The server uses a bidirectional communication channel, which may be an HTTP-based API, a WebSocket connection, or a message-oriented middleware protocol. The server initiates execution upon receiving a cooking start instruction from the terminal. During execution, the cooking apparatus periodically sends temperature sensor information, timer information, and optionally image information to the server. The server interprets temperature curves and time progress and, in some embodiments, also processes image information using computer vision techniques to detect undercooked or overcooked states.

The server implements a closed-loop control algorithm. For each step, the server compares the measured temperature against the target temperature and evaluates whether heating has reached a steady state within a predetermined tolerance. If the server detects that the measured temperature remains below target for longer than an allowed threshold, the server computes an extension of heating time or an adjustment of target temperature within safety constraints defined in the appliance specification information. The server generates additional control commands to extend or adjust heating and transmits these commands to the cooking apparatus. This closed-loop scheme reduces cooking errors and variability due to disturbances such as ingredient mass differences or environmental temperature changes, thereby improving the reliability and technical performance of the automatic cooking.

The server uses the terminal cooperation module to provide information to the user and to receive feedback. The server transmits menu candidates, ingredient lists, order statuses, and summary recipe information to the terminal using structured data. The terminal renders these as interactive displays that allow the user to select a menu and to issue a cooking start instruction. After the cooking is completed, the server notifies the terminal, and the terminal prompts the user to provide satisfaction evaluation information in the form of ratings and optional comments. The server stores the feedback data in the feedback table.

The server executes a learning process that uses the stored feedback data to adjust internal parameters and templates. In one embodiment, the server trains or fine-tunes a model that predicts satisfaction from inputs such as emotional parameters, ingredient choices, and prompt sentence variants. The server then uses this model's predictions as part of the cost function for future decisions, thereby improving the likelihood of generating menus and recipes that lead to higher satisfaction. The server also updates weights in the ingredient selection optimization and may adjust boundaries in nutritional constraints for specific users, subject to safe limits.

The server can also update the prompt templates by statistically analyzing which prompt variants correlate with high satisfaction under similar health and emotional conditions. For example, the server can learn that adding explicit constraints on dish complexity or cleanup burden in the prompt sentence yields better outcomes for certain user profiles. The server then modifies the prompt template to include these constraints by default when conditions match those profiles. Because this adaptation occurs at the level of prompt construction and numeric parameter setting, it represents an improvement in the internal functioning of the computer-implemented system, rather than merely automating human menu planning.

By structuring all intermediate results as machine-readable data structures (nutrition target vectors, emotional parameter vectors, prompt templates, menu candidate graphs, ingredient lists with score annotations, and control command sequences), the server can use efficient index-based lookups, vectorized arithmetic, and caching. This leads to reduced computation time per planning cycle, and reduced network load to external generative AI services due to fewer unsuccessful or poorly constrained calls. The server also reduces communication load with the cooking apparatus by sending parameterized sequences rather than frequent low-level commands, while still allowing closed-loop corrections only when sensor readings indicate a need.

In alternative embodiments, the server may use different neural network architectures for the emotion estimation module, such as graph neural networks for multimodal fusion or variational autoencoders for robust feature extraction, or may use different optimization algorithms for ingredient selection, such as integer linear programming or heuristic search. The server may also integrate different types of generative AI models, including models specialized in recipe generation, while maintaining the core technique of programmatically constructing and iteratively refining prompt sentences based on structured state variables.

The described configuration results in a technical effect in that the server improves the functioning of the computing system by orchestrating emotion estimation, nutritional computation, prompt-based generative AI invocation, optimization-based ingredient selection, and feedback-based adaptation within a unified control framework. This configuration enables more accurate and efficient generation of menus and recipes that satisfy complex constraints, reduces processing errors in cooking apparatus control through sensor-based feedback, and improves resource utilization by minimizing unnecessary computation and communication.

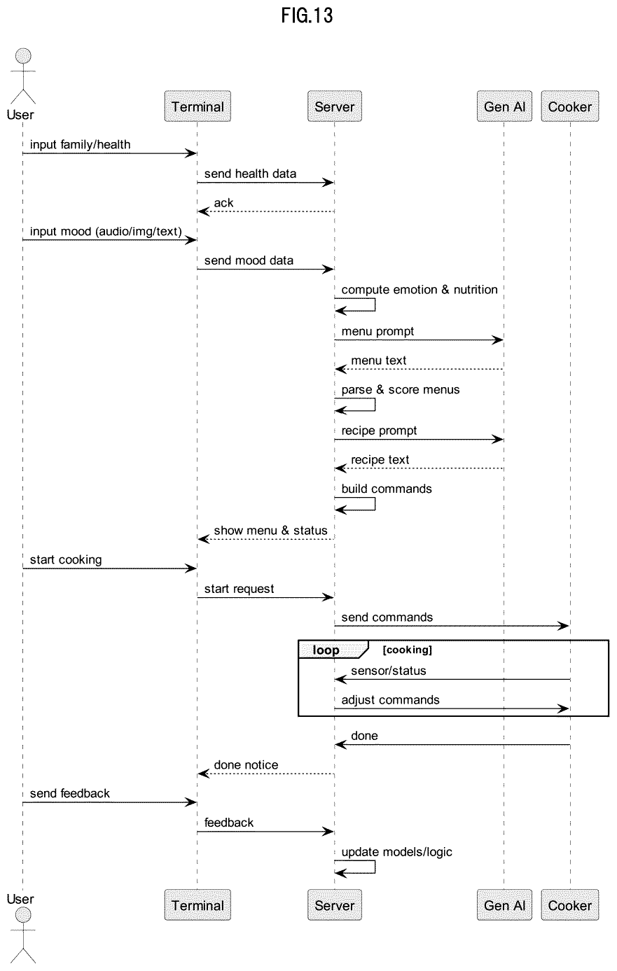

The following describes the processing flow using FIG. 13.

### Step 1

User operates the terminal to input family and health information.

User views an input screen on the terminal and enters, for each family member, age, sex, height, weight, disease information, allergy information, food preferences, and special nutritional needs using a keyboard, touch panel, or pointing device.

The input is raw keystrokes and touch events corresponding to textual and numeric fields.

Terminal receives these UI events as input, performs client-side validation such as required-field checks, numeric range checks (e.g., age must be between 0 and 120), and list-membership checks (e.g., allergy type must be one of predefined codes), and normalizes date and numeric formats.

Terminal outputs a structured data object, such as a hierarchical in-memory structure representing the family and health state data, and prepares it for transmission.

### Step 2

Terminal transmits health state data to the server.

Terminal takes the structured health state data as input, serializes it into a text-based format (for example, JSON) and encapsulates it into an HTTPS POST request with headers including authentication tokens and content type.

Terminal sends the request over a network interface to a predefined API endpoint on the server.

Terminal outputs an HTTP request carrying the serialized health state data.

### Step 3

Server receives and validates health state data.

Server takes the HTTP request body as input and uses a web framework to parse the serialized data into an internal data structure with fields mapped to application-level entities.

Server performs server-side validation, including checking that all required fields are present, that numeric values lie within allowed ranges, and that inter-field constraints hold (for example, body mass index not exceeding a sanity threshold).

Server then performs data cleaning such as trimming whitespace, normalizing text encodings, and converting categorical strings to internal codes.

Server outputs cleaned and validated records and, using a database API, stores them in tables such as a family information table and a health status table, or generates an error response if validation fails.

### Step 4

User provides emotional information through multimodal input.

User, using the terminal, selects a “Today's condition” or similar function and records a short voice message, captures a facial image or short video, and optionally enters free text describing mood and fatigue.

The input is raw audio samples, image frames from the camera sensor, and text characters entered via a keyboard.

Terminal converts the audio samples to a digital audio file, encodes the image frames into image files, and stores the text string in memory.

Terminal outputs a multimodal payload that includes the audio file, image file, and text string, and prepares it for transmission to the server.

### Step 5

Terminal sends multimodal emotion input to the server.

Terminal takes the multimodal payload as input, performs optional compression or encoding (e.g., base64 encoding for binary data), and constructs an HTTPS POST request targeting an emotion-input endpoint on the server.

Terminal includes metadata such as timestamps and user identifiers in the request body or headers.

Terminal outputs the request and transmits it via its network interface to the server.

### Step 6

Server performs multimodal emotion feature extraction.

Server receives the multimodal payload as input and separates it into audio data, image data, and text data.

Server uses a digital signal processing library to transform the audio waveform into a feature matrix, computing features such as Mel-frequency cepstral coefficients, short-term energy, and pitch.

Server uses an image processing library to detect facial regions in the image, crops and resizes the detected face region, and converts the pixel data into a normalized tensor.

Server uses a text processing library to tokenize the text into subword units or word tokens and converts these tokens into integer indices or embeddings.

Server outputs three sets of feature representations: an audio feature tensor, an image feature tensor, and a text feature tensor.

### Step 7

Server estimates emotional parameters using machine learning models.

Server takes the audio, image, and text feature tensors as input and feeds them into corresponding trained neural network models: for example, a recurrent or transformer-based network for audio, a convolutional network for images, and a transformer-based language model for text.

Server performs forward propagation through these models, applying matrix multiplications, non-linear activation functions, and normalization layers.

Server obtains probability distributions over emotion classes for each modality, such as probabilities for “high stress,” “low stress,” “high fatigue,” and “positive mood.”

Server then applies a multimodal fusion algorithm, such as a small fully connected network or a weighted average function, using the modality-specific outputs as input.

Server outputs normalized emotional parameters, including at least a numeric stress level, a numeric fatigue level, and optionally a positivity score and event mood classification, and stores these parameters in an emotion information table.

### Step 8

Server calculates nutritional targets for each family member.

Server reads health state records from the health status table as input, including age, sex, disease codes, and special nutritional needs.

Server queries a nutrition reference table containing recommended nutrient intakes and applies a selection algorithm to retrieve reference rows corresponding to each member's age and sex.

Server then applies rule-based adjustments depending on disease codes (e.g., lowering allowable carbohydrates for diabetes, lowering sodium for hypertension) and computes per-day and per-meal required nutrient amounts via arithmetic operations such as multiplication and division.

Server aggregates the results into a structured vector of required nutrient amounts per member and per meal.

Server outputs these vectors as nutritional targets and stores them in a nutrition target table.

### Step 9

Server generates a prompt sentence for menu planning using the generative AI model.

Server takes as input the nutritional targets, emotional parameters, family composition information, preference information, season information, and event information from the respective tables.

Server uses a template-based text generator to insert these parameters into a predefined natural language structure, converting numeric nutrient values and emotional scores into human-readable constraints (e.g., “limit salt,” “keep cooking time within 30 minutes,” “user is very tired”).

Server concatenates these segments into a single prompt sentence that specifies conditions for meals, including nutritional goals, emotional constraints, and contextual conditions.

Server outputs a complete prompt sentence such as:

“Based on the following conditions, propose 3 dinner menu options for today.

### [Family Composition and Health Status]

. . .

### [Current User Emotional State]

The user is very tired from work and has a high stress level.
Total cooking time should be within 30 minutes, with as few steps as possible and minimal dishes to wash.

### [Other Conditions]

The current season is winter, and at least one dish should be warming for the body.

Please output 3 menu sets (Japanese or Western style) that satisfy all the above conditions, in Japanese.”

### Step 10

Server queries the generative AI model for menu candidates.

Server takes the prompt sentence as input and builds a request payload for a generative AI model API, including the prompt sentence, model identifier, temperature, maximum token count, and language parameters.

Server sends the request over HTTPS to an external generative AI model service and waits for a response.

The generative AI model service returns a text response containing multiple menu descriptions.

Server receives the response, extracts the textual content, and outputs the raw menu description text.

### Step 11

Server parses the generative AI output into structured menu candidates.

Server takes the raw menu description text as input and applies parsing logic that uses line-based splitting, pattern matching, and regular expressions to identify separate menu candidates and individual dishes.

Server detects markers such as numbered lists or labeled sections (“Main dish,” “Side dish,” etc.) and maps them to internal data structures.

Server constructs a menu candidate object for each suggested menu, with fields representing dish type, dish name, and simple description.

Server outputs an array or collection of structured menu candidates and stores them in a menu candidate table.

### Step 12

Server links dishes to standard recipes and computes nutrient totals.

Server takes the structured menu candidates as input and, for each dish, queries a recipe information table to obtain standard recipes that specify ingredient types and quantities per serving.

Server uses these recipes to build ingredient sets for each menu, multiplying quantities by the number of servings corresponding to family size.

Server then queries a food composition table to obtain nutrient content per unit weight for each ingredient, and performs vectorized multiplications and summations to compute total nutrient values per menu.

Server outputs nutritional profiles for each menu candidate, including total energy, macronutrients, and key micronutrients.

### Step 13

Server evaluates menu candidates against nutritional targets and emotional constraints.

Server takes the nutritional profiles and the per-meal required nutrient amounts as input and compares each nutrient dimension, computing absolute or relative deviations from target ranges.

Server also reads emotional parameters and applies scoring rules that penalize menus with long preparation times, many steps, or complex cooking methods when fatigue and stress levels are high.

Server calculates a composite score for each menu candidate based on nutritional compliance and emotional suitability.

Server outputs a ranked list of menu candidates and, if all candidates fail nutritional thresholds, modifies the constraints in the prompt sentence and triggers a new call to the generative AI model.

### Step 14

Server generates an intermediate ingredient list from the selected menu.

Server takes the highest-ranked menu candidate as input and aggregates all ingredients required for its dishes by summing quantities of identical ingredients across dishes.

Server constructs an intermediate ingredient list where each entry includes an ingredient identifier and a total required quantity.

Server outputs this intermediate ingredient list and stores it in an ingredient planning table.

### Step 15

Server selects ingredient forms and vendors based on emotional parameters and external product data.

Server takes the intermediate ingredient list and emotional parameters as input and queries an ingredient catalog table for available forms (e.g., fresh, pre-cut, marinated, frozen) for each ingredient.

Server then calls external product provision APIs to obtain price and stock information for each form at various vendors.

Server applies an optimization algorithm that computes a score for each candidate form and vendor combination using a cost function that weights monetary cost and user preparation burden, with the burden weight increased when fatigue level is high.

Server selects the combination with the lowest weighted score for each ingredient and compiles a final ingredient list that specifies product IDs, forms, quantities, and vendors.

Server outputs this final ingredient list and records it in an ingredient list table.

### Step 16

Server schedules delivery and places orders with external services.

Server takes the final ingredient list and user schedule information as input and computes feasible delivery windows by intersecting vendor delivery slots with user at-home intervals.

Server may also partition the ingredient list into same-day and later-day groups based on emotional parameters and complexity of planned menus.

Server constructs order messages that include product identifiers, quantities, and selected delivery windows and sends them to ordering and delivery service devices via their APIs.

Server receives confirmation messages including order IDs and scheduled times, and updates the order status table.

Server outputs updated order status information for subsequent display on the terminal.

### Step 17

Server generates a recipe-generation prompt sentence for the selected dish.

Server takes the selected menu, cooking apparatus specification information, and emotional parameters as input and constructs a recipe-generation prompt sentence.

Server inserts constraints such as maximum number of user operations, explicit numeric temperature and time values per step, and the requirement to separate “User operation” and “Robot automatic operation.”

Server also specifies allergy constraints for family members and texture constraints for elders.

Server outputs a detailed recipe-generation prompt sentence like:

“For the dish ‘Chicken and Root Vegetable Soy Milk Style Soup (dairy-free),’ generate a recipe suitable for cooking with a home automatic cooking robot.

### Conditions:

The user is very tired from work, so user operations must be minimized. User operations should be limited to washing ingredients, roughly cutting them, and putting them into the robot pot.
The robot must be able to automatically control heating temperature, heating time, and stirring timings, so specify numerical values for temperature (C) and time (minutes) for each step.
The child has a dairy allergy, so do not use any dairy products such as milk or cheese. Use alternatives such as soy milk.
The grandmother is elderly, so ingredients should be cooked until soft and easy to eat.

Write the procedure with numbered steps, and for each step clearly separate ‘User operation’ and ‘Robot automatic operation’ in the description.”

### Step 18

Server queries the generative AI model for a detailed recipe.

Server takes the recipe-generation prompt sentence as input and sends it to the generative AI model API with appropriate generation parameters.

Server receives a response containing a multi-step recipe text with numbered steps and textual descriptions.

Server extracts the recipe text and outputs it as unstructured text for further parsing.

### Step 19

Server parses the recipe text into a structured step representation.

Server takes the recipe text as input and applies text parsing functions to identify step numbers, user actions, and robot actions.

Server locates numeric expressions corresponding to temperatures and times and parses them into numeric fields, and identifies stirring or mixing instructions and their timing.

Server constructs a sequence of step records, each including fields such as step ID, user operation description, robot mode, target temperature, duration, and stirring intervals.

Server outputs this structured step representation and stores it in the cooking command table as an intermediate form.

### Step 20

Server converts structured steps into a control command sequence for the cooking apparatus.

Server takes the structured step representation and cooking apparatus specification information as input and maps each robot mode and numeric parameter to one or more device-specific command messages according to the apparatus control protocol.

Server ensures that commands respect capacity and temperature limits defined in the specification information and orders them according to step dependencies.

Server constructs an ordered control command sequence, where each command includes operation type, parameters, and timing.

Server outputs this control command sequence, ready to be sent to the cooking apparatus.

### Step 21

Terminal presents menus, ingredient information, and status to the user.

Terminal receives from the server the structured menu candidates, the final ingredient list, and order status information as input.

Terminal transforms this data into user interface elements such as menu lists, ingredient summaries, and delivery time displays.

Terminal renders these elements on the display, allowing the user to inspect options and make selections.

Terminal outputs user interaction events when the user selects a menu and presses a “Start cooking” button.

### Step 22

Terminal sends a cooking start instruction to the server.

Terminal takes the user's selection and action as input and constructs a control instruction message including the chosen menu ID and optional timing preferences.

Terminal sends this instruction to the server via HTTPS as a request targeting a cooking-start endpoint.

Terminal outputs the request and may display a waiting or progress screen until the server acknowledges reception.

### Step 23

Server initiates cooking by sending control commands to the cooking apparatus.

Server receives the cooking start instruction as input, retrieves the corresponding control command sequence from the cooking command table, and packages it into a message payload according to the cooking apparatus communication protocol.

Server transmits this sequence to the cooking apparatus over the network interface, optionally including metadata like total estimated cooking time.

Server outputs an initiation acknowledgement to the terminal and starts monitoring for feedback from the cooking apparatus.

### Step 24

Server performs closed-loop monitoring and adjustment of cooking.

Server receives temperature sensor information, timer information, and optionally image information from the cooking apparatus as input streams.

Server compares measured temperatures against target temperatures and evaluates time deviations for each step; optionally, server analyzes images to estimate doneness or browning.

Server applies control logic that, on detecting insufficient temperature or undercooking, computes corrections such as extending heating time or slightly increasing target temperature within safe limits.

Server then generates additional control commands reflecting these corrections and transmits them to the cooking apparatus.

Server outputs updated control sequences and status data, ensuring that cooking adapts to real

### Step 25

Server notifies the terminal and records feedback after cooking completion.

Server receives a completion status from the cooking apparatus as input and updates internal records for the cooking session.

Server then constructs a completion notification message, potentially including a message tailored to emotional parameters (e.g., indicating that the dish is kept warm).

Server sends this notification to the terminal, and the terminal displays it to the user.

User then provides satisfaction evaluation information via the terminal UI.

Terminal sends the feedback data to the server, and server stores this feedback in association with the menu, emotional parameters, and decision history, outputting updated learning data that will be used to refine future nutrition calculations, prompt sentence templates, and ingredient selection weights.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional menu planning and cooking support systems typically separate nutritional computation, shopping support, and cooking device control into loosely coupled modules that operate with static rules. In such systems, nutritional requirements are calculated once based on static health profiles, ingredient lists are generated by simple aggregation, and delivery schedules are computed by fixed heuristics or manual configuration. Furthermore, cooking device control is often performed by pre-defined recipes that do not adapt to real-time user state. As a result, when these systems are applied to care receivers and caregivers, they fail to react dynamically to changes in emotional states, fatigue, or long-term behavioral patterns.

From the viewpoint of computer technology, this separation creates several technical limitations.

First, the system cannot efficiently integrate heterogeneous data types—such as structured health data, time-series sensor data from a cooking apparatus, unstructured natural language, and multimodal emotion signals (image, audio, text)—into a unified decision pipeline. This leads to redundant processing, repeated manual adjustments, and increased latency and resource usage, because each module independently processes partial information without sharing learned context.

Second, existing systems are not designed to use generative AI models in a systematic and programmable way. Prompt sentences, if used at all, are typically static or manually crafted, and are not generated and updated programmatically based on machine-readable profiles and emotion vectors. As a result, the outputs of generative AI models cannot be stably parsed into structured data and cannot be reliably fed into downstream algorithms for ingredient planning, delivery scheduling, and device command generation. This limits the ability of the system to scale, to be automated, and to maintain robustness in the face of noisy or changing inputs.

Third, conventional architectures do not implement a closed feedback loop from post-meal outcomes-such as satisfaction scores and measured emotion changes-back into the computational logic that constructs prompts and interprets AI outputs. Without such a loop, the system cannot learn which patterns of menus, delivery plans, and cooking procedures are technically effective in improving engagement and reducing intervention needs. This prevents the system from adapting its internal models and prompt-generation strategies over time, and leads to suboptimal utilization of computing resources, repeated generation of low-acceptance outputs, and unnecessary re-execution of planning and control processes.

Fourth, existing systems generally treat the cooking device as a passive endpoint that executes fixed commands, with limited monitoring and adjustment. There is no integrated mechanism to programmatically synthesize, from AI-generated recipes, low-level control command sequences that are parametrized by emotion vectors and that are dynamically corrected based on sensor feedback. As a consequence, the system cannot optimize device operation in terms of stability, noise, latency, or fault recovery, and cannot systematically reduce the number of manual interventions needed from the caregiver.

Therefore, there is a need for an improved computer-implemented system and method that (i) programmatically generates and updates prompt sentences for a generative AI model based on structured health profiles and multi-source emotion vectors, (ii) parses AI outputs into well-defined intermediate data structures that feed directly into ingredient planning, delivery scheduling, and device control pipelines, (iii) incorporates a feedback mechanism using post-meal evaluation data and emotion changes to refine these prompts and internal parameters over time, and (iv) integrates real-time monitoring and dynamic correction of cooking device commands. Such a system should improve computational efficiency, robustness, and adaptability of the entire pipeline, thereby enhancing the overall performance of the underlying computer technology for personalized meal planning and cooking automation.

The specific processing by the specific processing unit 290 of the data processing device 12 in Application Example 2 is realized by the following means.

The present invention provides a server comprising a processor and a memory storing instructions that, when executed by the processor, cause the server to implement an integrated data processing pipeline that unifies health information management, emotion state estimation, prompt sentence generation for a generative AI model, structured parsing of AI outputs, planning of ingredient procurement and delivery, and generation and dynamic correction of control commands for a cooking apparatus. This enables the server to improve computational efficiency and robustness by automatically transforming heterogeneous inputs into machine-readable intermediate data, by programmatically controlling interactions with the generative AI model through dynamically configured prompt sentences, and by closing a feedback loop in which post-meal evaluation data and emotion changes are used to adapt future prompts, planning logic, and device control parameters.

The present invention provides a server comprising a processor configured to receive, via an input interface, health information and preference information for a care receiver and a caregiver, to normalize and store this information as structured health data in a storage device, and to calculate, based on the stored health data, nutritional profile data that includes target energy amounts, target macronutrient ratios, micronutrient targets, and ingredient constraint information. This enables the server to construct machine-readable nutritional requirements that are directly consumable by a later stage of the pipeline, rather than relying on ad hoc or manual nutritional specifications.

The present invention further provides a server comprising a processor configured to acquire, via one or more client terminals, multimodal emotion-related inputs including image data, audio data, and text data, to perform facial expression analysis, audio emotion analysis, and text emotion analysis to generate respective emotion scores, and to integrate the emotion scores into an emotion vector data structure whose components represent multiple emotion indices such as joy, anxiety, stress, fatigue, and motivation. This enables the server to maintain a unified, numeric representation of emotional state, which can be programmatically applied across multiple computational modules without the need for repeated feature extraction or ad hoc interpretation.

The present invention further provides a server comprising a processor configured to generate, based on the nutritional profile data and the emotion vector data, a first prompt sentence for a generative AI model, the first prompt sentence describing nutritional constraints, prohibited ingredients, and emotion states of the care receiver and the caregiver, to transmit the first prompt sentence to the generative AI model, to receive a menu proposal text from the generative AI model, and to parse the menu proposal text into structured menu data including, for each meal category, dish identifiers, ingredient lists, and nutritional attributes. This enables the server to use the generative AI model as a deterministic computational component within a larger system, by controlling the model via machine-generated prompts and immediately converting the natural language outputs back into canonical data structures used by the program.

The present invention further provides a server comprising a processor configured to compute, based on the menu data, ingredient list data by aggregating ingredient types and required amounts, to obtain, from external product information services, candidate product data including stock status, price, processing form, and delivery options, and to generate, based on the ingredient list data, the caregiver's emotion vector data, and planned ingredient usage dates, a second prompt sentence describing constraints on delivery dates and times, number of deliveries, and processing forms of ingredients. The processor is further configured to transmit the second prompt sentence to the generative AI model, receive a delivery schedule proposal text, parse the delivery schedule proposal text into delivery schedule data specifying dates, time windows, and ingredient groups, and send order requests to external order and delivery services based on the delivery schedule data. This enables the server to offload complex, constraint-rich scheduling logic to the generative AI model in a controlled, auditable manner, while guaranteeing that the resulting schedule is re-expressed in a form that downstream ordering and logistics components can consume automatically.

The present invention further provides a server comprising a processor configured to generate, based on the menu data, the nutritional profile data, the emotion vector data, and function information of a cooking apparatus, a third prompt sentence that describes cooking conditions including cooking modes, heating temperatures, heating times, stirring conditions, and required manual operations, to transmit the third prompt sentence to the generative AI model, and to receive and parse a recipe text from the generative AI model into cooking procedure data in which each step is represented as a machine-readable structure. This enables the server to transform high-level, text-based recipe descriptions generated by the generative AI model into explicit procedural representations suitable for direct mapping into low-level control commands.

The present invention further provides a server comprising a processor configured to translate the cooking procedure data and the emotion vector data into a sequence of control commands for a cooking apparatus, the control commands including cooking mode setting commands, temperature setting commands, timer setting commands, and stirring control commands, and to adjust operation speed, operation sound, and notification frequency of the cooking apparatus in accordance with the emotion states of the care receiver and the caregiver. The processor is further configured to transmit the sequence of control commands to the cooking apparatus, to receive sensor data and operation state data from the cooking apparatus, to compare the data with predetermined thresholds, and to dynamically modify the sequence of control commands to correct deviations, including extending heating times or adjusting temperatures. This enables the server to implement a closed-loop device control mechanism that automatically adapts operation parameters to emotional and physical states and that reacts to runtime deviations, thereby improving reliability and reducing the need for manual intervention.

The present invention further provides a server comprising a processor configured to obtain, after provision of a meal, satisfaction evaluation data from the care receiver or caregiver and post-meal emotion vector data, to compute changes in emotion indices by comparing pre-meal and post-meal emotion vectors, and to analyze correlations between menu patterns, cooking procedures, and emotion changes over time. The processor is configured to update internal models and to incorporate analysis results as additional conditions when generating the first, second, and third prompt sentences for subsequent interactions with the generative AI model. This enables the server to implement a learning mechanism inside the prompt-generation and planning logic so that, over time, the system converges toward menu patterns, delivery strategies, and device control procedures that measurably improve emotional outcomes and reduce computational waste associated with generating low-acceptance outputs.

By configuring the processor to perform these operations in an integrated and automated manner, the present invention improves the functioning of the underlying computer system itself. The system reduces redundant processing across modules by sharing standardized intermediate structures such as nutritional profiles, emotion vectors, and cooking procedures; makes efficient and predictable use of generative AI models via programmatically generated prompt sentences; and provides a closed feedback loop that updates these prompts and control parameters based on observed outcomes. As a result, the computer system achieves improved scalability, robustness, and adaptability in processing multimodal data and controlling external devices in the specific context of personalized meal planning and cooking automation.

The term “health information” refers to structured or unstructured data representing physical or medical attributes of a person, including at least age, sex, body size, existing diseases, medication information, allergy information, swallowing function information, and other clinically relevant conditions.

The term “preference information” refers to data indicating individual likes, dislikes, and constraints regarding food or meals, including at least favored cuisines, disliked ingredients, texture preferences, and cultural or religious dietary preferences.

The term “care receiver” refers to a person for whom meals, nutrition, and cooking support are provided by the system, and whose health information, preference information, and emotion information are managed by the server.

The term “caregiver” refers to a person who supports the care receiver in daily life, including preparation and supervision of meals, and whose emotion information and workload state may be considered by the system.

The term “health data” refers to normalized, structured data derived from health information and preference information, stored in a storage device in association with an identifier of the care receiver or caregiver.

The term “nutritional requirement amount” refers to a computed target quantity for dietary energy and nutrients for a given period, determined based on health data and nutritional standards, including at least a target energy amount and target macronutrient and micronutrient amounts.

The term “nutritional constraint condition” refers to restrictions or bounds on dietary intake derived from health data, such as upper or lower limits on energy, macronutrient ratios, sodium, or specific nutrients, and prohibitions or limitations relating to particular ingredients.

The term “nutritional profile data” refers to a machine-readable data structure that represents nutritional requirement amounts and nutritional constraint conditions for a given person, including at least target energy, macronutrient ratios, micronutrient targets, and ingredient constraint information.

The term “prohibited ingredient list” refers to a set of ingredient identifiers that are to be avoided for a given person based on allergies, medical conditions, or other constraints, and that are represented as elements of nutritional profile data.

The term “image information” refers to digital image data, including still images or frames extracted from video, that depict at least a face or body of a care receiver or caregiver and that are used for expression or emotion analysis.

The term “audio information” refers to digital audio data, including voice recordings of the care receiver or caregiver, that are used for emotion analysis and automatic speech recognition.

The term “text information” refers to digital textual data obtained from user input, recognized speech, or other sources, and used for emotion analysis and interpretation of content.

The term “expression analysis” refers to processing that detects a face region in image information and classifies facial expressions into categories, and that generates scores related to emotions based on facial characteristics.

The term “audio emotion analysis” refers to processing that extracts acoustic features from audio information and classifies the acoustic features into emotion categories or scores that represent emotional states.

The term “text emotion analysis” refers to processing that analyzes text information using a language model or classifier to estimate sentiment or emotional categories or scores associated with the textual content.

The term “emotion indices” refers to numerical factors that represent different aspects of an emotional state, including at least joy, anxiety, stress, fatigue, and motivation, each expressed on a defined numeric scale.

The term “emotion vector data” refers to a multi-dimensional numeric data structure in which each dimension corresponds to an emotion index and stores a numerical value representing the strength of that emotion at a particular time.

The term “emotion state” refers to a psychological or affective condition of the care receiver or caregiver at a given time, expressed by at least one emotion vector or a set of emotion indices.

The term “generative AI model” refers to a machine-learned model that generates new text or other content in response to input data, including at least a large language model which generates natural-language outputs based on prompt sentences supplied by the server.

The term “prompt sentence” refers to a text sequence transmitted to a generative AI model, the text sequence specifying a task, constraints, or conditions and requesting a particular type or format of output from the generative AI model.

The term “menu proposal text” refers to a natural-language output generated by the generative AI model in response to a prompt sentence relating to menu generation, the output including at least dish descriptions and nutritional indications.

The term “menu data” refers to structured data obtained by parsing menu proposal text, including for each meal category at least dish identifiers, dish names, associated ingredient lists, and nutritional attributes.

The term “meal category” refers to a classification of a meal within a time-based or functional division such as breakfast, lunch, dinner, or snack.

The term “ingredient list data” refers to structured data representing a set of ingredients and corresponding required amounts or purchase units for implementation of one or more menus.

The term “planned use date” refers to a date or time period assigned to a menu or ingredient, indicating when the corresponding ingredient is expected to be used in meal preparation.

The term “delivery schedule proposal text” refers to a natural-language output generated by the generative AI model in response to a prompt sentence relating to delivery scheduling, the output including proposed delivery dates, time windows, and groupings of ingredients.

The term “delivery schedule data” refers to structured data derived from a delivery schedule proposal text, specifying, for each delivery event, at least a date, a time window, and a set of ingredients or products to be delivered.

The term “external order and delivery service” refers to a remote computer-implemented service or system that receives order requests for goods and arranges delivery of the goods according to specified dates, times, and locations.

The term “function information of a cooking apparatus” refers to capability data of a cooking device, including at least supported cooking modes, controllable temperature range, controllable heating durations, stirring capabilities, and other operational parameters.

The term “cooking conditions” refers to a set of parameters specifying how a dish is to be cooked by a cooking apparatus, including at least cooking modes, heating temperatures, heating times, stirring conditions, and indications of required manual operations.

The term “recipe text” refers to a natural-language output generated by the generative AI model in response to a prompt sentence relating to recipe generation, the output describing ingredients and step-by-step cooking instructions.

The term “cooking procedure data” refers to structured data obtained by parsing recipe text, the structured data representing cooking steps with parameters such as step order, mode, temperature, time, stirring condition, and ingredient addition timing.

The term “control command” refers to a machine-readable instruction transmitted to a cooking apparatus, specifying at least one operation such as setting a cooking mode, setting a temperature, controlling a timer, or controlling a stirring mechanism.

The term “sequence of control commands” refers to an ordered collection of control commands that, when executed by a cooking apparatus, cause the cooking apparatus to carry out an entire cooking procedure automatically or semi-automatically.

The term “operation speed” refers to a parameter indicating how quickly mechanical components of a cooking apparatus, such as motors or stirrers, operate during execution of a cooking procedure.

The term “operation sound” refers to acoustic characteristics produced by a cooking apparatus during operation, which can be influenced by operation modes, speeds, or other control parameters.

The term “notification frequency” refers to a rate at which the system sends status messages, alerts, or guidance notifications to a user through a terminal or other interface during planning or cooking.

The term “temperature data” refers to sensor readings or measurement values transmitted from a cooking apparatus, indicating current temperatures of one or more parts of the apparatus or of the food being cooked.

The term “operation state data” refers to status information transmitted from a cooking apparatus, including at least indications of current mode, timer values, motor states, and error conditions.

The term “satisfaction evaluation data” refers to feedback data obtained from a care receiver or caregiver after a meal, including numeric or categorical ratings of taste, ease of eating, overall satisfaction, or perceived mood changes.

The term “post-meal emotion vector data” refers to emotion vector data collected or computed after a meal, representing the emotional state of a care receiver or caregiver following consumption of the meal.

The term “menu patterns” refers to recurrent or categorized combinations of dishes, ingredients, or nutritional profiles, identified across multiple menus over time.

The term “emotion changes” refers to differences between emotion indices before and after a given event, such as a meal, derived by comparison of emotion vectors.

The term “long-term preference tendencies” refers to trends or patterns in accepted menus, ingredients, or cooking styles over a prolonged period for a particular care receiver, inferred from historical menu data and feedback.

The term “long-term emotion tendencies” refers to trends or patterns in emotion indices of a care receiver or caregiver across multiple events, indicating stable or slowly changing emotional profiles over time.

The term “client terminal” refers to an information processing device operated by a user, such as a smartphone, tablet, or personal computer, configured to exchange data with the server, present user interfaces, and capture image, audio, or text input.

The term “server” refers to an information processing system comprising at least one processor and at least one memory, configured to execute the data processing, AI communication, planning, and device control steps described in the claims.

In one embodiment, a system includes a server, at least one terminal, and at least one cooking apparatus interconnected via a communication network. The server comprises at least one processor and at least one memory storing machine-executable instructions. The terminal comprises an information processing device such as a smartphone, tablet computer, or personal computer. The cooking apparatus comprises a controllable cooking device such as a smart cooker or multi-function cooking robot including a heater, a motor, one or more sensors, and a network interface.

The server executes an operating system such as a general-purpose UNIX-like operating system and runs an application stack including a web application framework, an application server, and a database management system. In one configuration, the server executes an HTTP-based application framework implemented in a high-level language, an application server process for handling concurrent requests, and a relational database system such as a relational database management engine for persistence of structured data. The server additionally executes one or more machine learning inference engines, including a convolutional neural network for facial expression recognition, a neural network for audio emotion recognition, a transformer-based language model for text emotion analysis, and client libraries for accessing an external generative AI model via an application programming interface.

The terminal executes a dedicated care-support application on a mobile or desktop operating system.

The terminal comprises a camera, a microphone, a touch-sensitive display, and optionally a speaker.

The terminal uses these components to capture user inputs, including health information, preference information, images of the care receiver's face, recordings of the care receiver's or caregiver's voice, and free-form textual comments. The terminal converts such inputs into structured formats such as JSON, image formats such as JPEG, and audio formats such as WAV, and transmits them to the server via secure network protocols.

The user operates the terminal to input health information and preference information. The user enters, for example, age, sex, body height, body weight, existing diseases, current medications, allergy information, swallowing function constraints, and food preferences through graphical user interface elements such as forms, dropdown lists, and sliders. The terminal packages these attributes into key-value pairs and sends them to the server.

The server receives the information and performs normalization and validation of the health data. The server uses schema validation libraries to check data types, ranges, and required fields. The server converts free-form attributes into canonical codings using dictionaries or mapping tables (for example mapping a disease name to a standardized disease code). The server stores the normalized health data in multiple relational tables, such as a patient table, a medical condition table, and a preference table. Each record is associated with a patient identifier and timestamp, enabling the server to track updates and compute longitudinal profiles.

The server computes nutritional profile data based on the stored health data. The server loads nutritional standard tables into an in-memory data structure such as a two-dimensional table indexed by age, sex, and activity level. The server applies predetermined formulas for basal metabolic rate and total energy expenditure, and then calculates target macronutrient ratios and micronutrient targets. For example, the server calculates a daily target energy amount in kilocalories, a carbohydrate ratio in percent, a protein ratio in percent, and a fat ratio in percent. The server further reads disease information and applies rule-based constraints, such as limiting carbohydrate ratio for diabetes or restricting sodium for cardiovascular conditions. These rules are stored as condition-action mappings and not as simple human-readable guidelines, allowing the server to apply them programmatically and consistently.

The server generates nutritional profile data as a structured object that includes fields such as target energy, target macronutrient ratios, minimum or maximum nutrient amounts, and a prohibited ingredient list derived from allergy and medical condition data. The server stores this nutritional profile data into a dedicated profile table, linked to the patient identifier. By using this structured representation, the server can reuse the same nutritional profile across multiple computational modules without recalculating fundamental nutritional targets, which reduces redundant computation and improves overall system throughput.

The terminal acquires emotion-related data by guiding the user to perform a short “condition check.” The user positions the terminal so that the care receiver's face is centered in the camera view and initiates capture. The terminal captures a short sequence of video frames and audio. The terminal may subsample frames to a fixed rate and compress them in a lossless or lossy image format optimized for downstream analysis. The terminal also records audio at a predetermined sampling rate such as 16 kHz to ensure compatibility with pre-trained audio models. The terminal transmits the media data to the server using multipart requests carrying both binary data and metadata like patient identifiers and capture timestamps.

The server stores the uploaded images and audio into a media file store and records their paths and metadata in a media index table. The server then performs multimodal emotion analysis.

For facial expression analysis, the server uses a convolutional neural network. In one implementation, the server employs a convolutional backbone with residual connections, batch normalization layers, and a softmax output layer producing probabilities over a fixed set of expression categories. The server first uses a face detection model—such as a multi-layer convolutional detector trained on annotated face bounding boxes—to identify face regions within each captured image. The server then crops and resizes these regions and normalizes pixel values. The server feeds the processed face images into the expression classifier and obtains probability vectors for expressions such as joy, anger, sadness, surprise, and neutral. The server aggregates the per-frame outputs by computing a weighted average or other statistical measure, thereby reducing noise due to frame variations.

For audio emotion analysis, the server extracts acoustic features from the audio waveform. The server computes either mel-spectrograms or filterbank features and arranges them into sequences in time.

The server inputs these sequences into an audio emotion model such as a neural network having convolutional time-domain layers followed by recurrent or transformer layers and a classification head that outputs probabilities for emotions such as joy, anxiety, anger, and calm. Such a model has been previously trained on labeled emotion datasets using supervised learning, with an objective function such as cross-entropy loss and an optimizer such as stochastic gradient descent or Adam, and optionally data augmentation techniques such as random time shifting or noise injection to improve robustness.

For text emotion analysis, the server performs automatic speech recognition. The server invokes a speech recognition engine that implements an encoder-decoder or transformer-based architecture.

The engine converts audio sequences into sequences of text tokens. The server then inputs the recognized text into a language model-based classifier, for example a transformer model pre-trained on large corpora and fine-tuned on emotion classification tasks. This classifier outputs scores for categories such as positive, negative, anxiety, and stress. The server may normalize these scores or map them to a standard 0-10 scale.

The server combines the expression-based scores, audio-based scores, and text-based scores by applying a weighting scheme. The server assigns higher weight to the modality deemed more reliable under current conditions. For instance, when speech is clear and continuous, the server may give greater weight to text-based scores; when the audio is noisy but facial expressions are clearly visible, the server may emphasize visual emotion scores. The server performs a weighted sum and then normalizes each dimension to a predefined range, producing emotion vector data. The emotion vector comprises numeric values for indices such as joy, anxiety, stress, fatigue, and motivation.

The server stores the resulting emotion vector in an emotional state table, indexed by patient identifier, caregiver identifier (when applicable), and timestamp. This structure allows the server to track changes over time and supports later analysis of emotion transitions and correlations with menus and device operations.

The server interacts with a generative AI model using dynamically constructed prompt sentences.

The generative AI model may be a large language model that uses a transformer-based architecture with self-attention layers, having been pre-trained on a broad corpus of natural language and optionally fine-tuned on domain-specific tasks such as menu generation or recipe authoring. The server does not treat this model as a black box that arbitrarily “decides,” but rather as a deterministic function from text input to text output, constrained by prompt design and sampling parameters. The server sets parameters such as temperature, maximum token length, and top-k or nucleus sampling thresholds so that outputs are sufficiently constrained to allow reliable parsing.

The server generates a first prompt sentence to request a menu proposal from the generative AI model. The server reads the nutritional profile data and the current emotion vectors of the care receiver and the caregiver. The server embeds these values into a structured text template that clearly labels health conditions, target nutrients, prohibited ingredients, emotional states, and desired output formats. One example of such a prompt sentence is as follows:

“You are an expert in nutrition for the elderly and are highly skilled at creating home-style menus.

Please propose a one-day menu (breakfast, lunch, and dinner) in Japanese for the care receiver under the following conditions.

### [Health Conditions]

Age: 82 years, female
Diseases: Type 2 diabetes, osteoporosis risk
Allergy: All nuts
Target nutrients: Approx. 1600 kcal; carbohydrates 45-50%; protein 20-25%; fat 25-30%; with higher calcium and vitamin D

### [Emotional State]

The care receiver currently has high anxiety about meals (7/10) and low interest in new dishes (2/10), so focus on familiar Japanese home-style dishes that give a sense of security.
The caregiver has high fatigue (8/10), so prioritize menus where many steps can be handled by a cooking robot and that require minimal preparation work.

### [Output Format]

For breakfast, lunch, and dinner, output the dish names, short descriptions, and key ingredients as bullet points.
For each meal, also show approximate carbohydrate amount and calories.”

The server transmits this text via HTTP to a generative AI endpoint and receives the response text describing the menu. The server then performs deterministic parsing of the response. To support reliable parsing, the server encourages the model through the prompt to use explicit headings and bullet formats. The server identifies segments corresponding to breakfast, lunch, and dinner, then extracts dish names, descriptions, ingredient lists, and nutritional approximations. The server converts these into menu data records using pre-defined schemas and stores them into menu and dish tables.

This structured representation ensures that downstream planning and control modules can consume AI-generated content without direct human adjustment.

The server computes ingredient list data based on the menu data. The server consults a recipe master that maps each dish to its base ingredient list and standard quantities. The server multiplies these base quantities by the number of servings and aggregates the ingredient quantities across all dishes of the planning horizon. The server then rounds up the quantities to the nearest purchase units by referencing an ingredient master table containing conversion factors and packaging information.

The server queries external product information services. The server constructs HTTP requests with search terms corresponding to ingredient names and attributes and receives structured responses describing available products, including stock status, price, packaging, processing form (for example fresh, pre-cut, frozen), and delivery options. The server stores this product candidate information into a product candidate table.

The server constructs a second prompt sentence for delivery scheduling. The server summarizes ingredient needs by date, incorporates product processing forms, and reads the caregiver's emotion vector, particularly the fatigue index. The server also reads user-specified preferred delivery time windows. The server inserts all of these elements into a text template designed to elicit a schedule that balances freshness, number of deliveries, and burden reduction. One example of a second prompt sentence is:

“Based on the following conditions, create a one-week delivery schedule for ingredients.

### [Ingredient List]

Ingredients to be used on Tuesday: [ . . . ]
Ingredients to be used on Thursday: [ . . . ]

### [Caregiver State]

Fatigue level: 8/10
On weekdays the caregiver is busy during the day, and prefers to receive deliveries on weekday evenings (19:00-21:00) and Saturday mornings (9:00-11:00).

### [Conditions]

To reduce cooking burden, if pre-cut or pre-prepared ingredients are available, prioritize them.
For highly perishable ingredients (fresh fish, raw vegetables), schedule delivery on the day before or the day of use.
Minimize the number of delivery visits while satisfying the above conditions.

### [Output Format]

For each date, list the delivery time window and the ingredient names to be delivered at that time as bullet points.”

The server again invokes the generative AI model with the second prompt sentence and parses the resulting delivery schedule text. The server uses pattern matching and name resolution to map ingredient names to the product candidate table entries and constructs delivery schedule data specifying date, time window, and product quantities. The server then uses this schedule data to formulate actual order requests to external ordering and delivery services. By converting AI outputs to a canonical schedule representation and then to order requests, the server ensures that complex scheduling constraints are encoded in a machine-processable way, improving the flexibility and efficiency of order planning.

The server generates a third prompt sentence for recipe and device control. The server selects target dishes, reads the associated nutritional constraints, the current emotion vectors, and the function information of the cooking apparatus. The server forms a detailed text that describes the cooking apparatus capabilities and required output format, such as:

“For the following dish in the menu, create a recipe optimized for automatic cooking in a smart cooker.

### [Dish Name and Summary]

Dinner: Japanese-style simmered chicken and vegetables (for diabetes, reduced salt)

### [Cooking Robot Capabilities]

Heating temperature: 40-200° C. adjustable
Cooking modes: simmer, steam, stir-fry
Stirring: ON/OFF switchable, 2 speed levels
Timer: up to 120 minutes

### [User State]

The care receiver has high anxiety, so make sure the appearance and flavor are close to a typical Japanese simmered dish.
The caregiver has high fatigue, so minimize knife work and long periods of watching over the cooker.

### [Output Format]

1. List of ingredients and quantities (for two servings)
2. Step-by-step instructions to the cooking robot:
   Heating temperature (° C.)
   Heating time (minutes)
   Stirring on/off and speed
   Ingredient addition timings
3. If manual work is required, clearly describe its content and timing.”

The server transmits this prompt sentence to the generative AI model and receives a recipe text that includes explicit step-by-step instructions. The server parses the text using known headings and patterns, converting each step into structured cooking procedure data. Each step record may include fields: step index, mode, target temperature, duration, stirring status and speed, and ingredient addition action. By mandating an explicit structure through the prompt and strict parsing, the server converts qualitative natural-language instructions into deterministic procedural control sequences.

The server translates the cooking procedure data into low-level control commands for the cooking apparatus. The server maps each step's mode, temperature, and duration to device-specific commands that may be implemented as messages in a robot middleware protocol. The server also includes parameters that control operation speed and, indirectly, operation sound, such as stirrer motor speed.

The server adjusts these parameters based on emotion vector data: for example, when anxiety is high, the server chooses lower stirrer speeds and smoother temperature ramps to avoid sudden noises.

When caregiver fatigue is high, the server configures the notification system to send only essential updates, reducing cognitive load and message volume.

The cooking apparatus executes these control commands. The cooking apparatus comprises one or more temperature sensors, motor controllers, and an embedded processor. The apparatus monitors actual heating profiles and motor states and sends measurements back to the server. The server receives temperature data and operation state data and compares them against expected values defined in the cooking procedure. If the server detects a deviation such as a temperature that does not reach the expected range within a certain time, the server modifies subsequent control commands, for example by extending the heating time or increasing the temperature setpoint. This creates a closed-loop control system where the computer not only issues commands but also continuously adjusts its behavior based on sensor feedback.

The user views the cooking progress on the terminal. The terminal receives status updates from the server and displays messages such as “Cooking started,” “10 minutes remaining,” and “Please plate the dish.” The server may generate these messages at a rate governed by the emotion vector data so as to avoid overwhelming the user. The user performs only those manual actions indicated by the system, such as adding ingredients at a specific step or serving the finished dish.

After the meal, the user enters satisfaction evaluation data through the terminal. The user rates aspects such as taste, ease of eating, and overall satisfaction using numerical scales. The terminal sends these scores to the server. Optionally, the user also performs a new condition check, allowing the system to compute post-meal emotion vector data.

The server stores the satisfaction evaluation data and any post-meal emotion vectors into feedback tables. The server computes emotion changes by subtracting pre-meal emotion vectors from post-meal emotion vectors, yielding, for example, an increase or decrease in joy or anxiety. The server then performs periodic statistical analysis on accumulated feedback and emotion history. The server groups data by menu patterns or recipe features—for example by dish type, inclusion of certain ingredients, or cooking method—and computes average changes in emotion indices and satisfaction.

The server uses clustering or regression algorithms to identify patterns that correlate with positive outcomes.

The server incorporates these analysis results into the generation of subsequent prompt sentences. For example, when the server detects that menus including fish-based Japanese dishes consistently increase joy for a specific care receiver, the server adds conditions such as “prioritize fish-based Japanese dishes that were previously associated with increased joy for this patient” to future menu prompts. Similarly, if certain cooking profiles (for example longer simmering at lower temperatures) are associated with higher satisfaction, the server instructs the generative AI model to favor those patterns. In this way, the server refines the prompt content at a programmatic level, thereby steering the generative AI model toward solutions that have empirically improved emotional outcomes.

This architecture produces several technical advantages. The server reduces redundant computation by maintaining and reusing structured intermediate data such as nutritional profiles, emotion vectors, and cooking procedures, rather than repeatedly re-deriving such information from raw inputs. The server improves processing speed by executing specialized algorithms for each modality (image, audio, text) and using vectorized numerical operations, while avoiding repeated manual intervention.

The server increases accuracy and robustness by combining multimodal emotion signals with a weighted integration method, rather than relying on any single noisy source. The server reduces communication load and latency by transmitting compact structured representations between modules rather than large raw media whenever feasible, and by using the generative AI model only at well-defined interfaces.

The server's method of programmatically generating prompt sentences and parsing generative AI model outputs transforms what would otherwise be an abstract text-generation feature into a deterministic computational component integrated into the data-processing pipeline. The server does not merely automate human planning; it develops a machine-executable representation of constraints, targets, and patterns that cannot be practically maintained by human operators in real time. The closed feedback loop, wherein the server updates prompt content and control parameters based on empirical outcomes, further improves the functioning of the computer system by reducing the frequency of low-quality outputs and by converging toward parameter settings that yield higher acceptance and fewer corrections. As a result, the system advances computer technology by integrating multimodal machine learning, dynamic prompt generation, and real-time device control into a unified, adaptive architecture that achieves improved performance in terms of processing speed, accuracy, and resource utilization compared to conventional rule-based or static systems.

Multiple variations are possible within this framework. The server may use alternative neural network architectures for emotion analysis, such as attention-based vision transformers for expression analysis or convolutional-recurrent hybrids for audio classification. The server may use different generative AI backends, including locally hosted models or third-party services, so long as prompt sentences and outputs are managed according to the described structured protocol. The cooking apparatus may be replaced by other network-controllable appliances, such as ovens or multi-zone cookers, with capabilities described via function information. The weighting scheme for multimodal emotion integration may be static or dynamically learned through training on historical data. The feedback analysis component may use different statistical or machine learning techniques to infer high-yield patterns. In each case, the server, terminal, and cooking apparatus operate cooperatively to implement the claimed system by executing the defined data transformations, analysis algorithms, prompt-based generative interactions, and device control mechanisms.

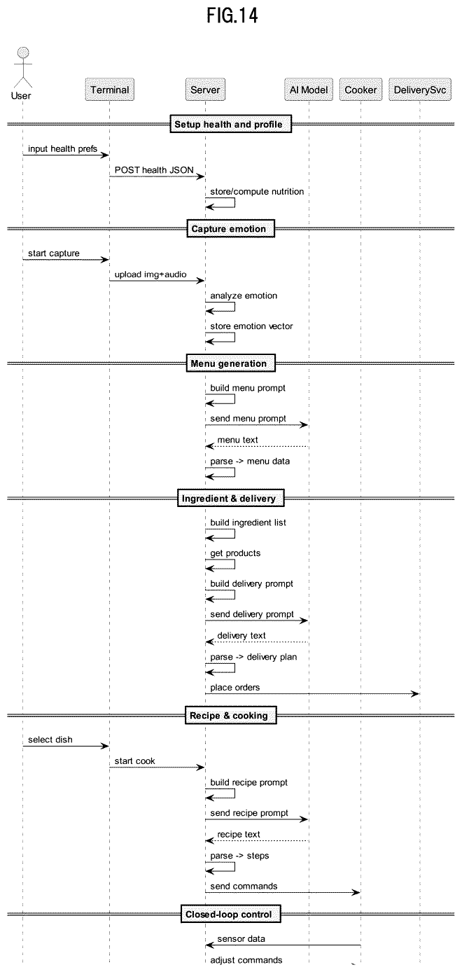

The following describes the processing flow using FIG. 14.

### Step 1

User operates the terminal to input health and preference information.

User opens a health information screen on the terminal and enters age, sex, height, weight, existing diseases, medication information, allergy information, swallowing function, and food preferences using on-screen forms.

Input: Raw user entries (form fields) on the terminal.

Terminal converts these entries into a structured JSON object with key-value pairs (for example, “age”: 82, “diseases”: [“type2_diabetes”], “allergies”: [“nuts”]), and performs basic local validation such as checking that mandatory fields are not empty.

Output: Validated JSON payload containing health and preference attributes, ready to be sent to the server.

### Step 2

Terminal transmits the health and preference JSON to the server, and the server stores normalized health data.

Input: JSON payload containing health and preference attributes received via HTTPS.

Server receives the JSON at an API endpoint, validates the structure and data types against predefined schemas, and rejects or corrects inconsistent values. Server then normalizes free-text fields using mapping tables (for example, mapping “nut allergy” and “peanut allergy” to unified codes) and splits the JSON into multiple relational records for tables such as patient, medical condition, medication, allergy, and preference. Server executes database insert or update operations using SQL commands.

Output: Normalized and stored health data records associated with a patient identifier in the database.

### Step 3

User initiates an emotion capture session, and the terminal records image and audio data.

User opens a condition-check screen on the terminal, aligns the camera to capture the care receiver's face, and taps a capture button.

Input: Live camera frames and microphone audio stream obtained by the terminal.

Terminal samples multiple image frames over a fixed duration, encodes them as compressed image files, and records audio at a predetermined sampling rate, encoding it as an audio file. Terminal packages these files along with metadata (patient identifier and timestamp) into a multipart HTTP request.

Output: Multipart request containing image files, an audio file, and metadata, transmitted to the server.

### Step 4

Server stores the uploaded media and indexes it for analysis.

Input: Multipart HTTP request carrying image files, an audio file, and metadata.

Server parses the multipart payload, writes the binary files to a media storage location, and records entries in a media index table with pointers to file paths, patient identifiers, and capture timestamps.

Server sets processing flags indicating that the media is awaiting emotion analysis.

Output: Persistent media files and corresponding media index records ready for subsequent emotion processing.

### Step 5

Server performs facial expression analysis to derive expression-based emotion scores.

Input: Paths to stored face image files referenced by the media index.

Server loads each image, applies a face detection model to locate facial regions, and crops and resizes these regions to the required input size for a convolutional expression classifier. Server then normalizes pixel values and feeds each processed face image into the classifier, which outputs probability distributions over expression categories like joy, anger, sadness, surprise, and neutral.

Server aggregates the per-image probability vectors for each session via averaging or weighted averaging to reduce noise.

Output: Expression-based emotion scores, represented as numeric values (for example, probabilities or 0-1 scores) for each emotion category for the session.

### Step 6

Server performs audio emotion analysis and speech recognition.

Input: Path to the stored audio file, and associated metadata.

Server loads the audio waveform, applies pre-processing such as normalization and silence trimming, and computes time-frequency features like mel-spectrograms. Server inputs these features into an audio emotion classifier, which outputs numeric scores for emotions such as joy, anxiety, anger, and calm. Server then passes the same audio waveform to a speech recognition engine, which converts the speech into a text transcript.

Output: Audio-based emotion scores and a recognized text transcript of the recorded speech.

### Step 7

Server performs text emotion analysis on the recognized speech.

Input: Recognized text transcript obtained from speech recognition.

Server tokenizes the transcript, segments it as required, and feeds it into a text-based emotion classification model. The model processes token embeddings through multiple layers and outputs sentiment and emotion scores, including positive versus negative polarity, and specific emotions or states such as anxiety or stress. Server optionally maps these model outputs to a normalized scale, such as 0-10.

Output: Text-based emotion scores for sentiment and emotion categories derived from the transcript.

### Step 8

Server integrates multimodal emotion scores into a unified emotion vector.

Input: Expression-based emotion scores, audio-based emotion scores, and text-based emotion scores for the same session.

Server applies predefined or dynamically learned weights to each modality, taking into account factors such as signal quality or completeness. Server computes weighted sums for each target emotion dimension (for example, joy, anxiety, stress, fatigue, and motivation) and then linearly rescales them into a standardized range like 0-10. Server constructs an emotion vector by placing each rescaled value in a fixed position within a vector structure and associates it with patient and caregiver identifiers. Server stores the emotion vector in an emotional state table with a timestamp.

Output: Emotion vector data capturing the current emotional state as multi-dimensional numeric values stored in the database.

### Step 9

Server calculates nutritional profile data from health records.

Input: Health data records (patient, medical condition, medication, allergy, and preference records) retrieved from the database for a specified patient.

Server loads nutritional standard tables into memory, and uses health attributes such as age, sex, height, weight, and activity level to compute basal metabolic rate and total energy requirement.

Server additionally applies disease-specific rules, such as modifying macronutrient ratios or limiting specific nutrients, and builds a prohibited ingredient list from allergy and medical condition information. Server compiles target energy, macronutrient ratios, micronutrient targets, and constraints into a structured profile object.

Output: Nutritional profile data stored in a nutrition profile table, linked to the patient identifier.

### Step 10

Server generates a first prompt sentence for menu generation using nutritional and emotion data.

Input: Nutritional profile data and the latest emotion vectors for the care receiver and caregiver.

Server selects template text for menu generation and populates placeholders with concrete values, including target energy, macronutrient ranges, prohibited ingredients, and numeric indicators of anxiety, motivation, or fatigue. Server configures a clear output format section in the prompt, specifying the required structure of the generated menu. The result is a coherent, human-readable prompt sentence that precisely encodes health and emotion constraints.

Output: First prompt sentence text ready to be submitted to a generative AI model.

### Step 11

Server calls the generative AI model with the first prompt sentence and constructs structured menu data.

Input: First prompt sentence text.

Server transmits the prompt to a generative AI endpoint, specifying generation parameters such as maximum tokens and sampling strategy. Server receives the returned menu proposal text, which describes proposed dishes for each meal category, their ingredients, and approximate nutrient information. Server parses this text by detecting labeled sections (for example, “Breakfast”, “Lunch”, “Dinner”) and bullet lists, extracting dish names, descriptions, ingredient names, and approximate calories or carbohydrate amounts. Server converts these extracted items into structured records in menu and dish tables.

Output: Structured menu data, including meals and associated dishes with ingredient lists and nutritional estimates.

### Step 12

Server computes an aggregated ingredient list from menu data.

Input: Structured menu and dish data for a defined time horizon (for example one day or one week).

Server retrieves each dish's base ingredient requirements from a recipe or ingredient mapping table and multiplies per-serving quantities by the number of servings required. Server groups ingredient entries by ingredient identifier and sums the total amounts. Server then consults ingredient master data to convert these total amounts into purchase units (for example packs or pieces), rounding up to the nearest full unit.

Output: Ingredient list data specifying ingredient identifiers, required amounts, and purchase units.

### Step 13

Server obtains product candidates and prepares information for delivery planning.

Input: Ingredient list data containing required ingredients and quantities.

Server sends queries to external product information services for each ingredient, specifying names or codes and optional filters. Server receives product lists with stock status, price, processing form (fresh, pre-cut, frozen, pre-cooked), and delivery options. Server filters out unavailable or unsuitable items and compiles candidate product records linked to ingredients. Server also calculates preliminary mappings between ingredients, candidate products, and planned usage dates from the menu schedule.

Output: Product candidate data associated with each ingredient and a schedule of ingredient usage by date.

### Step 14

Server generates a second prompt sentence for delivery schedule planning.

Input: Ingredient usage schedule, product candidate data, caregiver emotion vector, and user-defined delivery preferences.

Server aggregates ingredient requirements by day, associates each ingredient with candidate products and processing forms, and reads the caregiver's fatigue and stress values. Server also loads the user's preferred delivery time windows. Server constructs a text prompt describing ingredients needed on each date, the caregiver's state, freshness and burden-reduction constraints, and the desired output format (for example, a list of dates and time windows with grouped ingredients).

Output: Second prompt sentence text designed to elicit a delivery schedule proposal from the generative AI model.

### Step 15

Server calls the generative AI model with the second prompt sentence and builds delivery schedule data.

Input: Second prompt sentence text.

Server transmits the prompt to the generative AI model and receives a delivery schedule proposal text. Server parses this text by identifying date headings, time window descriptions, and lists of ingredients per delivery slot. Server matches ingredient names to stored ingredient and product identifiers using normalization and, when necessary, fuzzy matching. Server then creates structured delivery schedule records linking dates, time windows, and specific product orders with quantities.

Output: Delivery schedule data stored in a delivery schedule table, suitable for generating external order requests.

### Step 16

Server issues order requests to external order and delivery services based on delivery schedule data.

Input: Delivery schedule data and associated product candidate records.

Server iterates through delivery events, constructs order payloads that include product identifiers, quantities, delivery date and time window, and delivery address, and sends these payloads to external ordering systems via their APIs. Server receives responses containing order identifiers and status codes, and updates the delivery schedule and order tracking tables with this information.

Output: Confirmed order records and updated delivery schedule entries linked to external order identifiers.

### Step 17

Server generates a third prompt sentence for recipe generation and device control.

Input: Selected dish data from the menu, nutritional profile data, current emotion vectors, and function information of the cooking apparatus.

Server loads the cooking apparatus capabilities, such as supported modes, temperature range, stirring options, and maximum timer duration. Server reads the care receiver's anxiety and the caregiver's fatigue and incorporates these into instructions about acceptable complexity and manual work. Server populates a text template that demands detailed, stepwise cooking instructions and clearly specified parameters for each step.

Output: Third prompt sentence text to request a device-optimized recipe from the generative AI model.

### Step 18

Server calls the generative AI model with the third prompt sentence and parses the recipe into cooking procedure data.

Input: Third prompt sentence text.

Server sends the prompt to the generative AI endpoint and receives a recipe text that includes an ingredient list and sequential instructions. Server parses the text by locating the ingredient section and the list of steps. For each step, server extracts actions, cooking mode, heating temperature, duration, stirring on or off, stirring speed, and ingredient addition timings. Server encodes each step as a structured record in a cooking procedure table with fields for step index, operation type, parameters, and manual action flags.

Output: Cooking procedure data representing a sequence of parameterized cooking steps.

### Step 19

Server converts cooking procedure data and emotion vectors into device control commands.

Input: Cooking procedure data for a specific dish and current emotion vectors for the care receiver and caregiver.

Server maps each procedure step to one or more low-level control commands understood by the cooking apparatus, such as commands to set a mode, adjust temperature, start or stop a timer, and toggle stirring at a specified speed. Server adjusts parameters such as motor speed and temperature ramp rates based on emotion values; for instance, server chooses slower stirring speeds and gentler heating when anxiety is high to reduce operation sound. Server also determines notification patterns for user messages based on caregiver fatigue.

Output: A sequence of device-specific control commands and notification schedule parameters ready to be sent to the cooking apparatus and terminal.

### Step 20

Server executes closed-loop control of the cooking apparatus.

Input: Sequence of control commands, device capability information, and real-time sensor data from the cooking apparatus.

Server transmits control commands to the cooking apparatus through a communication interface and tracks execution state (for example current step index). As the cooking apparatus reports temperature measurements and operation states, server compares these values to expected ranges defined in the cooking procedure. If discrepancies occur, such as insufficient temperature, server computes corrective actions like extending heating time or adjusting target temperature and sends updated commands. Server simultaneously generates status messages for the terminal, formatted according to the notification parameters derived from emotion vectors.

Output: Adjusted command stream driving the cooking apparatus through the recipe steps and status updates delivered to the terminal for user awareness.

### Step 21

User reviews cooking progress and performs indicated manual actions via the terminal.

User observes status messages on the terminal's display, such as “Add vegetables now” or “Prepare plates,” and follows those instructions at the indicated times.

Input: Status messages and prompts received by the terminal from the server.

Terminal displays these prompts and optionally plays audio guidance. User then executes manual tasks like adding ingredients or plating the dish at the appropriate moments, based on the instructions.

Output: Correctly timed manual interventions that complement automated device control to complete the cooking process.

### Step 22

User provides post-meal feedback, and the server computes emotion changes and updates models.

User enters satisfaction ratings for parameters such as taste, ease of eating, and overall satisfaction on the terminal after the meal, and may initiate another emotion capture session.

Input: Satisfaction evaluation data and, optionally, new image and audio recordings from the terminal.

Terminal transmits the ratings as structured data and, if captured, new media to the server. Server stores satisfaction scores in a feedback table and processes any new media through the emotion analysis pipeline to obtain post-meal emotion vectors. Server compares pre-meal and post-meal emotion vectors to compute emotion changes, such as the change in joy or anxiety indices, and logs these changes along with the corresponding menu and recipe identifiers. Server periodically analyzes accumulated logs using statistical or machine learning methods to identify menu and cooking patterns that tend to produce favorable emotion changes. Server then updates internal prompt-generation parameters and rule weights so that future prompt sentences for the generative AI model incorporate these learned preferences and constraints.

Output: Updated feedback records, emotion change metrics, and refined parameters that influence subsequent nutritional planning, prompt sentences, and device control behavior.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user.

Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

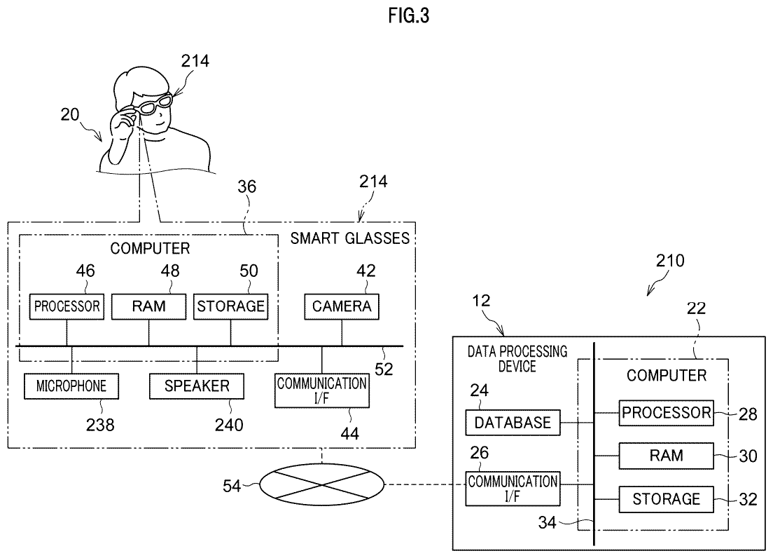

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein.

The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

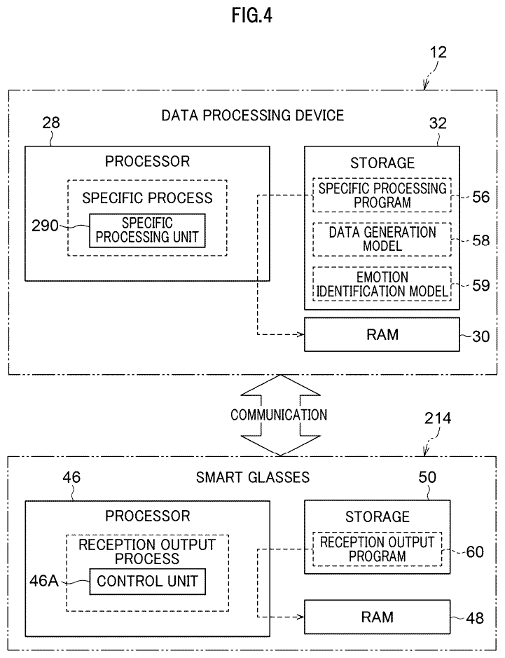

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

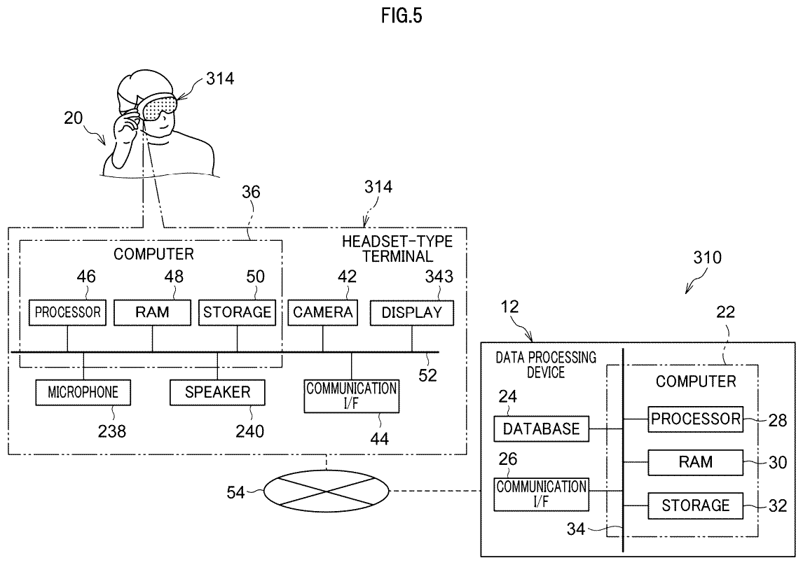

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

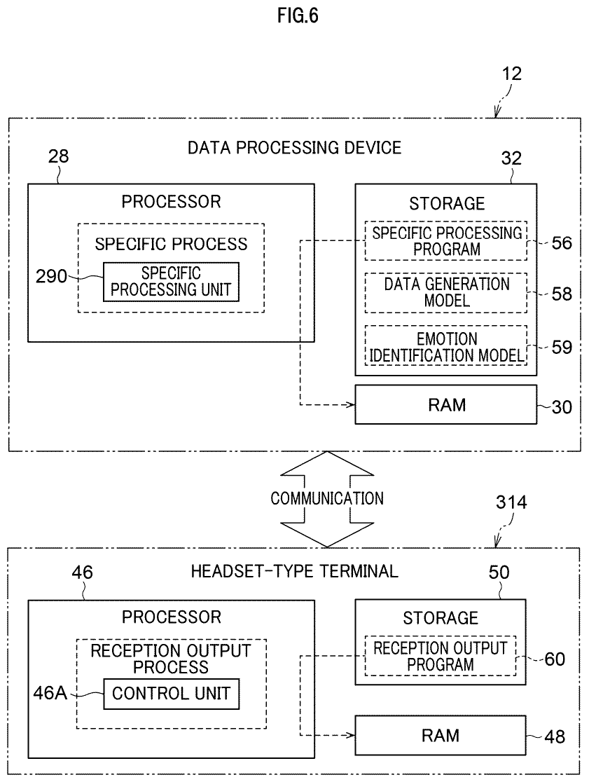

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

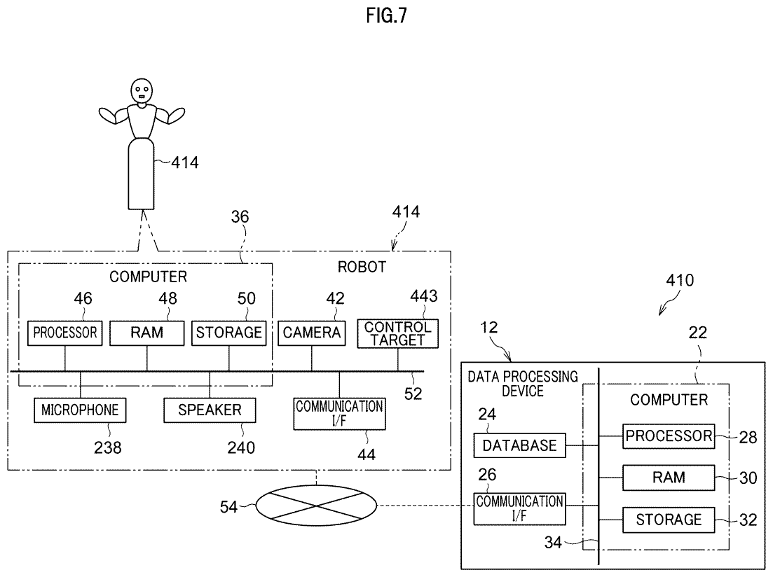

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

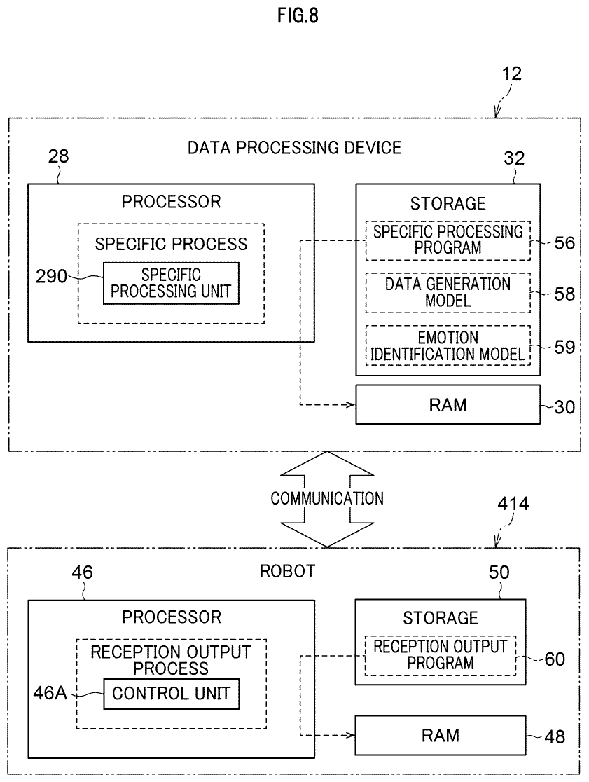

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

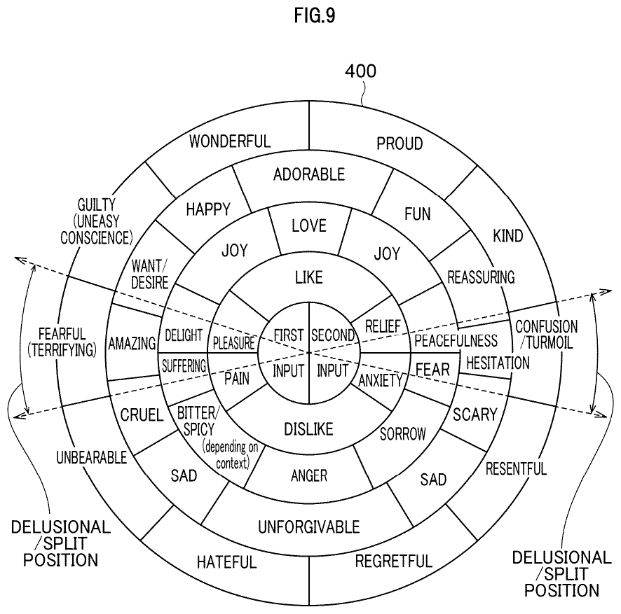

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

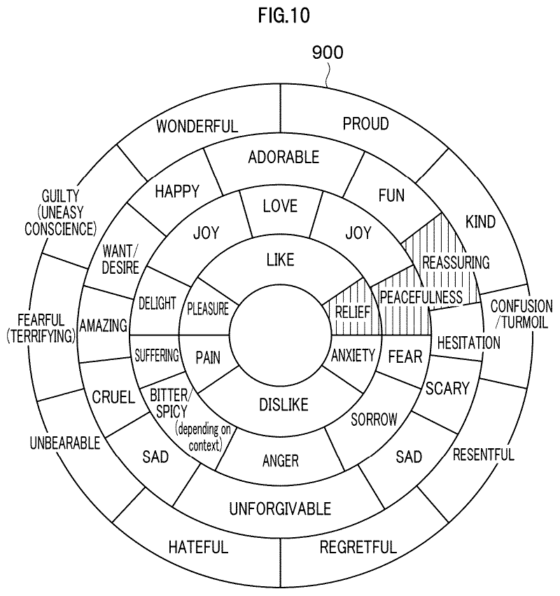

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to acquire, via an input interface, household composition information and health-related information for
each member of a household, and store the household composition information and the health-related information in a storage device,
perform numerical computation based on the household composition information, the health-related information, and nutrition reference information stored in the storage device, to calculate nutrition requirement information for each member,
generate structured information including the nutrition requirement information, the household composition information, and the health-related information, convert at least part of the structured information into a character string as a prompt sentence, transmit the prompt sentence to a generative AI model, receive response data from the generative AI model, and generate menu information for a predetermined period based on the response data,
extract ingredient information for a plurality of dishes included in the menu information, perform aggregation processing and unit conversion processing for each ingredient to calculate a required ingredient amount, acquire ingredient candidate information corresponding to the required ingredient amount from an external product information providing device, and generate ingredient list information,
acquire, based on the ingredient list information and delivery-preference time-band information of a user stored in the storage device, delivery-slot information from an external goods-delivery management device, automatically determine a delivery schedule consistent with the delivery-preference time-band information, and transmit order information to the goods-delivery management device,
generate cooking procedure data indicating cooking steps for each dish based on the menu information and the nutrition requirement information, and generate structured data including operation parameters for a cooking apparatus from the cooking procedure data,
generate a sequence of control commands including temperature, time, stirring speed, and heating state based on the structured data, and transmit the sequence of control commands to the cooking apparatus,
receive temperature information, time information, and operation-state information from the cooking apparatus, compare a target value in the sequence of control commands with the temperature information, the time information, and the operation-state information to determine a cooking state,
and generate, according to a determination result, additional control commands including extension of cooking time or change of temperature and transmit the additional control commands to the cooking apparatus, and
store, in association, at least one of the prompt sentence and the response data of the generative AI model, and the household composition information and the health-related information, in the storage device as learning data, and update a generation condition of a subsequent prompt sentence based on the learning data.

### Supplementary 2

The system according to supplementary 1,
wherein the processor is configured to
compare ingredient information included in menu-candidate information, which is received from the generative AI model by the processor in response to the prompt sentence, with allergy information and intake-restriction information included in the health-related information, detect a dish candidate including a prohibited ingredient, exclude the dish candidate from the menu-candidate information,
generate an additional prompt sentence requesting generation of an alternative dish that does not include the prohibited ingredient, transmit the additional prompt sentence to the generative AI model,
receive response data corresponding to the additional prompt sentence, and regenerate the menu information using the response data.

### Supplementary 3

The system according to supplementary 1,
wherein the processor is configured to
acquire device-capability information including model information and function information of the cooking apparatus, generate a prompt sentence including the device-capability information and the menu information, transmit the prompt sentence to the generative AI model, receive recipe structured data including a plurality of cooking steps with time, temperature, stirring conditions, and user-intervention instructions from the generative AI model, generate the sequence of control commands based on the recipe structured data, and, during execution of the sequence of control commands, sequentially change the sequence of control commands according to sensor information received from the cooking apparatus.

### Application Example 1

### Supplementary 1

A system comprising a processor,
wherein the processor is configured to
acquire health information of a care recipient via a health information acquisition unit that provides an input interface for age, sex, health condition, allergy information, dietary preference, and special nutritional need,
calculate, by a nutrition information calculation unit, a nutritional requirement for the care recipient on the basis of the acquired health information by using a predetermined nutrition algorithm,
generate, by a menu information generation unit, meal plan information for the care recipient on the basis of the calculated nutritional requirement and the dietary preference,
generate, by an ingredient information generation unit, ingredient procurement information by aggregating ingredient information included in the generated meal plan information for each dish,
coordinate, by a procurement cooperation unit, ordering information and delivery schedule information of food ingredients with an external procurement service on the basis of the ingredient procurement information so as to automatically perform ordering and delivery time adjustment of the food ingredients,
generate, by a cooking procedure generation unit, cooking procedure information on the basis of the meal plan information and the calculated nutritional requirement, and convert the cooking procedure information into control procedure information that conforms to an operating specification of a cooking apparatus,
generate, by a cooking instruction generation unit, a start instruction, an operation instruction, and a parameter change instruction for the cooking apparatus on the basis of the control procedure information,
control, by a cooking apparatus control unit, a heating mechanism, a stirring mechanism, and a sensor of the cooking apparatus in real time in accordance with the generated instructions, and perform feedback control of temperature, time, and operation state on the basis of acquired cooking state information,
cooperate, by a generative artificial intelligence cooperation unit, with a generative artificial intelligence model in at least one of the menu information generation unit, the ingredient information generation unit, and the cooking procedure generation unit, the generative artificial intelligence cooperation unit being configured to generate a prompt sentence expressed in natural language by using structured data including the health information, the nutritional requirement, the meal plan information, and ingredient information as input conditions, to input the prompt sentence into the generative artificial intelligence model, to obtain candidate menu information, candidate ingredient information, or candidate cooking procedure information from the generative artificial intelligence model, and to verify and correct the obtained candidate information on the basis of an allergen constraint, a disease constraint, and a nutrition constraint, and
control, by a prompt control unit, the prompt sentence to be input into the generative artificial intelligence model such that the prompt sentence includes at least one of a health state of the care recipient, a restricted ingredient, a required nutrient, period information, and the operating specification of the cooking apparatus, and convert structured output obtained from the generative artificial intelligence model into an internal data format used in at least one of the menu information generation unit, the ingredient information generation unit, and the cooking procedure generation unit.

### Supplementary 2

The system according to supplementary 1,
wherein the processor is configured to, in the generative artificial intelligence cooperation unit, generate a prompt sentence expressed in natural language on the basis of the health information of the care recipient, the nutritional requirement calculated by the nutrition information calculation unit, and the meal plan information generated by the menu information generation unit, the prompt sentence being configured to request integration of a list of dishes and a list of food ingredients required for the dishes over an entire meal plan period, to input the prompt sentence into the generative artificial intelligence model, to obtain structured data including a candidate ingredient list grouped by category and an explanatory sentence from the generative artificial intelligence model, to cause the ingredient information generation unit to automatically generate the ingredient procurement information on the basis of the obtained structured data, and to cause the procurement cooperation unit to automatically perform ordering and delivery schedule adjustment with the external procurement service by using the ingredient procurement information.

### Supplementary 3

The system according to supplementary 1,
wherein the processor is configured to cause the cooking procedure generation unit to generate a prompt sentence expressed in natural language on the basis of dish information included in the meal plan information generated by the menu information generation unit and the operating specification of the cooking apparatus, the prompt sentence including at least a target temperature, cooking time, stirring speed, and ingredient addition order for each cooking step, to cause the generative artificial intelligence cooperation unit to input the prompt sentence into the generative artificial intelligence model and to obtain machine-readable cooking procedure information including a step number, a step description, a temperature value, a time value, and safety notes from the generative artificial intelligence model, to cause the cooking apparatus control unit to perform feedback control by comparing the obtained cooking procedure information with temperature information and operation state information acquired from the cooking apparatus, and, when an abnormal cooking state is detected during cooking, to cause the cooking apparatus control unit to automatically output at least one of a stop instruction for the cooking apparatus and a cooking condition change instruction, and to cause the generative artificial intelligence cooperation unit to generate, on the basis of the obtained cooking procedure information, an explanation message expressed in natural language for a user.

### Example 2

### Supplementary 1

A system comprising a processor,
wherein the processor is configured to acquire health state data for each member of a household as input health information, and store the health state data in an information storage device as structured records, and refer to nutrition science information stored in the information storage device and calculate required nutrient amounts for each household member based on the health state data by using a nutrition balance calculation function, and
input at least one of audio data, image data, and text data, acquired via a terminal device, into a machine learning model or a generative AI model, estimate an emotional state of a user, and generate emotional parameters including at least a stress level and a fatigue level by using an emotion estimation function, and
generate, by using a menu generation function, a prompt sentence including family composition information, health state information, nutrition condition information, preference information, and the emotional parameters, based on the required nutrient amounts calculated by the nutrition balance calculation function, the emotional parameters, and at least one of season information and event information, input the prompt sentence into a generative AI model to generate menu candidates for the household, verify whether nutrition values of the generated menu candidates fall within target ranges, and, when a deviation is detected, regenerate a corrected prompt sentence including modified constraints and re-input the corrected prompt sentence into the generative AI model so as to dynamically optimize the menu candidates in accordance with both nutrition balance and the emotional state, and
extract required ingredients based on dishes included in the optimized menu candidates, refer to ingredient information stored in the information storage device and to stock information and price information acquired from an external product provision device, and, by using an ingredient list generation function, select, from among a plurality of ingredient forms including at least fresh ingredients and partially prepared ingredients, an optimal ingredient form and a purchase source while applying a higher weight to ingredient forms that reduce a cooking burden when at least one of the emotional parameters indicates a high burden, thereby generating an ingredient list, and determine, by using an ordering and delivery cooperation function, at least one delivery date and time based on the ingredient list and user schedule information stored in the information storage device, and transmit order information including the ingredient list and the at least one delivery date and time to an external ordering and delivery service device, and
generate, by using a recipe generation function, a recipe-generation prompt sentence based on a menu selected from the optimized menu candidates, cooking apparatus specification information stored in the information storage device, and the emotional parameters, the recipe-generation prompt sentence including constraints on user workload, including explicit numerical values of heating temperature and heating time for each cooking step, and distinguishing between user operations and automatic operations of a cooking apparatus, input the recipe-generation prompt sentence into a generative AI model, and generate a cooking recipe, and
extract, by using a cooking instruction function, a cooking step number, an ingredient input order, a heating temperature, a heating time, and stirring conditions from the cooking recipe, and convert the extracted information into a control command sequence interpretable by the cooking apparatus, and transmit, by using a cooking robot control function, the control command sequence to the cooking apparatus via a communication network, acquire temperature sensor information, timer information, and image information from the cooking apparatus, and, based on the acquired information, generate additional control commands including at least an extension of the heating time or a change of the temperature setting and transmit the additional control commands in real time to control automatic cooking by the cooking apparatus, and
transmit, by using a terminal cooperation function, the menu candidates, the ingredient list, order status information, and cooking recipe information to the terminal device, receive a cooking start instruction and satisfaction evaluation information from the terminal device, and store the satisfaction evaluation information in association with the emotional parameters and the menu candidates as learning data in the information storage device.

### Supplementary 2

The system according to supplementary 1,
wherein the processor is configured to
execute a learning process based on the health state data and preference data for each household member acquired via a health state data input interface, the emotional parameters estimated by the emotion estimation function, and the satisfaction evaluation information acquired via the terminal cooperation function, and update, according to the learning process, at least one of a calculation logic of the nutrition balance calculation function for the required nutrient amounts, a template and constraint conditions of the prompt sentence in the menu generation function, and weighting parameters used by the ingredient list generation function for selection between fresh ingredients and partially prepared ingredients, thereby automatically adjusting, over time, contents of prompt sentences input to each generative AI model and selection results of ingredient forms.

### Supplementary 3

The system according to supplementary 1,
wherein the processor is configured to
cause the ordering and delivery cooperation function to set a plurality of delivery dates and times by dividing, based on the ingredient list, the user schedule information, and the emotional parameters, ingredients for a simple same-day menu and ingredients for a later-day regular menu into different time slots, cause the recipe generation function to explicitly describe, in the recipe-generation prompt sentence, an upper limit on a number of user operation steps and an upper limit on a total cooking time in accordance with the cooking apparatus specification information and the emotional parameters, and cause the cooking robot control function to estimate, based on operating state information acquired from the cooking apparatus, a progress degree of each cooking step, compare an estimated cooking completion time with the user schedule information, and generate control commands for at least one of extending a warming period and shortening a part of the cooking steps when an adjustment is required.

### Application Example 2

### Supplementary 1

A system comprising a processor,
wherein the processor is configured to
receive, via an information input interface, health information and preference information regarding a care receiver, and to store the health information and the preference information in a storage device as health data associated with an identifier of the care receiver,
calculate, based on the health data, a nutritional requirement amount and a nutritional constraint condition for the care receiver, and generate nutritional profile data including at least a target energy amount, target macronutrient ratios, micronutrient targets, and a prohibited ingredient list, analyze image information, audio information, and text information related to the care receiver or a caregiver, perform expression analysis, audio emotion analysis, and text emotion analysis on the image information, the audio information, and the text information, and generate emotion vector data in which multiple emotion indices including joy, anxiety, stress, fatigue, and motivation are represented as numerical values,
generate a first prompt sentence for a generative AI model, the first prompt sentence including conditions based on the nutritional profile data and the emotion vector data, the conditions including nutritional constraints, prohibited ingredients, an emotion state of the care receiver, and an emotion state of the caregiver, transmit the first prompt sentence to the generative AI model, receive, from the generative AI model, a menu proposal text, and analyze the menu proposal text to extract, for each meal category, at least a dish name, an ingredient list, and nutritional information, and to generate menu data,
aggregate, based on the menu data, types and amounts of ingredients required for the menu data, and generate ingredient list data representing required purchase units of ingredients,
generate a second prompt sentence for the generative AI model based on the ingredient list data, the emotion vector data of the caregiver, and planned use dates of the ingredients, the second prompt sentence including at least conditions relating to delivery date and time, the number of deliveries, and processing form of the ingredients, transmit the second prompt sentence to the generative AI model, receive, from the generative AI model, a delivery schedule proposal text, analyze the delivery schedule proposal text to extract dates, time windows, and groups of ingredients, and generate delivery schedule data, and transmit, based on the delivery schedule data, order requests for the ingredients to an external order and delivery service,
generate a third prompt sentence for the generative AI model based on the menu data, the nutritional profile data, the emotion vector data, and function information of a cooking apparatus, the third prompt sentence including at least cooking conditions comprising a cooking mode, a heating temperature, a heating time, a stirring condition, and a required manual operation, transmit the third prompt sentence to the generative AI model, receive, from the generative AI model, a recipe text, and analyze the recipe text to extract a step order, a temperature, a time, a stirring condition, and ingredient addition timing, and to generate cooking procedure data,
generate, based on the cooking procedure data and the emotion vector data, a sequence of control commands including at least a cooking mode setting command, a temperature setting command, a timer setting command, and a stirring control command for the cooking apparatus, adjust at least one control parameter relating to operation speed, operation sound, or notification frequency of the cooking apparatus according to the emotion state of the care receiver and the emotion state of the caregiver, add the adjusted control parameter to the sequence of control commands, and transmit the sequence of control commands to the cooking apparatus so as to cause the cooking apparatus to operate in an automatic or semi-automatic manner,
monitor temperature data and operation state data received from the cooking apparatus, compare the temperature data and the operation state data with predetermined threshold values, and, based on a comparison result, modify the sequence of control commands dynamically to perform at least extension of a heating time or change of a heating temperature, and
obtain, after provision of a meal, satisfaction evaluation data and post-meal emotion vector data, analyze a relationship between menu patterns and emotion changes based on the satisfaction evaluation data and the post-meal emotion vector data, and reflect an analysis result as a condition in generation of at least one of the first prompt sentence, the second prompt sentence, and the third prompt sentence, so that subsequent prompt sentences for the generative AI model are adjusted in accordance with long-term preference tendencies and emotion tendencies of the care receiver.

### Supplementary 2

The system according to supplementary 1,

wherein the processor is configured to
perform, in the analyzing of the image information, the audio information, and the text information, a face detection process and a facial expression classification process on the image information to calculate an expression-based emotion score, perform an acoustic feature extraction process and an audio emotion classification process on the audio information to calculate an audio-based emotion score, perform an automatic speech recognition process and a text emotion classification process on the text information to calculate a text-based emotion score, apply weighting to the expression-based emotion score, the audio-based emotion score, and the text-based emotion score, and integrate the scores to generate the emotion vector data.

### Supplementary 3

The system according to supplementary 1,
wherein the processor is configured to
adjust, for each of the first prompt sentence, the second prompt sentence, and the third prompt sentence, content of the prompt sentence dynamically based on the nutritional profile data, the emotion vector data, and the analysis result of the relationship between menu patterns and emotion changes, and to add, to the prompt sentence, a condition corresponding to at least one of past menu histories and past emotion change histories for the care receiver, such that an output of the generative AI model is adapted to long-term preference tendencies and emotion tendencies of the care receiver.

What is claimed is:

1. A system comprising:
circuitry configured to:
receive, via a communication interface coupled to a packet-switched network, attribute data records associated with a plurality of user profiles, each attribute data record including a plurality of user-specific parameters;
compute, based on the attribute data records and reference data stored in a memory, per-user requirement values for each of the plurality of user profiles;
generate, by inputting the per-user requirement values into a data generation model, a composite output plan satisfying constraint conditions derived from the per-user requirement values;
generate, based on the composite output plan, instruction data including a sequence of operational parameters; and
transmit, via the communication interface, the instruction data to a controlled apparatus over the packet-switched network to cause the controlled apparatus to execute operations in accordance with the operational parameters.

2. The system according to claim 1, wherein the circuitry is further configured to generate the composite output plan by generating a prompt character string based on the per-user requirement values, inputting the prompt character string into the data generation model, and parsing a structured output received from the data generation model.

3. The system according to claim 2, wherein the data generation model comprises a large language model, and the structured output comprises a data object including a plurality of entries each associated with a respective one of the plurality of user profiles.

4. The system according to claim 3, wherein the circuitry is further configured to validate the composite output plan by comparing each entry of the data object against the constraint conditions, and upon detecting a constraint violation, generate a revised prompt character string including the constraint violation and re-input the revised prompt character string into the large language model to obtain a revised composite output plan.

5. The system according to claim 4, wherein the attribute data records comprise physiological condition data, allergen information, and dietary preference data for each of the plurality of user profiles, and the constraint conditions include allergen exclusion criteria and caloric intake thresholds derived from the physiological condition data.

6. The system according to claim 5, wherein the composite output plan comprises a nutritionally balanced meal plan, and the circuitry is further configured to derive, from the meal plan, an aggregated ingredient list by consolidating ingredients across entries of the meal plan and converting ingredient quantities to standardized units.

7. The system according to claim 1, wherein the circuitry is further configured to receive, via the communication interface, sensor data from the controlled apparatus during execution of the operations, and adjust the operational parameters of the instruction data based on a comparison between the sensor data and target values associated with the composite output plan.

8. The system according to claim 7, wherein adjusting the operational parameters comprises computing a difference between the sensor data and the target values, and generating a corrective command based on the difference to modify at least one of a rate, a duration, or an intensity of the operations executed by the controlled apparatus.

9. The system according to claim 8, wherein the circuitry is further configured to detect an abnormal state when the difference exceeds a threshold, and upon detecting the abnormal state, transmit a stop command or a modified command sequence to the controlled apparatus via the communication interface.

10. The system according to claim 9, wherein the controlled apparatus comprises a cooking apparatus having a heating mechanism, a stirring mechanism, and temperature sensors, and the operational parameters include temperature settings, stirring speed values, and duration values for each step of the sequence of operational parameters.

11. The system according to claim 1, wherein the circuitry is further configured to receive, via the communication interface, multimodal sensor data from a client terminal associated with one of the plurality of user profiles, and estimate an emotion value by applying an emotion identification model to the multimodal sensor data.

12. The system according to claim 11, wherein the multimodal sensor data comprises image data and audio data, and the emotion identification model comprises a convolutional neural network configured to process the image data and a recurrent neural network configured to process the audio data, and the emotion value comprises a vector of weighted scores across a plurality of emotion categories.

13. The system according to claim 12, wherein the circuitry is further configured to adjust a parameter of the prompt character string input to the data generation model based on the emotion value, the parameter comprising at least one of a complexity constraint, a time constraint, or a preference weighting.

14. The system according to claim 13, wherein the circuitry is further configured to estimate a post-operation emotion value after the controlled apparatus completes the operations, compute a satisfaction score based on a difference between the emotion value and the post-operation emotion value, and update a prompt template stored in the memory based on the satisfaction score.

15. The system according to claim 1, wherein the circuitry is further configured to derive, from the composite output plan, an aggregated item list identifying items and corresponding quantities, and transmit the aggregated item list to an external service system over the packet-switched network to initiate automated procurement of the identified items.

16. The system according to claim 15, wherein the circuitry is further configured to generate a delivery schedule specifying a plurality of delivery time slots based on timing constraints derived from the composite output plan, and transmit the delivery schedule to the external service system.

17. The system according to claim 16, wherein the external service system comprises an online ordering and delivery service, and the circuitry is further configured to adjust the delivery time slots based on an emotion value estimated from sensor data of a user associated with one of the plurality of user profiles.

18. A system comprising:
circuitry configured to:
receive, via a communication interface coupled to a packet-switched network, attribute data records each including age, physiological condition indicators, allergen flags, and dietary preference parameters for each member of a family;
compute per-user nutritional requirement values by applying a constraint optimization to the attribute data records and reference nutritional data stored in a memory;
generate, by inputting the per-user nutritional requirement values into a large language model as a structured prompt character string, a nutritionally balanced meal plan satisfying the per-user nutritional requirement values;
validate the meal plan by comparing each entry against allergen exclusion rules derived from the allergen flags, and upon detecting a violation, regenerate the meal plan by inputting a revised prompt character string into the large language model;
derive from the validated meal plan an aggregated ingredient list with quantities and transmit the aggregated ingredient list to an external ordering service over the packet-switched network;
generate, by inputting the meal plan and apparatus capability parameters into the large language model, procedure data indicating a sequence of cooking steps with temperature, duration, and stirring parameters; and
transmit, via the communication interface, control command data derived from the procedure data to a cooking apparatus over the packet-switched network to cause the cooking apparatus to execute the sequence of cooking steps.

19. The system according to claim 18, wherein the circuitry is further configured to receive, via the communication interface, sensor data including temperature readings and load measurements from the cooking apparatus during execution of the cooking steps, compare the sensor data against target values specified in the procedure data, and transmit corrective commands to the cooking apparatus to adjust at least one of a heating intensity, a stirring speed, or a cooking duration based on a deviation between the sensor data and the target values.

20. A method performed by circuitry of a system, the method comprising:
receiving, via a communication interface coupled to a packet-switched network, attribute data records associated with a plurality of user profiles, each attribute data record including a plurality of user-specific parameters;
computing, based on the attribute data records and reference data stored in a memory, per-user requirement values for each of the plurality of user profiles;
generating, by inputting the per-user requirement values into a data generation model, a composite output plan satisfying constraint conditions derived from the per-user requirement values;
generating, based on the composite output plan, instruction data including a sequence of operational parameters; and
transmitting, via the communication interface, the instruction data to a controlled apparatus over the packet-switched network to cause the controlled apparatus to execute operations in accordance with the operational parameters.
